(19) United States
(12) Patent Application Publication
(10) Pub. No.: US 2023/0085173 A1
(43) Pub. Date: Mar. 16, 2023

(54) PURIFICATION OF SACCHARIDES

(71) Applicant: Pfizer Inc., New York, NY (US)

(72) Inventors: Ling Chu, Suffern, NY (US); Wei Chen, Cliffside Park, NJ (US); Nishith Merchant, Parlin, NJ (US); Justin Keith Moran, West Nyack, NY (US)

(21) Appl. No.: 17/799,992

(22) PCT Filed: Feb. 17, 2021
(86) PCT No.: PCT/IB2021/051330

§ 371 Date: Aug. 16, 2022

Related U.S. Application Data
(60) Provisional application No. 63/143,795, filed on Jan. 30, 2021.
(60) Provisional application No. 63/068,338, filed on Aug. 20, 2020.
(60) Provisional application No. 62/980,134, filed on Feb. 21, 2020.

(51) Int. Cl. C08B 37/00; B01D 21/01; B01D 61/14

(52) U.S. Cl. CPC C08B 37/0003; B01D 21/01; B01D 61/146

(57) ABSTRACT

The present invention relates to methods for purifying bacterial polysaccharides, in particular for removing impurities from cellular lysates of bacteria producing polysaccharides, comprising: a) acid hydrolysis; b) a first ultrafiltration/diafiltration-(UFDF-1); b) carbon filtration; c) chromatography; and d) a second ultrafiltration/diafiltration-(UFDF-2).

### REFERENCE TO SEQUENCE LISTING

This application is being filed electronically via EFS-Web and includes an electronically submitted sequence listing in .txt format. The .txt file contains a sequence listing entitled “PC72592_ST25.txt” created on Jan. 29, 2021 and having a size of 34 KB. The sequence listing contained in this .txt file is part of the specification and is incorporated herein by reference in its entirety.

### FIELD OF THE INVENTION

The present invention relates to methods for purifying bacterial polysaccharides, in particular for removing impurities from cellular lysates of bacteria producing polysaccharides.

### BACKGROUND OF THE INVENTION

Bacterial polysaccharides, in particular capsular polysaccharides, are important immunogens found on the surface of bacteria involved in various bacterial diseases. This has led to them being an important component in the design of vaccines. They have proven useful in eliciting immune responses especially when linked to carrier proteins.

Bacterial polysaccharides are typically produced by fermentation of the bacteria (e.g. Streptococci (e.g., S. pneumoniae, S. pyogenes, S. agalactiae or Group C & G Streptococci), Staphylococci (e.g., Staphylococcus aureus), Haemophilus, (e.g., Haemophilus influenzae), Neisseria (e.g., Neisseria meningitidis), Escherichia, (e.g., Escherichia coli) and Klebsiella (e.g., Klebsiella pneumoniae).

Typically, bacterial polysaccharides are produced using batch culture in complex medium, fed batch culture or continuous culture.

There is a need for robust and efficacious purification processes that can be used in the large-scale production of bacterial polysaccharides post-fermentation.

There is also a need for a simplified purification process to reduce the soluble protein levels in bacterial lysates and eliminate inefficiencies of the current purification process to produce substantially purified bacterial saccharides suitable for incorporation into vaccines.

### SUMMARY OF THE INVENTION

This invention provides a method for purifying a saccharide derived from bacteria from a solution comprising said saccharide and contaminants following fermentation, wherein said method comprises the following steps: (a) acid hydrolysis; (b) a first ultrafiltration/diafiltration-(UFDF-1); (b) carbon filtration; (c) chromatography; and (d) a second ultrafiltration/diafiltration-(UFDF-2). In one embodiment, the method further comprises a flocculation step following the acid hydrolysis of step (a).

In another embodiment of the above methods, the bacteria is a gram positive bacteria. In one aspect, the bacteria is any one of Streptococcus, Staphylococcus, Enterococci, Bacillus, Corynebacterium, Listeria, Erysipelothrix, or Clostridium. In a further aspect, the bacteria is any one of Streptococcus pneumoniae, Streptococcus pyogenes, Streptococcus agalactiae, Group C & G Streptococcii or Staphylococcus aureus.

In another embodiment of the above methods, the bacteria is a gram negative bacteria. In one aspect, the bacteria is any one of Haemophilus, Neisseria, Escherichia or Klebsiella. In another aspect, the bacteria is Haemophilus influenzae, Neisseria meningitidis, Escherichia coli or Klebsiella pneumoniae.

### BRIEF DESCRIPTION OF THE DRAWINGS

FIG. 1 depicts SEC-HPLC chromatograms for post released Kₚ O—Ag in broth (top) and purified Kₚ O—Ag (bottom) for O1V1 variant.

FIG. 2 depicts SEC-HPLC chromatograms for post released Kₚ O—Ag in broth (top) and purified Kₚ O—Ag (bottom) for O1V2 variant.

FIG. 3 depicts SEC-HPLC chromatograms for post released Kₚ O—Ag in broth (top) and purified Kₚ O—Ag (bottom) for O2V1 variant.

FIG. 4 depicts SEC-HPLC chromatograms for post released Kₚ O—Ag in broth (top) and purified Kₚ O—Ag (bottom) for O2V2 variant.

### DETAILED DESCRIPTION

This invention provides a method for purifying a saccharide derived from bacteria from a solution comprising said saccharide and contaminants following fermentation, wherein said method comprises the following steps: (a) acid hydrolysis; (b) a first ultrafiltration/diafiltration-(UFDF-1); (b) carbon filtration; (c) chromatography; and (d) a second ultrafiltration/diafiltration-(UFDF-2).

In one embodiment, the method further comprises a flocculation step following the acid hydrolysis of step (a).

In another embodiment of the above methods, the chromatography of step (c) comprises IEX membrane chromatography or Hydrophobic Interaction Chromatography (HIC) or both.

In another embodiment of the above methods, the bacteria is a gram positive bacteria. In one aspect, the bacteria is any one of Streptococcus, Staphylococcus, Enterococci, Bacillus, Corynebacterium, Listeria, Erysipelothrix, or Clostridium. In a further aspect, the bacteria is any one of Streptococcus pneumoniae, Streptococcus pyogenes, Streptococcus agalactiae, Group C & G Streptococcii or Staphylococcus aureus.

In another embodiment of the above methods, the bacteria is a gram negative bacteria. In one aspect, the bacteria is any one of Haemophilus, Neisseria, Escherichia or Klebsiella. In another aspect, the bacteria is Haemophilus influenzae, Neisseria meningitidis, Escherichia coli or Klebsiella pneumoniae.

In a further aspect, the bacteria is Escherichia coli comprising a saccharide having a structure selected from any one of Formula O1, Formula O1A, Formula O1B, Formula O1C, Formula O2, Formula O3, Formula O4, Formula O4:K52, Formula O4:K6, Formula O5, Formula O5ab, Formula O5ac, Formula O6, Formula O6:K2; K13; K15, Formula O6:K54, Formula O7, Formula O8, Formula O9, Formula O10, Formula O11, Formula O12, Formula O13, Formula O14, Formula O15, Formula O16, Formula O17, Formula O18, Formula O18A, Formula O18ac, Formula O18A1, Formula O18B, Formula O18B1, Formula O19, Formula O20, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186 or Formula O187.

In another aspect, the bacteria is Klebsiella pneumoniae comprising a saccharide having a structure selected from any one of Formula K.O1.1, Formula K.O1.2, Formula K.O1.3, Formula K.O1.4, Formula K.O2.1, Formula K.O2.2, Formula K.O2.3, Formula K.O2.4, Formula K.O3, Formula K.O4, Formula K.O5, Formula K.O7, Formula K.O12 or Formula K.O8.

1. Purification process of bacterial polysaccharides

1.1 Starting Material

The methods of the invention can be used to purify bacterial polysaccharides from a solution comprising said polysaccharides together with contaminants.

1.1.1. Bacterial Cells

The sources of bacterial polysaccharide to be purified according to this invention are bacterial cells, in particular pathogenic bacteria.

Non-limiting examples of gram-positive bacteria for use according to this invention are Streptococcus (e.g., S. pneumoniae, S. pyogenes, S. agalactiae or Group C & G Streptococci), Staphylococcus (e.g., Staphylococcus aureus), Enterococci, Bacillus, Corynebacterium, Listeria, Erysipelothrix, and Clostridium. Non-limiting examples of gram-negative bacteria for use with this invention include Haemophilus, (e.g., Haemophilus influenzae), Neisseria (e.g., Neisseria meningitidis), Escherichia, (e.g., Escherichia coli) and Klebsiella (e.g., Klebsiella pneumoniae).

In an embodiment, the source of bacterial polysaccharides for use according to this invention is selected from the group consisting of Aeromonas hydrophila and other species (spp.); Bacillus anthracis; Bacillus cereus; Botulinum neurotoxin-producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerosporaphilippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio par ahaemo ly ticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

A polysaccharide desired for purification may be associated with a cellular component, such as a cell wall. Association with the cell wall means that the polysaccharide is a component of the cell wall itself, and/or is attached to the cell wall, either directly or indirectly via intermediary molecules, or is a transient coating of the cell wall (for example, certain bacterial strains exude capsular polysaccharides, also known in the art as ‘exopolysaccharides’).

In some embodiments, the polysaccharide extracted from the bacteria is a capsular polysaccharide, a sub-capsular polysaccharide, or a lipopolysaccharide. In a preferred embodiment, the polysaccharide is a capsular polysaccharide.

In an embodiment, the source of bacterial capsular polysaccharide is Escherichia coli. In a further embodiment, the source of bacterial capsular polysaccharide is an Escherichia coli part of the Enterovirulent Escherichia coli group (EEC Group) such as Escherichia coli-enterotoxigenic (ETEC), Escherichia coli—enteropathogenic (EPEC), Escherichia coli-O157:H7 enterohemorrhagic (EHEC), or Escherichia coli—enteroinvasive (EIEC). In an embodiment, the source of bacterial capsular polysaccharide is an Uropathogenic Escherichia coli (UPEC).

In another embodiment, the source of bacterial capsular polysaccharide is an Escherichia coli serotype selected from the group consisting of serotypes O157:H7, O26:H111, O111:H- and O103:H2. In an embodiment, the source of bacterial capsular polysaccharide is an Escherichia coli serotype selected from the group consisting of serotypes O6:K2:H1 and O18:K1:H7. In an embodiment, the source of bacterial capsular polysaccharide is an Escherichia coli serotype selected from the group consisting of serotypes O45:K1, O17:K52:H18, O19:H34 and O7:K1. In an embodiment, the source of bacterial capsular polysaccharide is an Escherichia coli serotype O104:H4. In an embodiment, the source of bacterial capsular polysaccharide is an Escherichia coli serotype O1:K12:H7. In an embodiment, the source of bacterial capsular polysaccharide is an Escherichia coli serotype O127:H6. In an embodiment, the source of bacterial capsular polysaccharide is an Escherichia coli serotype O139:H28. In an embodiment, the source of bacterial capsular polysaccharide is an Escherichia coli serotype O128:H2.

In another embodiment, the source of bacterial capsular polysaccharides is Neisseria meningitidis. In an embodiment the source of bacterial capsular polysaccharides is N. meningitidis serogroup A (MenA), N. meningitidis serogroup W135 (MenW135), N. meningitidis serogroup Y (MenY), N. meningitidis serogroup X (MenX) or N. meningitidis serogroup C (MenC). In an embodiment the source of bacterial capsular polysaccharides is N. meningitidis serogroup A (MenA). In an embodiment the source of bacterial capsular polysaccharides is N. meningitidis serogroup W135 (MenW135). In an embodiment the source of bacterial capsular polysaccharides is N. meningitidis serogroup Y (MenY). In an embodiment the source of bacterial capsular polysaccharides is N. meningitidis serogroup C (MenC). In an embodiment the source of bacterial capsular polysaccharides is N. meningitidis serogroup X (MenX).

In a further embodiment, the source of bacterial capsular polysaccharides is Klebsiella pneumoniae. In an embodiment the source of bacterial capsular polysaccharides is K. pneumoniae serogroup O1 (O1), K. pneumoniae serogroup O2 (O2), K. pneumoniae serogroup O2ac (O2ac), K. pneumoniae serogroup O3 (O3), K. pneumoniae serogroup O4 (O4), K. pneumoniae serogroup O5 (O5), K. pneumoniae serogroup O7 (O7), K. pneumoniae serogroup O8 (O8) or K. pneumoniae serogroup O9 (O9). In an embodiment the source of bacterial capsular polysaccharides is K. pneumoniae serogroup O1 (O1). In an embodiment the source of bacterial capsular polysaccharides is K. pneumoniae serogroup O2 (O2). In an embodiment the source of bacterial capsular polysaccharides is K. pneumoniae serogroup O2ac (O2ac). In an embodiment the source of bacterial capsular polysaccharides is K. pneumoniae serogroup O3 (O3). In an embodiment the source of bacterial capsular polysaccharides is K. pneumoniae serogroup O4 (O4). In an embodiment the source of bacterial capsular polysaccharides is K. pneumoniae serogroup O5 (O5). In an embodiment the source of bacterial capsular polysaccharides is K. pneumoniae serogroup O7 (O7). In an embodiment the source of bacterial capsular polysaccharides is K. pneumoniae serogroup O8 (O8). In an embodiment the source of bacterial capsular polysaccharides is K. pneumoniae serogroup O9 (O9).

1.1.2. Bacterial Cells Growth

Typically the polysaccharides are produced by growing the bacteria in a medium (e.g. a solid or preferably a liquid medium). The polysaccharides are then prepared by treating the bacterial cells.

Therefore in an embodiment, the starting material for methods of the present invention is a bacterial culture and preferably a liquid bacterial culture (e.g. a fermentation broth).

The bacterial culture is typically obtained by batch culture, fed batch culture or continuous culture (see e.g. WO 2007/052168 or WO 2009/081276). During continuous culture, fresh medium is added to a culture at a fixed rate and cells and medium are removed at a rate that maintains a constant culture volume.

The population of the organism is often scaled up from a seed vial to seed bottles and passaged through one or more seed fermentors of increasing volume until production scale fermentation volumes are reached.

1.1.3 Pre-Treatment of the Bacterial Cells in Order to Obtain the Starting Material

Generally, a small amount of polysaccharide is released into the culture medium during bacterial growth, and so the starting material may thus be the supernatant from a centrifuged bacterial culture. Typically, however, the starting material will be prepared by treating the bacteria themselves, such that the polysaccharide is released.

Optionally, after cell growth, the bacterial cells are deactivated. This is particularly the case when pathogenic bacteria are used. A suitable method for deactivation is for example treatment with phenol:ethanol, e.g. as described in Fattom et al. (1990) Infect Immun. 58(7):2367-74. In the below embodiments, the bacterial cells may be previously deactivated or not deactivated.

Polysaccharides can be released from bacteria by various methods, including chemical, physical or enzymatic treatment (see e.g.; WO2010151544, WO 2011/051917 or WO2007084856).

In an embodiment, the bacterial cells (deactivated or not deactivated) are treated in suspension in their original culture medium. The process may therefore start with the cells in suspension in their original culture medium.

In another embodiment the bacterial cells are centrifuged prior to release of capsular polysaccharide. The process may therefore start with the cells in the form of a wet cell paste. Alternatively, the cells are treated in a dried form. Typically, however, after centrifugation the bacterial cells are resuspended in an aqueous medium that is suitable for the next step in the process, e.g. in a buffer or in distilled water. The cells may be washed with this medium prior to re-suspension.

In an embodiment, the bacterial cells (e.g. in suspension in their original culture medium, in the form of a wet cell paste, in a dried form or resuspended in an aqueous medium after centrifugation) are treated with a lytic agent. A “lytic agent” is any agent that aids in cell wall breakdown. In an embodiment, the lytic agent is a detergent. As used herein, the term “detergent” refers to any anionic or cationic detergent capable of inducing lysis of bacterial cells. Representative examples of such detergents for use within the methods of the present invention include deoxycholate sodium (DOC), N-lauryl sarcosine (NLS), chenodeoxycholic acid sodium, and saponins (see WO 2008/118752 pages 13 lines 14 to page 14 line 10). In one embodiment of the present invention, the lytic agent used for lysing bacterial cells is DOC.

In an embodiment, the lytic agent is a non-animal derived lytic agent. In one embodiment, the non-animal derived lytic agent is selected from the group consisting of decanesulfonic acid, tert-octylphenoxy 5 poly(oxyethylene)ethanols (e.g. IGEPAL CA-630, CAS #: 9002-93-1, available from Sigma Aldrich, St. Louis, Mo.), octylphenol ethylene oxide condensates (e.g. TRITON X-100, available from Sigma Aldrich, St. Louis, Mo.), N-lauryl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate, and cholate. In an embodiment, the non-animal derived lytic agent is NLS.

In an embodiment, the bacterial cells (e.g. in suspension in their original culture medium, in the form of a wet cell paste, in a dried form or resuspended in an aqueous medium after centrifugation) are enzymatically treated such that the polysaccharide is released. In an embodiment, the bacterial cells are treated by an enzyme selected from the group consisting of lysostaphin, mutanolysin β-N-acetylglucosaminidase and a combination of mutanolysin and β-N-acetylglucosaminidase. These act on the bacterial peptidoglycan to release the capsular saccharide for use with the invention but also lead to release of the group-specific carbohydrate antigen. In an embodiment, the bacterial cells are treated by a type II phosphodiesterase (PDE2).

Optionally, after polysaccharide release, the enzyme(s) is/are deactivated. A suitable method for deactivation is, for example, heat treatment or acidic treatment.

In an embodiment, the bacterial cells (e.g. in suspension in their original culture medium, in the form of a wet cell paste, in a dried form or resuspended in an aqueous medium after centrifugation) are autoclaved such that the polysaccharide is released.

In a further embodiment, the bacterial cells (e.g. in suspension in their original culture medium, in the form of a wet cell paste, in a dried form or resuspended in an aqueous medium after centrifugation) are chemically treated such that the polysaccharide is released. In such an embodiment, the chemical treatment can be, for example, hydrolysis using base or acid (see e.g. WO2007084856).

In an embodiment, the bacterial cells chemical treatment is base extraction (e.g., using sodium hydroxide). Base extraction can cleave the phosphodiester linkage between the capsular saccharide and the peptidoglycan backbone. In an embodiment, the base is selected from the group consisting of NaOH, KOH, LiOH, NaHC03, Na2C03, KzC03, KCN, Et3N, NH3, HzN2H2, NaH, NaOMe, NaOEt and KOtBu. After base treatment, the reaction mixture may be neutralised. This may be achieved by the addition of an acid. In an embodiment, after base treatment, the reaction mixture is neutralised by an acid selected from the group consisting of HCl, H₃PO₄, citric acid, acetic acid, nitrous acid, and sulfuric acid.

In an embodiment, the bacterial cells chemical treatment is acid treatment (e.g., sulfuric acid). In an embodiment, the acid is selected from the group consisting of HCl, H₃PO₄, citric acid, acetic acid, nitrous acid, and sulfuric acid. Following acid treatment, the reaction mixture may be neutralised. This may be achieved by the addition of a base. In an embodiment, after acid treatment, the reaction mixture is neutralised by a base selected from the group consisting of NaOH, KOH, LiOH, NaHC03, Na2C03, KzC03, KCN, Et3N, NH3, HzN2H2, NaH, NaOMe, NaOEt and KOtBu.

1.2 Flocculation

The methods of the invention comprise a flocculation step. The inventors have found that the process is quick and simple and results in a purified polysaccharide with low contamination.

Therefore, in the method of the invention, the solution obtained by any of the methods of section 1.1 above is treated by flocculation.

In the present invention, the term “flocculation” refers to a process wherein colloids come out of suspension in the form of floc or flake due to the addition of a flocculating agent.

The flocculation step comprises adding a “flocculating agent” to a solution comprising bacterial polysaccharides together with contaminants. In an embodiment, the contaminants comprise bacterial cell debris, bacterial cell proteins and nucleic acids. In an embodiment, the contaminants comprise bacterial cell proteins and nucleic acids.

As it will be further disclosed herebelow, the flocculation step may further include adjustment of the pH, either before or after the addition of the flocculating agent. In particular the solution may be acidified.

Furthermore, the addition of the flocculating agent and/or the adjustment of the pH may be performed at a temperature adjusted to a desirable level.

The following steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be held for some time to allow settling of the flocs prior to downstream processing.

In the present invention a “flocculating agent” refers to an agent being capable of allowing or promoting flocculation, in a solution comprising a polysaccharide of interest together with contaminants, by causing colloids and other suspended particles to aggregate in the form of floc or flake, while the polysaccharide of interest stays in solution.

In an embodiment of the present invention, the flocculating agent comprises a multivalent cation. In an embodiment, the flocculating agent is a multivalent cation. In a preferred embodiment said multivalent cation is selected from the group consisting of aluminium, iron, calcium and magnesium. In an embodiment the flocculating agent is a mixture of at least two multivalent cations selected from the group consisting of aluminium, iron, calcium and magnesium. In an embodiment the flocculating agent is a mixture of at least three multivalent cations selected from the group consisting of aluminium, iron, calcium and magnesium. In an embodiment the flocculating agent is a mixture of four multivalent cations consisting of aluminium, iron, calcium and magnesium.

In an embodiment, the flocculating agent comprises an agent selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is polyethylenimine (PEI). In an embodiment, the flocculating agent comprises alum. In an embodiment, the flocculating agent is alum. In an embodiment, the flocculating agent comprises potassium alum. In an embodiment, the flocculating agent is potassium alum. In an embodiment, the flocculating agent comprises sodium alum. In an embodiment, the flocculating agent is sodium alum. In an embodiment, the flocculating agent comprises ammonium alum. In an embodiment, the flocculating agent is ammonium alum.

In an embodiment, the flocculating agent is a mixture of agents (e.g. two, three or four agents) selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

In an embodiment, the flocculating agent is a mixture of two agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is a mixture of at least three agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

In an embodiment, the flocculating agent comprises an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).

The concentration of flocculating agent may depend on the agent(s) used, the polysaccharide of interest and the parameter of the flocculation step (e.g. temperature).

In embodiments where the flocculating agent comprises or is alum, a concentration of flocculating agent of between about 0.1 and 20% (w/v) can be used. Preferably through a concentration of flocculating agent of between about 0.5 and 10% (w/v) is used. Even more preferably a concentration of flocculating agent of between about 1 and 5% (w/v) is used. Any number within any of the above ranges is contemplated as an embodiment of the disclosure.

In an embodiment, a concentration of flocculating agent of about 0.1% (w/v), about 0.25% (w/v), about 0.5% (w/v), about 1.0% (w/v), about 1.5% (w/v), about 2.0% (w/v), about 2.5% (w/v), about 3.0% (w/v), about 3.5% (w/v), about 4.0% (w/v), about 4.5% (w/v), about 5.0% (w/v), about 5.5% (w/v), about 6.0% (w/v), about 6.5% (w/v), about 7.0% (w/v), about 7.5% (w/v), about 8.0% (w/v), about 8.5% (w/v), about 9.0% (w/v), about 9.5% (w/v) or about 10% (w/v) is used. In an embodiment, a concentration of flocculating agent of about 10.5% (w/v), about 11.0% (w/v), about 11.5% (w/v), about 12.0% (w/v), about 12.5% (w/v), about 13.0% (w/v), about 13.5% (w/v), about 14.0% (w/v), about 14.5% (w/v), about 15.0% (w/v), about 15.5% (w/v), about 16.0% (w/v), about 16.5% (w/v), about 17.0% (w/v), about 17.5% (w/v), about 18.0% (w/v), about 18.5% (w/v), about 19.0% (w/v), about 19.5% (w/v) or about 20.0% (w/v) is used. In an embodiment, a concentration of flocculating agent of about 0.5% (w/v), about 1.0% (w/v), about 1.5% (w/v), about 2.0% (w/v), about 2.5% (w/v), about 3.0% (w/v), about 3.5% (w/v), about 4.0% (w/v), about 4.5% (w/v) or about 5.0% (w/v) is used. In an embodiment, a concentration of flocculating agent of about 1.0% (w/v), about 1.5% (w/v), about 2.0% (w/v), about 2.5% (w/v), about 3.0% (w/v), about 3.5% (w/v) or about 4.0% (w/v) is used.

In some embodiments of the present invention, the flocculating agent is added over a certain period of time. In some embodiments of the present invention, the flocculating agent is added over a period of between a few seconds (e.g. 1 to 10 seconds) and about one month. In some embodiments the flocculating agent is added over a period of between about 2 seconds and about two weeks. In some embodiments of the present invention, the flocculating agent is added over a period of between about 1 minute and about one week. In some embodiments the flocculating agent is added over a period of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore, in certain embodiments, the flocculating agent is added over a period of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

Preferably the flocculating agent is added over a period of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In certain embodiments the flocculating agent is added over a period of between about 15 minutes and about 3 hours. In certain embodiments the flocculating agent is added over a period of between about 30 minutes and about 120 minutes.

Any number within any of the above ranges is contemplated as an embodiment of the disclosure.

In an embodiment, the flocculating agent may be added over a period of about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.

In an embodiment, the flocculating agent is added without agitation. In another embodiment, the flocculating agent is added under agitation. In another embodiment, the flocculating agent is added under gentle agitation. In another embodiment, the flocculating agent is added under vigorous agitation.

The inventors have further surprisingly noted that the flocculation is improved when performed at an acidic pH.

Therefore, in an embodiment of the present invention, the flocculation step is performed at a pH below 7.0, 6.0, 5.0 or 4.0. In a particular embodiment of the present invention, the flocculation step is performed at a pH between 7.0 and 1.0. In an embodiment, the flocculation step is performed at a pH between 5.5 and 2.5, 5.0 and 2.5, 4.5 and 2.5, 4.0 and 2.5, 5.5 and 3.0, 5.0 and 3.0, 4.5 and 3.0, 4.0 and 3.0, 5.5 and 3.5, 5.0 and 3.5, 4.5 and 3.5 or 4.0 and 3.5. In an embodiment, the flocculation step is performed at a pH of about 5.5, about 5.0, about 4.5, about 4.0, about 3.5, about 3.0, about 2.5, about 2.0, about 1.5 or about 1.0. In an embodiment, the flocculation step is performed at a pH of about 4.0, about 3.5, about 3.0 or about 2.5. In an embodiment, the flocculation step is performed at a pH of about 3.5. Any number within any of the above ranges is contemplated as an embodiment of the disclosure.

In an embodiment, said acidic pH is obtained by acidifying the solution obtained by any of the method of section 1.1 above or further clarified as disclosed at section 1.2 with an acid. In an embodiment said acid is selected from the group consisting of HCl, H₃PO₄, citric acid, acetic acid, nitrous acid, and sulfuric acid. In an embodiment said acid is an amino acid. In an embodiment said acid is an amino acid selected from the group consisting of glycine, alanine and glutamate. In an embodiment said acid is HCl (hydrochloric acid). In an embodiment said acid is sulfuric acid.

In an embodiment, the acid is added is without agitation. Preferably, the acid is added is under agitation. In an embodiment, the acid is added under gentle agitation. In an embodiment, the acid is added under vigorous agitation.

In some embodiments of the present invention, following the addition of the flocculating agent (and the optional acidification), the solution is hold for some time to allow settling of the flocs prior to downstream processing.

In some embodiments of the present invention, the flocculation step is performed with a settling time of between a few seconds (e.g. 2 to 10 seconds) to about 1 minute. Preferably the settling time is at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155 or at least about 160 minutes. Preferably the settling time is less than a week, however the settling time maybe longer.

Therefore in certain embodiments, the settling time is between about 1, about 2, about 3, about 4, about 5, about 6, about 7, about 8, about 9, about 10, about 15, about 20, about 25, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380, about 1440 minute(s), about two days, about three days, about four days, about five days or about six days and 1 week.

In some embodiments of the present invention, the settling time is between a few seconds (e.g. 1 to 10 seconds) and about one month. In some embodiments the settling time is between about 2 seconds and about two weeks. In some embodiments of the present invention, the settling time is between about 1 minute and about one week. In some embodiments the settling time is between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore in certain embodiments, the settling time is between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

Preferably the settling time is between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In certain embodiments the settling time is between about 15 minutes and about 3 hours. In certain embodiments the settling time is between about 30 minutes and about 120 minutes.

Any number within any of the above ranges is contemplated as an embodiment of the disclosure.

In certain embodiments the settling time is about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.

Preferably the settling time is between about 5, about 10, about 15, about 20, about 25, about 30, about 60, about 90, about 120, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 or about 1440 minute(s) and two days. In certain embodiments the settling time is between about 5 minutes and about one day. In certain embodiments the settling time is between about 5 minutes and about 120 minutes.

The settling time may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.

Any number within any of the above ranges is contemplated as an embodiment of the disclosure.

In an embodiment, the optional settling step is conducted without agitation. In an embodiment, the optional settling step is conducted under agitation. In another embodiment, the optional settling step is conducted under gentle agitation. In another embodiment, the optional settling step is conducted under vigorous agitation.

In an embodiment of the present invention, the addition of the flocculating agent, the settling of the solution and/or the adjustment of the pH is performed at a temperature between about 4° C. and about 30° C. In an embodiment, the addition of the flocculating agent, the settling of the solution and/or the adjustment of the pH is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C. In an embodiment, the addition of the flocculating agent, the settling of the solution and/or the adjustment of the pH is performed at a temperature of about 20° C. The inventors have surprisingly noted that the flocculation can be further improved when performed at elevated temperature. Therefore, in a particular embodiment of the present invention, the addition of the flocculating agent, the settling of the solution and/or the adjustment of the pH is performed at temperature between about 30° C. to about 95° C. In an embodiment, the addition of the flocculating agent, the settling of the solution and/or the adjustment of the pH is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. In an embodiment, the addition of the flocculating agent, the settling of the solution and/or the adjustment of the pH is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the addition of the flocculating agent, the settling of the solution and/or the adjustment of the pH is performed at a temperature of about 50° C.

Any number within any of the above ranges is contemplated as an embodiment of the disclosure.

In an embodiment, the addition of the flocculating agent is performed at any of the above mentioned temperatures.

In an embodiment, the settling of the solution after the addition of the flocculating agent is performed at any of the above mentioned temperatures.

In an embodiment, the adjustment of the pH is performed at any of the above mentioned temperatures.

In an embodiment, the addition of the flocculating agent and the settling of the solution after the addition of the flocculating agent are performed at any of the above mentioned temperatures.

In an embodiment, the addition of the flocculating agent and the adjustment of the pH are performed at any of the above mentioned temperatures.

In an embodiment, the addition of the flocculating, the settling of the solution after the addition of the flocculating agent and the adjustment of the pH are performed at any of the above mentioned temperatures.

In an embodiment, the flocculation step comprises adding a flocculating agent (as disclosed above) without pH adjustment.

In an embodiment, the flocculation step comprises adding a flocculating agent and settling the solution (as disclosed above), without pH adjustment.

In an embodiment, the flocculation step comprises adding a flocculating agent, adjusting the pH and settling the solution (as disclosed above). In an embodiment, the flocculating agent is added before adjusting the pH. In another embodiment, the pH is adjusted before adding the flocculating agent.

In an embodiment, the flocculation step comprises adding a flocculating agent, settling the solution and adjusting the pH (as disclosed above). In an embodiment, the addition of flocculating agent and settling of the solution is conducted before adjusting the pH. In another embodiment, the pH is adjusted before adding the flocculating agent and settling the solution. In an embodiment, the addition of the flocculating agent and adjusting the pH is conducted before settling the solution. In another embodiment, the pH is adjusted before adding the flocculating agent and settling the solution.

In an embodiment, the flocculation step comprises adding a flocculating agent, adjusting the pH and adjustment of the temperature (as disclosed above).

These steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be hold for some time to allow settling of the flocs prior to downstream processing.

### 1.3. Solid/Liquid Separation

The flocculated material can be separated from the polysaccharide of interest by any suitable solid/liquid separation method.

Therefore in an embodiment of the present invention, after flocculation, the suspension (as obtained at section 1.2 above) is clarified by decantation, sedimentation, filtration or centrifugation. In an embodiment the polysaccharide-containing solution is then collected for storage and/or additional processing.

In an embodiment of the present invention, after flocculation, the suspension (as obtained at section 1.2 above) is clarified by decantation. Decanters are used to separate liquids where there is a sufficient difference in density between the liquids for the floc to settle. In an operating decanter there will be three distinct zones: clear heavy liquid, separating dispersed liquid (the dispersion zone), and clear light liquid. To produce a clean solution, a small amount of solution must generally be left in the container. Decanters can be designed for continuous operation.

In an embodiment of the present invention, after flocculation, the suspension (as obtained at section 1.2 above) is clarified by sedimentation (settling). Sedimentation is the separation of suspended solid particles from a liquid mixture by gravity settling into a clear fluid and a slurry of higher solids content. Sedimentation can be done in a thickener, in a clarifier or in a classifier. Since thickening and clarification are relatively cheap processes when used for the treatment of large volumes of liquid, they can be used for pre-concentration of feeds to filtering.

In an embodiment of the present invention, after flocculation, the suspension (as obtained at section 1.2 above) is clarified by centrifugation. In an embodiment said centrifugation is continuous centrifugation. In an embodiment said centrifugation is bucket centrifugation. In an embodiment the polysaccharide-containing supernatant is then collected for storage and/or additional processing.

In some embodiments the suspension is centrifuged at about 1,000 g about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g. In some embodiments the suspension is centrifuged at about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g or about 25,000 g.

In some embodiments the suspension is centrifuged between about 5,000 g and about 25,000 g. In some embodiments the suspension is centrifuged between about 8,000 g and about 20,000 g. In some embodiments the suspension is centrifuged between about 10,000 g and about 15,000 g. In some embodiments the suspension is centrifuged between about 10,000 g and about 12,000 g.

Any number within any of the above ranges is contemplated as an embodiment of the disclosure.

In some embodiments the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes. Preferably the centrifugation time is less than 24 hours.

Therefore in certain embodiments, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320 or about 1380 minutes and 1440 minutes.

Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes. In certain embodiments the suspension is centrifuged during between about 5 minutes and about 3 hours. In certain the suspension is centrifuged during between about 5 minutes and about 120 minutes.

The suspension may be centrifuged during between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes or about 155 minutes and about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes or about 55 minutes and about 60 minutes.

The suspension may be centrifuged during about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 minutes or about 1440 minutes.

The suspension may be centrifuged during about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes, about 55 minutes or about 60 minutes.

Any number within any of the above ranges is contemplated as an embodiment of the disclosure.

In an embodiment of the present invention, centrifugation is continuous centrifugation. In said embodiment, the feed rate can be of between of 50-5000 ml/min, 100-4000 ml/min, 150-3000 ml/min, 200-2500 ml/min, 250-2000 ml/min, 300-1500 ml/min, 300-1000 ml/min, 200-1000 ml/min, 200-1500 ml/min, 400-1500 ml/min, 500-1500 ml/min, 500-1000 ml/min, 500-2000 ml/min, 500-2500 ml/min or 1000-2500 ml/min.

In an embodiment, the feed rate can be of about 10, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1650 about 1700, about 1800, about 1900, about 2000, about 2100, about 2200, about 2300, about 2400, about 2500, about 2600, about 2700, about 2800, about 2900, about 3000, about 3250, about 3500, about 3750 about 4000, about 4250, about 4500 or about 5000 ml/min.

In an embodiment of the present invention, after flocculation, the suspension (as obtained at section 1.2 above) is clarified by filtration. In filtration, suspended solid particles in a liquid are removed by passing the mixture through a porous medium that retains particles and passes the clear filtrate. Filtration is performed on screens by gravity or on filters by vacuum, pressure or centrifugation. The solid can be retained on the surface of the filter medium, which is cake filtration, or captured within the filter medium, which is depth filtration. In an embodiment, after flocculation, the suspension (as obtained at section 1.2 above) is clarified by microfiltration. In an embodiment, microfiltration is tangential microfiltration. In another embodiment, microfiltration is dead-end filtration (perpendicular filtration). In an embodiment, microfiltration is dead-end filtration wherein diatomaceous earth (DE), also known as DE diatomite, is used as a filter aid to facilitate and enhance the efficiency of the solid/liquid separation. Therefore in an embodiment, after flocculation, the suspension (as obtained at section 1.2 above) is clarified by dead-end microfiltration comprising diatomaceous earth (DE). DE can be impregnated (or incorporated) into to the dead-end filter as an integral part of the depth filter.

In another format, the DE can be added to the flocculated solution (as obtained after section 1.2) in powder form. In the later case, the DE treated flocculated solution can be further clarified by depth filtration.

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a microfiltration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.

In an embodiment, the solution is treated by a microfiltration step wherein the filter has a nominal retention rating of about 0.45 micron.

In an embodiment, the solution is treated by a microfiltration step wherein the filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².

In an embodiment, the solution is treated by a microfiltration step wherein the filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a microfiltration step wherein the filter has a filter capacity of between 100-1500 L/m², 200-1500 L/m², 300-1500 L/m², 400-1500 L/m², 500-1500 L/m², 750-1500 L/m² or 1000-1500 L/m².

In an embodiment, the solution is treated by a microfiltration step wherein the filter has a filter capacity of between 100-1250 L/m², 200-1250 L/m², 300-1250 L/m², 400-1250 L/m², 500-1250 L/m², 750-1250 L/m² or 1000-1250 L/m².

In an embodiment, the solution is treated by a microfiltration step wherein the filter has a filter capacity of between 100-1000 L/m², 200-1000 L/m², 300-1000 L/m², 400-1000 L/m², 500-1000 L/m² or 750-1000 L/m².

In an embodiment, the solution is treated by a microfiltration step wherein the filter has a filter capacity of between 100-750 L/m², 200-750 L/m², 300-750 L/m², 400-750 L/m² or 500-750 L/m².

In an embodiment, the solution is treated by a microfiltration step wherein the filter has a filter capacity of between 100-600 L/m², 200-600 L/m², 300-600 L/m², 400-600 L/m² or 400-600 L/m².

In an embodiment, the solution is treated by a microfiltration step wherein the filter has a filter capacity of between 100-500 L/m², 200-500 L/m², 300-500 L/m² or 400-500 L/m².

Any number within any of the above ranges is contemplated as an embodiment of the disclosure.

In an embodiment, the solution is treated by a microfiltration step wherein the filter has a filter capacity of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m².

The solid/liquid separation methods described above can be used in a standalone format or in combination of two in any order, or in combination of three in any order.

1.4 Filtration (e.g. Depth Filtration)

Once the solution has been treated by the flocculation step of section 1.2 above and/or by the solid/liquid separation step of section 1.3 above, the polysaccharide containing solution (e.g. the supernatant) can optionally be further clarified.

In an embodiment, the solution is filtrated, thereby producing a further clarified solution. In an embodiment, the filtration is applied directly to the solution obtained by any of the method of section 1.2 above. In an embodiment, the filtration is applied to the solution further clarified by the solid/liquid separation step as described at section 1.3 above.

In an embodiment, the solution is treated by a filtration step selected from the group consisting of depth filtration, filtration through activated carbon, size filtration, diafiltration and ultrafiltration. In an embodiment, the solution is treated by a diafiltration step, particularly by tangential flow filtration. In an embodiment, the solution is treated by a depth filtration step.

Depth filters use a porous filtration medium to retain particles throughout the medium, rather than just on the surface of the medium. Due to the tortuous and channel-like nature of the filtration medium, the particles are retained throughout the medium within its structure, as opposed to on the surface.

In an embodiment, the solution is treated by a depth filtration step wherein the depth filter design is selected from the group consisting of cassettes, cartridges, deep bed (e.g. sand filter) and lenticular filters.

In an embodiment, the solution is treated by a depth filtration step wherein the depth filter has a nominal retention range of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.

In an embodiment, the solution is treated by a depth filtration step wherein the depth filter has a nominal retention range of between about 0.01-75 micron, about 0.05-75 micron, about 0.1-75 micron, about 0.2-75 micron, about 0.3-75 micron, about 0.4-75 micron, about 0.5-75 micron, about 0.6-75 micron, about 0.7-75 micron, about 0.8-75 micron, about 0.9-75 micron, about 1-75 micron, about 1.25-75 micron, about 1.5-75 micron, about 1.75-75 micron, about 2-75 micron, about 3-75 micron, about 4-75 micron, about 5-75 micron, about 6-75 micron, about 7-75 micron, about 8-75 micron, about 9-75 micron, about 10-75 micron, about 15-75 micron, about 20-75 micron, about 25-75 micron, about 30-75 micron, about 40-75 micron or about 50-75 micron.

In an embodiment, the solution is treated by a depth filtration step wherein the depth filter has a nominal retention range of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.

In an embodiment, the solution is treated by a depth filtration step wherein the depth filter has a nominal retention range of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.

In an embodiment, the solution is treated by a depth filtration step wherein the depth filter has a nominal retention range of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.

In an embodiment, the solution is treated by a depth filtration step wherein the depth filter has a nominal retention range of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.

In an embodiment, the solution is treated by a depth filtration step wherein the depth filter has a nominal retention range of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.

In an embodiment, the solution is treated by a depth filtration step wherein the depth filter has a nominal retention range of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.

In an embodiment, the solution is treated by a depth filtration step wherein the depth filter has a nominal retention range of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.

In an embodiment, the solution is treated by a depth filtration step wherein the depth filter has a nominal retention range of between about 0.05-50 micron, 0.1-25 micron 0.2-10, micron 0.1-10 micron, 0.2-5 micron or 0.25-1 micron.

Any number within any of the above ranges is contemplated as an embodiment of the disclosure.

In an embodiment, the solution is treated by a depth filtration step wherein the depth filter has a filter capacity of 1-2500 L/m², 5-2500 L/m², 10-2500 L/m², 25-2500 L/m², 50-2500 L/m², 75-2500 L/m², 100-2500 L/m², 150-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a depth filtration step wherein the depth filter has a filter capacity of 1-1000 L/m², 5-1000 L/m², 10-1000 L/m², 25-1000 L/m², 50-1000 L/m², 75-1000 L/m², 100-1000 L/m², 150-1000 L/m², 200-1000 L/m², 300-1000 L/m², 400-1000 L/m², 500-1000 L/m² or 750-1000 L/m².

In an embodiment, the solution is treated by a depth filtration step wherein the depth filter has a filter capacity of 1-750 L/m², 5-750 L/m², 10-750 L/m², 25-750 L/m², 50-750 L/m², 75-750 L/m², 100-750 L/m², 150-750 L/m², 200-750 L/m², 300-750 L/m², 400-750 L/m² or 500-750 L/m².

In an embodiment, the solution is treated by a depth filtration step wherein the depth filter has a filter capacity of 1-500 L/m², 5-500 L/m², 10-500 L/m², 25-500 L/m², 50-500 L/m², 75-500 L/m², 100-500 L/m², 150-500 L/m², 200-500 L/m², 300-500 L/m² or 400-500 L/m².

In an embodiment, the solution is treated by a depth filtration step wherein the depth filter has a filter capacity of 1-400 L/m², 5-400 L/m², 10-400 L/m², 25-400 L/m², 50-400 L/m², 75-400 L/m², 100-400 L/m², 150-400 L/m², 200-400 L/m² or 300-400 L/m².

In an embodiment, the solution is treated by a depth filtration step wherein the depth filter has a filter capacity of 1-300 L/m², 5-300 L/m², 10-300 L/m², 25-300 L/m², 50-300 L/m², 75-300 L/m², 100-300 L/m², 150-300 L/m² or 200-300 L/m².

In an embodiment, the solution is treated by a depth filtration step wherein the depth filter has a filter capacity of 1-200 L/m², 5-200 L/m², 10-200 L/m², 25-200 L/m², 50-200 L/m², 75-200 L/m², 100-200 L/m² or 150-200 L/m².

In an embodiment, the solution is treated by a depth filtration step wherein the depth filter has a filter capacity of 1-100 L/m², 5-100 L/m², 10-100 L/m², 25-100 L/m², 50-100 L/m² or 75-100 L/m².

In an embodiment, the solution is treated by a depth filtration step wherein the depth filter has a filter capacity of 1-50 L/m², 5-50 L/m², 10-50 L/m² or 25-50 L/m².

Any whole number integer within any of the above ranges is contemplated as an embodiment of the disclosure.

In an embodiment, the solution is treated by a depth filtration step wherein the feed rate is between 1-1000 LMH (liters/m²/hour), 10-1000 LMH, 25-1000 LMH, 50-1000 LMH, 100-1000 LMH, 125-1000 LMH, 150-1000 LMH, 200-1000 LMH, 250-1000 LMH, 300-1000 LMH, 400-1000 LMH, 500-1000 LMH, 600-1000 LMH, 700-1000 LMH, 800-1000 LMH or 900-1000 LMH.

In an embodiment, the solution is treated by a depth filtration step wherein the feed rate is between 1-500 LMH, 10-500 LMH, 25-500 LMH, 50-500 LMH, 100-500 LMH, 125-500 LMH, 150-500 LMH, 200-500 LMH, 250-500 LMH, 300-500 LMH or 400-500 LMH.

In an embodiment, the solution is treated by a depth filtration step wherein the feed rate is between 1-400 LMH, 10-400 LMH, 25-400 LMH, 50-400 LMH, 100-400 LMH, 125-400 LMH, 150-400 LMH, 200-400 LMH, 250-400 LMH or 300-400 LMH.

In an embodiment, the solution is treated by a depth filtration step wherein the feed rate is between 1-250 LMH, 10-250 LMH, 25-250 LMH, 50-250 LMH, 100-250 LMH, 125-250 LMH, 150-250 LMH or 200-250 LMH.

Any number within any of the above ranges is contemplated as an embodiment of the disclosure.

In an embodiment, the solution is treated by a depth filtration step wherein the feed rate is about 1, about 2, about 5, about 10, about 25, about 50, about 60, about 70, about 80, about 90, about 100, about 110, about 120, about 130, about 140, about 150, about 160, about 170, about 180, about 190, about 200, about 210, about 220, about 230, about 240 about 250, about 260, about 270, about 280, about 290, about 300, about 310, about 320, about 330, about 340, about 350, about 360, about 370, about 380, about 390, about 400, about 425, about 450, about 475, about 500, about 525, about 550, about 575, about 600, about 650, about 700, about 750, about 800, about 850, about 900, about 950 or about 1000 LMH.

### 1.5 Optional Further Filtration

Once the solution has been treated by the filtration step of section 1.4 above, the solution obtained (i.e. the filtrate) can optionally be further clarified.

In an embodiment, the solution is subjected to microfiltration. In an embodiment, microfiltration is dead-end filtration (perpendicular filtration). In an embodiment, microfiltration is tangential microfiltration.

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a depth filtration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.

In an embodiment, the solution is treated by a microfiltration step wherein the filter has a nominal retention rating of about 0.45 micron.

In an embodiment, the solution is treated by a microfiltration step wherein the filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².

In an embodiment, the solution is treated by a microfiltration step wherein the filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².

In an embodiment, the solution is treated by a microfiltration step wherein the filter has a filter capacity of between 100-1500 L/m², 200-1500 L/m², 300-1500 L/m², 400-1500 L/m², 500-1500 L/m², 750-1500 L/m² or 1000-1500 L/m².

In an embodiment, the solution is treated by a microfiltration step wherein the filter has a filter capacity of between 100-1250 L/m², 200-1250 L/m², 300-1250 L/m², 400-1250 L/m², 500-1250 L/m², 750-1250 L/m² or 1000-1250 L/m².

In an embodiment, the solution is treated by a microfiltration step wherein the filter has a filter capacity of between 100-1000 L/m², 200-1000 L/m², 300-1000 L/m², 400-1000 L/m², 500-1000 L/m² or 750-1000 L/m².

In an embodiment, the solution is treated by a microfiltration step wherein the filter has a filter capacity of between 100-750 L/m², 200-750 L/m², 300-750 L/m², 400-750 L/m² or 500-750 L/m².

In an embodiment, the solution is treated by a microfiltration step wherein the filter has a filter capacity of between 100-600 L/m², 200-600 L/m², 300-600 L/m², 400-600 L/m² or 400-600 L/m².

In an embodiment, the solution is treated by a microfiltration step wherein the filter has a filter capacity of between 100-500 L/m², 200-500 L/m², 300-500 L/m² or 400-500 L/m².

Any number within any of the above ranges is contemplated as an embodiment of the disclosure.

In an embodiment, the solution is treated by a microfiltration step wherein the filter has a filter capacity of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m².

1.6 Ultrafiltration and/or Diafiltration

Once the solution has been filtered by any of the method of section 1.4 above and/or by the filtration step of section 1.5 above, the solution obtained (i.e. the filtrate) can optionally be further clarified by Ultrafiltration and/or Dialfiltration.

Ultrafiltration (UF) is a process for concentrating a dilute product stream. UF separates molecules in solution based on the membrane pore size or molecular weight cutoff (MWCO).

In an embodiment of the present invention, the solution (e.g. the filtrate obtained at section 1.5 or 1.6 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa. In an embodiment the molecular weight cut off of the membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.

In an embodiment the molecular weight cut off of the membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.

In an embodiment the molecular weight cut off of the membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.

In an embodiment the molecular weight cut off of the membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.

In an embodiment the molecular weight cut off of the membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.

In an embodiment, the concentration factor of the ultrafiltration step is from about 1.5 to 10. In an embodiment, the concentration factor is from about 2 to 8. In an embodiment, the concentration factor is from about 2 to 5.

In an embodiment, the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2, about 3, about 4, about 5, or about 6.

In an embodiment of the present invention, the solution (e.g. the filtrate obtained at section 1.4 or 1.5 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the group consisting of magnesium chloride, potassium chloride, sodium chloride and a combination thereof. In one particular embodiment, the salt is sodium chloride. In one embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM. In one particular embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM or about 300 mM.

In an embodiment, the replacement solution is a buffer solution. In an embodiment, the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate) Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).

In an embodiment, the diafiltration buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate). In an embodiment, the diafiltration buffer is a salt of citric acid (citrate). In an embodiment, the diafiltration buffer is a salt of succinic acid (Succinate). In an embodiment, said salt is a sodium salt. In an embodiment, said salt is a potassium salt.

In an embodiment, the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5. Any number within any of the above ranges is contemplated as an embodiment of the disclosure.

In an embodiment, the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0. In an embodiment, the pH of the diafiltration buffer is about 6.0, about 6.5, about 7.0, about 7.5, about 8.0, about 8.5 or about 9.0. In an embodiment, the pH of the diafiltration buffer is about 6.5, about 7.0 or about 7.5. In an embodiment, the pH of the diafiltration buffer is about 7.0.

In an embodiment, the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.

In an embodiment, the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.

In an embodiment, the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.

In an embodiment, the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.

In an embodiment, the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.

Any number within any of the above ranges is contemplated as an embodiment of the disclosure.

In an embodiment, the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.

In an embodiment, the concentration of the diafiltration buffer is about 10 mM.

In an embodiment, the replacement solution comprises a chelating agent. In an embodiment, the replacement solution comprises an alum chelating agent. In some embodiments, the chelating agent is selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.

In some embodiments, the chelating agent is selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), a salt of citric acid (citrate) and combinations of these.

In some embodiments, the chelating agent is Ethylene Diamine Tetra Acetate (EDTA).

In some embodiments, the chelating agent is a salt of citric acid (citrate). In some embodiments, the chelating agent is sodium citrate.

In general, the chelating agent is employed at a concentration from 1 to 500 mM. In an embodiment, the concentration of the chelating agent in the replacement solution is from 2 to 400 mM. In an embodiment, the concentration of the chelating agent in the replacement solution solution is from 10 to 400 mM. In an embodiment, the concentration of the chelating agent in the replacement solution is from 10 to 200 mM. In an embodiment, the concentration of the chelating agent in the replacement solution is from 10 to 100 mM. In an embodiment, the concentration of the chelating agent in the replacement solution is from 10 to 50 mM. In an embodiment, the concentration of the chelating agent in the replacement solution is from 10 to 30 mM.

In an embodiment, the concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, 30 about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.

In an embodiment, the concentration of the chelating agent in the replacement solution is about 15 mM, about 20 mM, about 25 mM, about 30 mM, about 35 mM, about 40 mM, about 45 mM or about 50 mM.

In an embodiment, the diafiltration buffer solution comprises a salt. In some embodiments, the salt is selected from the groups consisting of magnesium chloride, potassium chloride, sodium chloride and a combination thereof. In one particular embodiment, the salt is sodium chloride. In an embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250, about 300, about 350, about 400, about 450 or about 500 mM. In one particular embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.

In an embodiment of the present invention, the number of diavolumes is at least 5, 10, 15, 20, 25, 30, 35, 40, 45, or 50. In an embodiment of the present invention, the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100. In an embodiment of the present invention the number of diavolumes is about 5, about 6, about 7, about 8, about 9, about 10, about 11, about 12, about 13, about 14 or about 15.

In an embodiment of the present invention, the Ultrafiltration and Dialfiltration steps are performed at a temperature between about 20° C. to about 90° C. In an embodiment, the Ultrafiltration and Dialfiltration steps are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.

Any number within any of the above ranges is contemplated as an embodiment of the disclosure.

In an embodiment, the Ultrafiltration and Dialfiltration steps are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration and Dialfiltration step are performed at a temperature of about 50° C.

In an embodiment of the present invention, the Dialfiltration step is performed at temperature between about 20° C. to about 90° C. In an embodiment, the Dialfiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.

Any number within any of the above ranges is contemplated as an embodiment of the disclosure.

In an embodiment, Dialfiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Dialfiltration step is performed at a temperature of about 50° C.

In an embodiment of the present invention, the Ultrafiltration step is performed at temperature between about 20° C. to about 90° C. In an embodiment, the Ultrafiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Ultrafiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration step is performed at a temperature of about 50° C.

### 1.7 Activated Carbon Filtration

Once the solution has been treated by the flocculation step of section 1.2 above, the solution containing the polysaccharide can optionally be further clarified by an activated carbon filtration step.

In an embodiment, the solution of section 1.2 further treated by the solid/liquid separation step of section 1.3 (e.g. the supernatant) is further clarified by an activated carbon filtration step. In an embodiment, the solution further filtered by any of the method of section 1.4 above and/or by the filtration step of section 1.5 above is further clarified by an activated carbon filtration step. In an embodiment, the solution further clarified by an Ultrafiltration and/or Dialfiltration step of section 1.6 above is further clarified by an activated carbon filtration step.

A step of activated carbon filtration allows for further removing host cell impurities such as proteins and nucleic acids as well as colored impurities (see WO2008/118752).

In an embodiment, activated carbon (also named active charcoal) is added to the solution in an amount sufficient to absorb the majority of the proteins and nucleic acids contaminants, and then removed once the contaminants have been adsorbed onto activated carbon. In an embodiment the activated carbon is added in the form of a powder, as a granular carbon bed, as a pressed carbon block or extruded carbon block (see e.g. Norit active charcoal). In an embodiment, the activated carbon is added in an amount of about 0.1 to 20% (weight volume), 1 to 15% (weight volume), 1 to 10% (weight volume), 2 to 10% (weight volume), 3 to 10% (weight volume), 4 to 10% (weight volume), 5 to 10% (weight volume), 1 to 5% (weight volume) or 2 to 5% (weight volume). The mixture is then stirred and left to stand. In an embodiment, the mixture is left to stand for about 5, 10, 15, 20, 30, 45, 60, 90, 120, 180, 240 minutes or more. The activated carbon is then removed. The activated carbon can be removed for example by centrifugation or filtration.

In a preferred embodiment, the solution is filtered through activated carbon immobilized in a matrix. The matrix may be any porous filter medium permeable for the solution. The matrix may comprise a support material and/or a binder material. The support material may be a synthetic polymer or a polymer of natural origin. Suitable synthetic polymers may include polystyrene, polyacrylamide and polymethyl methacrylate, while polymers of natural origin may include cellulose, polysaccharide and dextran, agarose. Typically, the polymer support material is in the form of a fibre network to provide mechanical rigidity. The binder material may be a resin. The matrix may have the form of a membrane sheet. In an embodiment, the activated carbon immobilized in the matrix is in the form of a flow-through carbon cartridge. A cartridge is a self-contained entity containing powdered activated carbon immobilized in the matrix and prepared in the form of a membrane sheet. The membrane sheet may be captured in a plastic permeable support to form a disc.

Alternatively, the membrane sheet may be spirally wound. To increase filter surface area, several discs may be stacked upon each other. In particular, the discs stacked upon each other have a central core pipe for collecting and removing the carbon-treated sample from the filter. The configuration of stacked discs may be lenticular.

The activated carbon in the carbon filter may be derived from different raw materials, e.g. peat, lignite, wood or coconut shell.

Any process known in the art, such as steam or chemical treatment, may be used to activate carbon (e.g. wood-based phosphoric acid-activated carbon).

In the present invention, activated carbon immobilized in a matrix may be placed in a housing to form an independent filter unit. Each filter unit has its own in-let and out-let for the solution to be purified. Examples of filter units that are usable in the present invention are the carbon cartridges from Cuno Inc. (Meriden, USA) or Pall Corporation (East Hill, USA). In particular, CUNO zetacarbon filters are suitable for use in the invention. These carbon filters comprise a cellulose matrix into which activated carbon powder is entrapped and resin-bonded in place.

In an embodiment, the activated carbon filter disclosed above has a nominal micron rating of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.

In an embodiment, the activated carbon filter disclosed above has a nominal micron rating of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.

In an embodiment, the activated carbon filter disclosed above has a nominal micron rating of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.

In an embodiment, the activated carbon filter disclosed above has a nominal micron rating of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.

In an embodiment, the activated carbon filter disclosed above has a nominal micron rating of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.

In an embodiment, the activated carbon filter disclosed above has a nominal micron rating of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.

In an embodiment, the activated carbon filter disclosed above has a nominal micron rating of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.

In an embodiment, the activated carbon filter disclosed above has a nominal micron rating of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.

In an embodiment, the activated carbon filter disclosed above has a nominal micron ratings of between about 0.05-50 micron, 0.1-25 micron 0.2-10, micron 0.1-10 micron, 0.2-5 micron or 0.25-1 micron.

Any number within any of the above ranges is contemplated as an embodiment of the disclosure.

In an embodiment, the activated carbon filtration step is conducted at a feed rate of between 1-500 LMH, 10-500 LMH, 15-500 LMH, 20-500 LMH, 25-500 LMH, 30-500 LMH, 40-500 LMH, 50-500 LMH, 100-500 LMH, 125-500 LMH, 150-500 LMH, 200-500 LMH, 250-500 LMH, 300-500 LMH or 400-500 LMH.

In an embodiment, the activated carbon filtration step is conducted at a feed rate of between 1-200 LMH, 10-200 LMH, 15-200 LMH, 20-200 LMH, 25-200 LMH, 30-200 LMH, 40-200 LMH, 50-200 LMH, 100-200 LMH, 125-200 LMH or 150-200 LMH.

In an embodiment, the activated carbon filtration step is conducted at a feed rate of between 1-150 LMH, 10-150 LMH, 15-150 LMH, 20-150 LMH, 25-150 LMH, 30-150 LMH, 40-150 LMH, 50-150 LMH, 100-150 LMH or 125-150 LMH.

In an embodiment, the activated carbon filtration step is conducted at a feed rate of between 1-100 LMH, 10-100 LMH, 15-100 LMH, 20-100 LMH, 25-100 LMH, 30-100 LMH, 40-100 LMH, or 50-100 LMH.

In an embodiment, the activated carbon filtration step is conducted at a feed rate of between 1-75 LMH, 5-75 LMH, 10-75 LMH, 15-75 LMH, 20-75 LMH, 25-75 LMH, 30-75 LMH, 35-75 LMH, 40-75 LMH, 45-75 LMH, 50-75 LMH, 55-75 LMH, 60-75 LMH, 65-75 LMH, or 70-75 LMH.

In an embodiment, the activated carbon filtration step is conducted at a feed rate of between 1-50 LMH, 5-50 LMH, 7-50 LMH, 10-50 LMH, 15-50 LMH, 20-50 LMH, 25-50 LMH, 30-50 LMH, 35-50 LMH, 40-50 LMH or 45-50 LMH.

Any whole number integer within any of the above ranges is contemplated as an embodiment of the disclosure.

In an embodiment, the activated carbon filtration step is conducted at a feed rate of about 1, about 2, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 300, about 350, about 400, about 450, about 500, about 550, about 600, about 700, about 800, about 900, about 950 or about 1000 LMH.

In an embodiment, the solution is treated by an activated carbon filter wherein the filter has a filter capacity of between 5-1000 L/m², 10-750 L/m², 15-500 L/m², 20-400 L/m², 25-300 L/m², 30-250 L/m², 40-200 L/m² or 30-100 L/m².

Any number within any of the above ranges is contemplated as an embodiment of the disclosure.

In an embodiment, the solution is treated by an activated carbon filter wherein the filter has a filter capacity of about 5, about 10, about 15, about 20, about 25, about 30, about 35, about 40, about 45, about 50, about 55, about 60, about 65, about 70, about 75, about 80, about 85, about 90, about 100, about 125, about 150, about 175, about 200, about 225, about 250, about 275, about 300, about 400, about 500, about 600, about 700, about 800, about 900, or about 1000 L/m².

If the content of contaminants is above the fixed threshold after a first activated carbon filtration step, the said step can be repeated. In an embodiment of the present invention, 1, 2, 3, 4, 5, 6, 7, 8, 9, or 10 activated carbon filtration step(s) are performed. In an embodiment of the present invention, 1, 2 or 3 activated carbon filtration step(s) are performed. In an embodiment of the present invention, 1 or 2 activated carbon filtration step(s) are performed.

In an embodiment, the solution is treated by activated carbon filters in series. In an embodiment, the solution is treated by 1, 2, 3, 4, 5, 6, 7, 8, 9, or 10 activated carbon filters in series. In an embodiment, the solution is treated by 2, 3, 4 or 5 activated carbon filters in series.

In an embodiment, the solution is treated by 2 activated carbon filters in series. In an embodiment, the solution is treated by 3 activated carbon filters in series. In an embodiment, the solution is treated by 4 activated carbon filters in series. In an embodiment, the solution is treated by 5 activated carbon filters in series.

In an embodiment the activated carbon filtration step is performed in a single pass mode.

In another embodiment the activated carbon filtration step is performed in recirculation mode. In said embodiment (recirculation mode) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles of activated carbon filtration are performed. In another embodiment 2, 3, 4, 5, 6, 7, 8, 9 or 10 cycles of activated carbon filtration are performed. In an embodiment, 2 or 3 cycles of activated carbon filtration are performed. In an embodiment, 2 cycles of activated carbon filtration are performed.

### 1.8 Optional Further Filtration

Once the solution has been treated by the activated carbon step of section 1.7 above, the obtained solution (i.e. the filtrate) can optionally be further filtered.

In an embodiment, the solution is subjected to microfiltration. In an embodiment, microfiltration is dead-end filtration (perpendicular filtration).

In an embodiment, the solution is treated by a microfiltration step wherein the filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.

In an embodiment, the solution is treated by a microfiltration step wherein the filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.

Any number within any of the above ranges is contemplated as an embodiment of the disclosure.

In an embodiment, the solution is treated by a microfiltration step wherein the filter has a nominal retention rating of about 0.01, about 0.05, about 0.1, about 0.2, about 0.3, about 0.4, about 0.45, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0 micron.

In an embodiment, the solution is treated by a microfiltration step wherein the filter has a nominal retention rating of about 0.2 micron.

In an embodiment, the solution is treated by a microfiltration step wherein the filter has a filter capacity of 100-6000 L/m², 200-6000 L/m², 300-6000 L/m², 400-6000 L/m², 500-6000 L/m², 750-6000 L/m², 1000-6000 L/m², 1500-6000 L/m², 2000-6000 L/m², 3000-6000 L/m² or 4000-6000 L/m².

In an embodiment, the solution is treated by a microfiltration step wherein the filter has a filter capacity of 100-4000 L/m², 200-4000 L/m², 300-4000 L/m², 400-4000 L/m², 500-4000 L/m², 750-4000 L/m², 1000-4000 L/m², 1500-4000 L/m², 2000-4000 L/m², 2500-4000 L/m², 3000-4000 L/m², 3000-4000 L/m² or 3500-4000 L/m².

In an embodiment, the solution is treated by a microfiltration step wherein the filter has a filter capacity of 100-3750 L/m², 200-3750 L/m², 300-3750 L/m², 400-3750 L/m², 500-3750 L/m², 750-3750 L/m², 1000-3750 L/m², 1500-3750 L/m², 2000-3750 L/m², 2500-3750 L/m², 3000-3750 L/m², 3000-3750 L/m² or 3500-3750 L/m².

In an embodiment, the solution is treated by a microfiltration step wherein the filter has a filter capacity of 100-1250 L/m², 200-1250 L/m², 300-1250 L/m², 400-1250 L/m², 500-1250 L/m², 750-1250 L/m² or 1000-1250 L/m².

Any number within any of the above ranges is contemplated as an embodiment of the disclosure.

In an embodiment, the solution is treated by a microfiltration step wherein the filter has a filter capacity of about 100, about 200, about 300, about 400, about 550, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5250, about 5500, about 5750 or about 6000 L/m².

### 1.9 Hydrophobic Interaction Chromatography (HIC)

This unit operation removes any impurities that had hydrophobic characteristics, such as residual lipid polysaccharides (endotoxins) left from the former purification steps.

Once the solution has been treated by the flocculation step of section 1.2 above, the solution containing the polysaccharide can optionally be further purified by a HIC step.

In an embodiment, the solution of section 1.2 further treated by the solid/liquid separation step of section 1.3 (e.g. the supernatant) is further purified by a HIC step. In an embodiment, the solution further filtered by any of the method of section 1.5 above and/or by the filtration step of section 1.4 above is further purified by a HIC step. In an embodiment, the solution further clarified by an Ultrafiltration and/or Dialfiltration step of section 1.6 above is further purified by a HIC step. In an embodiment, the solution further clarified by activated carbon filtration step of section 1.7 is further purified by a HIC step.

In an embodiment, the solution further clarified by an Ultrafiltration and/or Dialfiltration step of section 1.6 above is further purified by an ion exchange membrane (IEX) filtration step and can then be further purified by a HIC step.

In an embodiment, the HIC step is conducted using an hydrophobic adsorbent selected from but not limited to the group consisting of a phenyl membrane, butyl-, phenyl-, and octyl-agarose, butyl-, phenyl-, ether-, polypropylenglycol- and hexyl-organic polymer resin.

In an embodiment, the hydrophobic adsorbent used in the HIC step is a phenyl membrane such as the SARTOBIND Phenyl membrane or CYTIVA's Phenyl Adsorber membrane.

In an embodiment, the hydrophobic adsorbent used in the HIC step is a the SARTOBIND Phenyl membrane.

In an embodiment, the material from the former step (for example the carbon filtrate) is treated with an equilibration buffer to obtain a running buffer comprising the material to be purified and a desired salt concentration. In an embodiment the equilibration buffer comprise a salt and the final salt concentration (i.e in the running buffer) is selected from about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9 or about 7.0M. In one embodiment, the running buffer has a pH between about 4.0 and about 8.0. In one embodiment, the pH of the running buffer is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0. In an embodiment, the equilibration buffer comprises a salt selected from ammonium sulfate (preferably at a final concentration in the running buffer of 0.5M-3.0M and pH 6.0±2.0), sodium phosphate (preferably at a final concentration in the running buffer of 0.5M-3.0M and pH 7.0±1.5), potassium phosphate (preferably at a final concentration in the running buffer of 0.5M-3.0M and pH 7.0±1.5), sodium sulfate (preferably at a final concentration in the running buffer of 0.1 M-0.75M and pH 6.0±2.0), sodium citrate (preferably at a final concentration in the running buffer of 0.1 M-1.5M and pH 6.0±2.0) or sodium chloride (preferably at a final concentration in the running buffer of 0.5M-5.0M and pH 7.0±1.5).

In an embodiment the equilibration buffer is comprises ammonium sulfate and the final salt concentration in the running buffer is comprised between about 1.0M and about 2.0M, preferably about 1.0, about 1.1, about 1.2, about 1.3, about 1.4, about 1.5, about 1.6, about 1.7, about 1.8, about 1.9, about 2.0 M).

In an embodiment, the hydrophobic adsorbent is equilibrated using the running buffer and the material to be purified in the running buffer is then ran through the column or membrane.

In an embodiment, the hydrophobic adsorbent is a phenyl membrane and the flow rate is comprised between about 0.1 and about 20 membrane volumes per min, about 0.1 and about 10 membrane volumes per min, about 0.2 and about 10 membrane volumes per min, about 0.2 and about 5 membrane volumes per min, about 0.1 and about 1 membrane volume per min. In an embodiment, the hydrophobic adsorbent is a phenyl membrane and the flow rate is comprised between about 0.1 and about 1.0 membrane volume per min, preferably about 0.1, about 0.2, about 0.3, about 0.4, about 0.5, about 0.6, about 0.7, about 0.8, about 0.9 or about 1.0 membrane volume per min.

In an embodiment, the HIC membrane is then rinsed with the running buffer and can also be further washed with water. The flow through effluent along with the buffer rinse was collected as HIC filtrate, and the water wash was also collected for analysis.

### 1.10 Ultrafiltration/Diafiltration

Once the solution has been treated by the HIC step of section 1.9 above and/or by the further filtration step of section 1.8 above, the obtained solution (i.e. the filtrate) can optionally be further clarified by Ultrafiltration and/or Dialfiltration.

In an embodiment of the present invention, the solution (e.g. obtained at section 1.9 or 1.8 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa. In an embodiment the molecular weight cut off of the membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.

In an embodiment the molecular weight cut off of the membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.

In an embodiment the molecular weight cut off of the membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.

In an embodiment the molecular weight cut off of the membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.

In an embodiment the molecular weight cut off of the membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.

In an embodiment, the concentration factor of the ultrafiltration step is from about 1.5 to about 10.0. In an embodiment, the concentration factor is from about 2.0 to about 8.0. In an embodiment, the concentration factor is from about 2.0 to about 5.0.

In an embodiment, the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2.0, about 3.0, about 4.0, about 5.0, or about 6.0.

In an embodiment of the present invention, the solution (e.g. the filtrate obtained at section 1.9 or 1.8 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the groups consisting of magnesium chloride, potassium chloride, sodium chloride and a combination thereof. In one particular embodiment, the salt is sodium chloride. In an embodiment, the replacement solution is sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250, about 300, about 350, about 400, about 450 or about 500 mM. In one particular embodiment, the replacement solution is sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM. In one particular embodiment, the replacement solution is sodium chloride at about 25, about 30, about 35, about 40, about 45, about 50, about 55, about 60, about 65, about 70, about 80, about 90 or about 100 mM.

In an embodiment, the replacement solution is a buffer solution. In an embodiment, the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate) Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).

In an embodiment, the diafiltration buffer is selected from the group consisting of a salt of acetic acid (acetate), a salt of citric acid (citrate), a salt of formic acid (formate), a salt of malic acid (malate), a salt of maleic acid (maleate), a salt of phosphoric acid (phosphate) and a salt of succinic acid (succinate). In an embodiment, the diafiltration buffer is a salt of citric acid (citrate). In an embodiment, the diafiltration buffer is a salt of succinic acid (succinate). In an embodiment, the diafiltration buffer is a salt of phosphoric acid (phosphate). In an embodiment, said salt is a sodium salt. In an embodiment, said salt is a potassium salt.

In an embodiment, the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5. Any number within any of the above ranges is contemplated as an embodiment of the disclosure.

In an embodiment, the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0. In an embodiment, the pH of the diafiltration buffer is about 6.0, about 6.5, about 7.0, about 7.5, about 8.0, about 8.5 or about 9.0. In an embodiment, the pH of the diafiltration buffer is about 6.5, about 7.0 or about 7.5. In an embodiment, the pH of the diafiltration buffer is about 6.0. In an embodiment, the pH of the diafiltration buffer is about 6.5. In an embodiment, the pH of the diafiltration buffer is about 7.0.

In an embodiment, the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.

In an embodiment, the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.

In an embodiment, the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.

In an embodiment, the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.

In an embodiment, the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.

Any number within any of the above ranges is contemplated as an embodiment of the disclosure.

In an embodiment, the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 11 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.

In an embodiment, the concentration of the diafiltration buffer is about 0.1 mM, about 0.2 mM, about 1 mM, about 5 mM, about 10 mM, about 15 mM, about 20 mM, about 25 mM, about 30 mM, about 40 mM, or about 50 mM. In an embodiment, the concentration of the diafiltration buffer is about 30 mM. In an embodiment, the concentration of the diafiltration buffer is about 25 mM. In an embodiment, the concentration of the diafiltration buffer is about 20 mM. In an embodiment, the concentration of the diafiltration buffer is about 15 mM. In an embodiment, the concentration of the diafiltration buffer is about 10 mM.

In an embodiment, the diafiltration buffer solution comprises a salt. In some embodiments, the salt is selected from the groups consisting of magnesium chloride, potassium chloride, sodium chloride and a combination thereof. In one particular embodiment, the salt is sodium chloride. In one particular embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.

In an embodiment of the present invention, the number of diavolumes is at least 5, 10, 15, 20, 25, 30, 35, 40, 45, or 50. In an embodiment of the present invention, the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100. In an embodiment of the present invention the number of diavolumes is about 5, about 6, about 7, about 8, about 9, about 10, about 11, about 12, about 13, about 14 or about 15.

In an embodiment of the present invention, the Ultrafiltration and Dialfiltration steps are performed at temperature between about 20° C. to about 90° C. In an embodiment, the Ultrafiltration and Dialfiltration steps are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, the Ultrafiltration and Dialfiltration steps are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration and Dialfiltration step are performed at a temperature of about 50° C.

In an embodiment of the present invention, the Dialfiltration step is performed at temperature between about 20° C. to about 90° C. In an embodiment, the Dialfiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Dialfiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Dialfiltration step is performed at a temperature of about 50° C.

In an embodiment of the present invention, the Ultrafiltration step is performed at temperature between about 20° C. to about 90° C. In an embodiment, the Ultrafiltration step is performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C. Any number within any of the above ranges is contemplated as an embodiment of the disclosure.

In an embodiment, Ultrafiltration step is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the Ultrafiltration step is performed at a temperature of about 50° C.

### 1.11 Homogenization/Sizing

A polysaccharide can become slightly reduced in size during the purification procedures.

In an embodiment, the purified solution of polysaccharide of the present invention (e.g. obtained by Ultrafiltration and/or Dialfiltration of section 1.10) is not sized.

In an embodiment, the polysaccharide can be homogenized by sizing techniques.

Mechanical or chemical sizing maybe employed. Chemical hydrolysis maybe conducted using for example acetic acid. Mechanical sizing maybe conducted using High Pressure Homogenization Shearing.

Therefore in an embodiment, the purified solution of polysaccharide obtained by Ultrafiltration and/or Dialfiltration of section 1.10 is sized to a target molecular weight.

As used herein, the term “molecular weight” of polysaccharide refers to molecular weight calculated for example by size exclusion chromatography (SEC) combined with multiangle laser light scattering detector (MALLS).

In some embodiments, the purified polysaccharide is sized to a molecular weight of between about 5 kDa and about 4,000 kDa. In other such embodiments, the purified polysaccharide is sized to a molecular weight of between about 10 kDa and about 4,000 kDa. In other such embodiments, the purified polysaccharide is sized to a molecular weight of between about 50 kDa and about 4,000 kDa. In further such embodiments, the polysaccharide the purified polysaccharide is sized to a molecular weight of between about 50 kDa and about 3,500 kDa; between about 50 kDa and about 3,000 kDa; between about 50 kDa and about 2,500 kDa; between about 50 kDa and about 2,000 kDa; between about 50 kDa and about 1,750 kDa; about between about 50 kDa and about 1,500 kDa; between about 50 kDa and about 1,250 kDa; between about 50 kDa and about 1,000 kDa; between about 50 kDa and about 750 kDa; between about 50 kDa and about 500 kDa; between about 100 kDa and about 4,000 kDa; between about 100 kDa and about 3,500 kDa; about 100 kDa and about 3,000 kDa; about 100 kDa and about 2,500 kDa; about 100 kDa and about 2,250 kDa; between about 100 kDa and about 2,000 kDa; between about 100 kDa and about 1,750 kDa; between about 100 kDa and about 1,500 kDa; between about 100 kDa and about 1,250 kDa; between about 100 kDa and about 1,000 kDa; between about 100 kDa and about 750 kDa; between about 100 kDa and about 500 kDa; between about 200 kDa and about 4,000 kDa; between about 200 kDa and about 3,500 kDa; between about 200 kDa and about 3,000 kDa; between about 200 kDa and about 2,500 kDa; between about 200 kDa and about 2,250 kDa; between about 200 kDa and about 2,000 kDa; between about 200 kDa and about 1,750 kDa; between about 200 kDa and about 1,500 kDa; between about 200 kDa and about 1,250 kDa; between about 200 kDa and about 1,000 kDa; between about 200 kDa and about 750 kDa; or between about 200 kDa and about 500 kDa. In further such embodiments, the polysaccharide the purified polysaccharide is sized to a molecular weight of between about 250 kDa and about 3,500 kDa; between about 250 kDa and about 3,000 kDa; between about 250 kDa and about 2,500 kDa; between about 250 kDa and about 2,000 kDa; between about 250 kDa and about 1,750 kDa; about between about 250 kDa and about 1,500 kDa; between about 250 kDa and about 1,250 kDa; between about 250 kDa and about 1,000 kDa; between about 250 kDa and about 750 kDa; between about 250 kDa and about 500 kDa; between about 300 kDa and about 4,000 kDa; between about 300 kDa and about 3,500 kDa; about 300 kDa and about 3,000 kDa; about 300 kDa and about 2,500 kDa; about 300 kDa and about 2,250 kDa; between about 300 kDa and about 2,000 kDa; between about 300 kDa and about 1,750 kDa; between about 300 kDa and about 1,500 kDa; between about 300 kDa and about 1,250 kDa; between about 300 kDa and about 1,000 kDa; between about 300 kDa and about 750 kDa; between about 300 kDa and about 500 kDa; between about 500 kDa and about 4,000 kDa; between about 500 kDa and about 3,500 kDa; between about 500 kDa and about 3,000 kDa; between about 500 kDa and about 2,500 kDa; between about 500 kDa and about 2,250 kDa; between about 500 kDa and about 2,000 kDa; between about 500 kDa and about 1,750 kDa; between about 500 kDa and about 1,500 kDa; between about 500 kDa and about 1,250 kDa; between about 500 kDa and about 1,000 kDa; between about 500 kDa and about 750 kDa; or between about 500 kDa and about 600 kDa.

Any number within any of the above ranges is contemplated as an embodiment of the disclosure.

In some embodiments, the purified polysaccharide is sized to a molecular weight of about 5 kDa, about 10 kDa, about 15 kDa, about 20 kDa, about 25 kDa, about 30 kDa, about 35 kDa, about 40 kDa, about 45 kDa, about 50 kDa, about 75 kDa, about 90 kDa, about 100 kDa, about 150 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1250 kDa, about 1500 kDa, about 1750 kDa, about 2000 kDa, about 2250 kDa, about 2500 kDa, about 2750 kDa, about 3000 kDa, about 3250 kDa, about 3500 kDa, about 3750 kDa or about 4,000 kDa.

In a preferred embodiment the purified polysaccharides, are capsular polysaccharide from serotypes 1, 3, 4, 5, 6A, 6B, 7F, 8, 9V, 10A, 11A, 12F, 14, 15A, 15B, 18C, 19A, 19F, 22F, 23F or 33F of S. pneumoniae, wherein the capsular polysaccharide has a molecular weight falling within one of the ranges or having about the size as described here above.

### 1.12 Sterile Filtration

In an embodiment, the purified solution of polysaccharide of the invention is sterilely filtered.

Therefore in an embodiment, the Ultrafiltration and/or Dialfiltration step of section 1.10 can optionally be followed by a sterile filtration step.

In an embodiment, the homogenizing/sizing step of section 1.11 if conducted can optionally be followed by a sterile filtration step.

In an embodiment, any of the step of sections 1.2 to 1.9 can optionally be followed by a sterile filtration step.

In an embodiment, sterile filtration is dead-end filtration (perpendicular filtration). In an embodiment, sterile filtration is tangential filtration.

In an embodiment, the solution is treated by a sterile filtration step wherein the filter has a nominal retention range of between about 0.01-0.2 micron, about 0.05-0.2 micron, about 0.1-0.2 micron or about 0.15-0.2 micron.

Any number within any of the above ranges is contemplated as an embodiment of the disclosure.

In an embodiment, the solution is treated by a sterile filtration step wherein the filter has a nominal retention range of about 0.05, about 0.1, about 0.15 or about 0.2 micron.

In an embodiment, the solution is treated by a sterile filtration step wherein the filter has a nominal retention range of about 0.2 micron.

In an embodiment, the solution is treated by a sterile filtration step wherein the filter has a filter capacity of about 25-1500 L/m², 50-1500 L/m², 75-1500 L/m², 100-1500 L/m², 150-1500 L/m², 200-1500 L/m², 250-1500 L/m², 300-1500 L/m², 350-1500 L/m², 400-1500 L/m², 500-1500 L/m², 750-1500 L/m², 1000-1500 L/m² or 1250-1500 L/m².

In an embodiment, the solution is treated by a sterile filtration step wherein the filter has a filter capacity of about 25-1000 L/m², 50-1000 L/m², 75-1000 L/m², 100-1000 L/m², 150-1000 L/m², 200-1000 L/m², 250-1000 L/m², 300-1000 L/m², 350-1000 L/m², 400-1000 L/m², 500-1000 L/m² or 750-1000 L/m².

In an embodiment, the solution is treated by a sterile filtration step wherein the filter has a filter capacity of 25-500 L/m², 50-500 L/m², 75-500 L/m², 100-500 L/m², 150-500 L/m², 200-500 L/m², 250-500 L/m², 300-500 L/m², 350-500 L/m² or 400-500 L/m².

In an embodiment, the solution is treated by a sterile filtration step wherein the filter has a filter capacity of 25-300 L/m², 50-300 L/m², 75-300 L/m², 100-300 L/m², 150-300 L/m², 200-300 L/m² or 250-300 L/m².

In an embodiment, the solution is treated by a sterile filtration step wherein the filter has a filter capacity of 25-250 L/m², 50-250 L/m², 75-250 L/m², 100-250 L/m² or 150-250 L/m², 200-250 L/m².

In an embodiment, the solution is treated by a sterile filtration step wherein the filter has a filter capacity of 25-100 L/m², 50-100 L/m² or 75-100 L/m².

Any number within any of the above ranges is contemplated as an embodiment of the disclosure.

In an embodiment, the solution is treated by a microfiltration step wherein the filter has a filter capacity of about 25, about 50, about 75, about 100, about 150, about 200, about 250, about 300, about 350, about 400, about 500, about 600, about 700, about 800, about 900, about 1000, about 1100, about 1200, about 1300, about 1400 or about 1500 L/m².

### 1.13 Final Material

The polysaccharide can be finally prepared as a liquid solution. The polysaccharide can be further processed (e.g. lyophilized as a dried powder, see WO2006/110381). Therefore in an embodiment, the polysaccharide is a dried powder.

In an embodiment, the polysaccharide is a freeze-dried cake.

### 2. Uses of the Purified Polysaccharides

The polysaccharide purified by the method of the present invention may be used as an antigen. Plain polysaccharides are used as antigens in vaccines (see the 23-valent unconjugated pneumococcal polysaccharide vaccine PNEUMOVAX).

The polysaccharide purified by the method of the present invention may also be conjugated to carrier protein(s) to obtain a glycoconjugate.

### 2.1. Glycoconjugates

The polysaccharide purified by the method of the present invention may be conjugated to carrier protein(s) to obtain a glycoconjugate.

For the purposes of the invention the term ‘glycoconjugate’ indicates a saccharide covalently linked to a carrier protein. In one embodiment a saccharide is linked directly to a carrier protein. In a second embodiment a saccharide is linked to a carrier protein through a spacer/linker.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

Purified polysaccharides by the method of the invention may be activated (e.g., chemically activated) to make them capable of reacting (e.g. with a linker or directly with the carrier protein) and then incorporated into glycoconjugates, as further described herein.

The purified polysaccharide maybe sized to a target molecular weight before conjugation e.g. by the methods disclosed at section 1.11 above. Therefore in an embodiment, the purified polysaccharide is sized before conjugation. In an embodiment, the purified polysaccharide as disclosed herein may be sized before conjugation to obtain an oligosaccharide. Oligosaccharides have a low number of repeat units (typically 5-15 repeat units) and are typically derived by sizing (e.g. hydrolysis) of the polysaccharide.

Preferably though, the saccharide to be used for conjugation is a polysaccharide. High molecular weight polysaccharides are able to induce certain antibody immune responses due to the epitopes present on the antigenic surface. The isolation and purification of high molecular weight polysaccharides is preferably contemplated for use in the conjugates of the present invention.

Therefore in an embodiment, the polysaccharide is sized and remains a polysaccharide. In an embodiment, the polysaccharide is not sized.

In some embodiments, the purified polysaccharide before conjugation (after sizing or unsized) has a molecular weight of between 5 kDa and 4,000 kDa. In other such embodiments, the purified polysaccharide has a molecular weight of between 10 kDa and 4,000 kDa. In other such embodiments, the purified polysaccharide has a molecular weight of between 50 kDa and 4,000 kDa. In further such embodiments, the polysaccharide has a molecular weight of between 50 kDa and 3,500 kDa; between 50 kDa and 3,000 kDa; between 50 kDa and 2,500 kDa; between 50 kDa and 2,000 kDa; between 50 kDa and 1,750 kDa; between 50 kDa and 1,500 kDa; between 50 kDa and 1,250 kDa; between 50 kDa and 1,000 kDa; between 50 kDa and 750 kDa; between 50 kDa and 500 kDa; between 100 kDa and 4,000 kDa; between 100 kDa and 3,500 kDa; 100 kDa and 3,000 kDa; 100 kDa and 2,500 kDa; 100 kDa and 2,250 kDa; between 100 kDa and 2,000 kDa; between 100 kDa and 1,750 kDa; between 100 kDa and 1,500 kDa; between 100 kDa and 1,250 kDa; between 100 kDa and 1,000 kDa; between 100 kDa and 750 kDa; between 100 kDa and 500 kDa; between 200 kDa and 4,000 kDa; between 200 kDa and 3,500 kDa; between 200 kDa and 3,000 kDa; between 200 kDa and 2,500 kDa; between 200 kDa and 2,250 kDa; between 200 kDa and 2,000 kDa; between 200 kDa and 1,750 kDa; between 200 kDa and 1,500 kDa; between 200 kDa and 1,250 kDa; between 200 kDa and 1,000 kDa; between 200 kDa and 750 kDa; or between 200 kDa and 500 kDa. In further such embodiments, the polysaccharide has a molecular weight of between 250 kDa and 3,500 kDa; between 250 kDa and 3,000 kDa; between 250 kDa and 2,500 kDa; between 250 kDa and 2,000 kDa; between 250 kDa and 1,750 kDa; between 250 kDa and 1,500 kDa; between 250 kDa and 1,250 kDa; between 250 kDa and 1,000 kDa; between 250 kDa and 750 kDa; between 250 kDa and 500 kDa; between 300 kDa and 4,000 kDa; between 300 kDa and 3,500 kDa; 300 kDa and 3,000 kDa; 300 kDa and 2,500 kDa; 300 kDa and 2,250 kDa; between 300 kDa and 2,000 kDa; between 300 kDa and 1,750 kDa; between 300 kDa and 1,500 kDa; between 300 kDa and 1,250 kDa; between 300 kDa and 1,000 kDa; between 300 kDa and 750 kDa; between 300 kDa and 500 kDa; between 500 kDa and 4,000 kDa; between 500 kDa and 3,500 kDa; between 500 kDa and 3,000 kDa; between 500 kDa and 2,500 kDa; between 500 kDa and 2,250 kDa; between 500 kDa and 2,000 kDa; between 500 kDa and 1,750 kDa; between 500 kDa and 1,500 kDa; between 500 kDa and 1,250 kDa; between 500 kDa and 1,000 kDa; between 500 kDa and 750 kDa; or between 500 kDa and 600 kDa.

Any number within any of the above ranges is contemplated as an embodiment of the disclosure.

In some embodiments, the purified polysaccharide has a molecular weight of about 5 kDa, 10 kDa, 15 kDa, 20 kDa, 25 kDa, 30 kDa, 35 kDa, 40 kDa, 45 kDa, 50 kDa, 75 kDa, 90 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, 1250 kDa, 1500 kDa, 1750 kDa, 2000 kDa, 2250 kDa, 2500 kDa, 2750 kDa, 3000 kDa, 3250 kDa, 3500 kDa, 3750 kDa or 4,000 kDa.

In an embodiment, the purified polysaccharide is a capsular saccharide (polysaccharide or oligosaccharide).

In an embodiment, the purified polysaccharide is a capsular polysaccharide from Escherichia coli. In an embodiment, the purified polysaccharide is a capsular polysaccharide from an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the purified polysaccharide is a capsular polysaccharide from an Uropathogenic Escherichia coli (UPEC).

In an embodiment, the purified polysaccharide is a capsular polysaccharide from an Escherichia coli serotype selected from the group consisting of serotypes O157:H7, O26:H11, O111:H- and O103:H2. In an embodiment, the purified polysaccharide is a capsular polysaccharide from an Escherichia coli serotype selected from the group consisting of serotypes O6:K2:H1 and O18:K1:H7. In an embodiment, the purified polysaccharide is a capsular polysaccharide from an Escherichia coli serotype selected from the group consisting of serotypes O45:K1, O17:K52:H18, O19:H34 and O7:K1. In an embodiment, the purified polysaccharide is a capsular polysaccharide from an Escherichia coli serotype O104:H4. In an embodiment, the purified polysaccharide is a capsular polysaccharide from Escherichia coli serotype O1:K12:H7. In an embodiment, the purified polysaccharide is a capsular polysaccharide from an Escherichia coli serotype O127:H6. In an embodiment, the purified polysaccharide is a capsular polysaccharide from an Escherichia coli serotype O139:H28. In an embodiment, the purified polysaccharide is a capsular polysaccharide from an Escherichia coli serotype O128:H2.

In a further embodiment, the purified polysaccharide is a capsular polysaccharide from Neisseria meningitidis. In an embodiment the purified polysaccharide is a capsular polysaccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC).

In another embodiment, the purified polysaccharide is a capsular polysaccharide from Klebsiella pneumoniae. In an embodiment the source of bacterial capsular polysaccharides is K. pneumoniae serogroup O1 (O1), K. pneumoniae serogroup O2 (O2), K. pneumoniae serogroup O2ac (O2ac), K. pneumoniae serogroup O3 (O3), K. pneumoniae serogroup O4 (O4), K. pneumoniae serogroup O5 (O5), K. pneumoniae serogroup O7 (O7), K. pneumoniae serogroup O8 (O8) or K. pneumoniae serogroup O9 (O9). In an embodiment the source of bacterial capsular polysaccharides is K. pneumoniae serogroup O1 (O1). In an embodiment the source of bacterial capsular polysaccharides is K. pneumoniae serogroup O2 (O2). In an embodiment the source of bacterial capsular polysaccharides is K. pneumoniae serogroup O2ac (O2ac). In an embodiment the source of bacterial capsular polysaccharides is K. pneumoniae serogroup O3 (O3). In an embodiment the source of bacterial capsular polysaccharides is K. pneumoniae serogroup O4 (O4). In an embodiment the source of bacterial capsular polysaccharides is K. pneumoniae serogroup O5 (O5). In an embodiment the source of bacterial capsular polysaccharides is K. pneumoniae serogroup O7 (O7). In an embodiment the source of bacterial capsular polysaccharides is K. pneumoniae serogroup O8 (O8). In an embodiment the source of bacterial capsular polysaccharides is K. pneumoniae serogroup O9 (O9).

Any suitable conjugation reaction can be used, with any suitable linker where necessary. See for example WO2007116028 pages 17-22.

The purified oligosaccharides or polysaccharides described herein are chemically activated to make the saccharides capable of reacting with the carrier protein.

In an embodiment, the glycoconjugate is prepared using reductive amination. Reductive amination involves two steps, (1) oxidation (activation) of the purified saccharide, (2) reduction of the activated saccharide and a carrier protein (e.g., CRM₁₉₇, DT, TT or PD) to form a glycoconjugate (see e.g. WO2015110941, WO2015110940).

As mentioned above, before oxidation, sizing of the polysaccharide to a target molecular weight (MW) range can be performed. Mechanical or chemical hydrolysis may be employed. Chemical hydrolysis may be conducted using acetic acid. In an embodiment, the size of the purified polysaccharide is reduced by mechanical homogenization.

In an embodiment, the purified polysacharide or oligosaccharide is conjugated to a carrier protein by a process comprising the step of:

(a) reacting said purified polysaccharide or oligosaccharide with an oxidizing agent;
(b) optionally quenching the oxidation reaction by addition of a quenching agent;
(c) compounding the activated polysaccharide or oligosaccharide of step (a) or (b) with a carrier protein; and
(d) reacting the compounded activated polysaccharide or oligosaccharide and carrier protein with a reducing agent to form a glycoconjugate.
Following the oxidation step (a) the saccharide is said to be activated and is referred to as “activated polysaccharide or oligosaccharide”.

The oxidation step (a) may involve reaction with periodate. For the purpose of the present invention, the term “periodate” includes both periodate and periodic acid; the term also includes both metaperiodate (IO₄⁻) and orthoperiodate (IO₆⁵⁻) and the various salts of periodate (e.g., sodium periodate and potassium periodate).

In a preferred embodiment, the oxidizing agent is sodium periodate. In an embodiment, the periodate used for the oxidation is metaperiodate. In an embodiment the periodate used for the oxidation is sodium metaperiodate.

The oxidation step (a) may involve reaction with a stable nitroxyl or nitroxide radical compound, such as piperidine-N-oxy or pyrrolidine-N-oxy compounds, in the presence of an oxidant to selectively oxidize primary hydroxyls of the said polysaccharide or oligosaccharide to produce an activated saccharide containing aldehyde groups (see WO2014097099). In an aspect, said stable nitroxyl or nitroxide radical compound is any one as disclosed at page 3 line 14 to page 4 line 7 of WO2014097099 and the oxidant is any one as disclosed at page 4 line 8 to 15 of WO2014097099. In an aspect, said stable nitroxyl or nitroxide radical compound is 2,2,6,6-tetramethyl-1-piperidinyloxy (TEMPO) and the oxidant is N-chlorosuccinimide (NCS).

In one embodiment, the quenching agent is as disclosed in WO2015110941 (see page 30 line 3 to 26).

In an embodiment, the reduction reaction (d) is carried out in aqueous solvent. In an embodiment, the reduction reaction (d) is carried out in aprotic solvent. In an embodiment, the reduction reaction (d) is carried out in DMSO (dimethylsulfoxide) or in DMF (dimethylformamide)) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In a preferred embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In an embodiment, the glycoconjugate is prepared using cyanylation chemistry. In an embodiment, the purified polysaccharide or oligosaccharide is activated with cyanogen bromide. The activation corresponds to cyanylation of the hydroxyl groups of the polysaccharide or oligosaccharide. The activated polysaccharide or oligosaccharide is then coupled directly or via a spacer (linker) group to an amino group on the carrier protein.

In an embodiment, the purified polysaccharide or oligosaccharide is activated with 1-cyano-4-dimethylamino pyridinium tetrafluoroborate (CDAP) to form a cyanate ester. The activated polysaccharide or oligosaccharide is then coupled directly or via a spacer (linker) group to an amino group on the carrier protein.

In an embodiment, the spacer could be cystamine or cysteamine to give a thiolated polysaccharide or oligosaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment, the glycoconjugate is prepared by using bis electrophilic reagents such as carbonyldiimidazole (CDI) or carbonylditriazole (CDT). In such an embodiment, the conjugation reaction is preferably made in aprotic solvents such as DMF or DMSO via a direct route or using bigeneric linkers (see e.g. WO2011041003).

In an embodiment, the glycoconjugate is prepared by a method of making glycoconjugates as disclosed in WO2014027302. The resulting glycoconjugate comprises a saccharide covalently conjugated to a carrier protein through a bivalent, heterobifunctional spacer (2-((2-oxoethyl)thio)ethyl)carbamate (eTEC). Alternatively, the glycoconjugate is prepared by a method of making glycoconjugates as disclosed in WO2015121783.

Other suitable conjugation techniques use carbodiimides (e.g. EDC (1-Ethyl-3-(3-dimethylaminopropyl) carbodiimide hydrochloride, EDC plus Sulfo NHS, CMC (1-Cyclohexyl-3-(2-morpholinoethyl)carbodiimide, DCC (N,N′-Dicyclohexyl carbodiimide), or DIC (diisopropyl carbodiimide).

In an embodiment, the polysaccharide or oligosaccaride is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is adipic acid dihydrazide (ADH). Other linkers include B-propionamido (WO 00/10599), nitrophenyl-ethylamine, haloalkyl halide), glycosidic linkages (U.S. Pat. Nos. 4,673,574, 4,808,700), hexane diamine and 6-aminocaproic acid (U.S. Pat. No. 4,459,286).

### Carrier Protein

A component of the glycoconjugate is a carrier protein to which the purified polysaccharide or oligosaccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugate is selected in the group consisting of: DT (Diphtheria toxin), TT (tetanus toxid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRMs, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa.

In a preferred embodiment, the carrier protein of the glycoconjugate is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile and PsaA.

In an embodiment, the carrier protein of the glycoconjugate is DT (Diphtheria toxoid). In another embodiment, the carrier protein of the glycoconjugate is TT (tetanus toxoid).

In another embodiment, the carrier protein of the glycoconjugate is PD (H. influenzae protein D; see, e.g., EP0594610 B).

In a preferred embodiment, the purified polysaccharide or oligosaccharide is conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the purified polysaccharide or oligosaccharide is conjugated to CRM₁₉₇ protein or the A chain of CRM₁₉₇ (see CN103495161). In an embodiment, the purified polysaccharide or oligosaccharide is conjugated the A chain of CRM₁₉₇ obtained via expression by genetically recombinant E. coli (see CN103495161).

Preferably the ratio of carrier protein to polysaccharide or oligosaccharide in the glycoconjugate is between 1:5 and 5:1; e.g. between 1:0.5 and 4:1, between 1:1 and 3.5:1, between 1.2:1 and 3:1, between 1.5:1 and 2.5:1; e.g. between 1:2 and 2.5:1 or between 1:1 and 2:1 (w/w). In an embodiment, the ratio of carrier protein to polysaccharide or oligosaccharide in the glycoconjugate is about 1:1, 1.1:1, 1.2:1, 1.3:1, 1.4:1, 1.5:1 or 1.6:1.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 2.2 Immunogenic Compositions

In an embodiment the invention relates to an immunogenic composition comprising any of the purified polysaccharides and/or glycoconjugates disclosed herein.

In an embodiment the invention relates to an immunogenic composition comprising any of the glycoconjugates disclosed herein.

In an embodiment the invention relates to an immunogenic composition comprising from 1 to 25 different glycoconjugates disclosed at section 2.1.

In an embodiment the invention relates to an immunogenic composition comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic compositions comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition.

In an embodiment the immunogenic composition is a 19-valent pneumococcal conjugate compositions. In an embodiment the immunogenic composition is a 20-valent pneumococcal conjugate composition.

In an embodiment said immunogenic composition comprises glycoconjugates from S. pneumoniae serotypes 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotypes 6A and 19A.

In an embodiment any of the immunogenic compositions above comprise in addition a glycoconjugate from S. pneumoniae serotype 3.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A, 12F and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In an embodiment the immunogenic composition of the invention comprises glycoconjugates from S. pneumoniae serotypes 8, 10A, 11A, 12F, 15B, 22F and 33F.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In a preferred embodiment though, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM197. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM197. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇. In an embodiment of any of the above immunogenic compositions, the glycoconjugate from S. pneumoniae serotype 3 is conjugated to CRM₁₉₇.

In an embodiment, the glycoconjugates of any of the above immunogenic compositions are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 20 different serotypes of S. pneumoniae.

In an embodiment the invention relates to an immunogenic composition comprising from 1 to 5 glycoconjugates from different N. meningitidis serogroups (1 to 5 meningococcal conjugates). In one embodiment the invention relates to an immunogenic composition comprising glycoconjugates from 1, 2, 3, 4, or 5 different N. meningitidis serogroups. In one embodiment the immunogenic compositions comprise 4 or 5 different N. meningitidis. In an embodiment the immunogenic composition is a 1, 2, 3, 4 or 5-valent meningococcal conjugate compositions. In an embodiment the immunogenic composition is a 2-valent meningococcal conjugate composition. In an embodiment the immunogenic composition is a 4-valent meningococcal conjugate composition. In an embodiment the immunogenic composition is a 5-valent meningococcal conjugate composition.

In an embodiment the immunogenic composition comprises a conjugated N. meningitidis serogroup Y capsular saccharide (MenY), and/or a conjugated N. meningitidis serogroup C capsular saccharide (MenC).

In an embodiment the immunogenic composition comprises a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic compositions comprise a conjugated N. meningitidis serogroup W135 capsular saccharide (MenW135), a conjugated N. meningitidis serogroup Y capsular saccharide (MenY), and/or a conjugated N. meningitidis serogroup C capsular saccharide (MenC).

In an embodiment the immunogenic composition comprises a conjugated N. meningitidis serogroup A capsular saccharide (MenA), a conjugated N. meningitidis serogroup W135 capsular saccharide (MenW135), a conjugated N. meningitidis serogroup Y capsular saccharide (MenY), a conjugated N. meningitidis serogroup C capsular saccharide (MenC) and/or a conjugated N. meningitidis serogroup X capsular saccharide (MenX).

In some embodiments, the immunogenic compositions disclosed herein may further comprise at least one, two or three adjuvants. In some embodiments, the immunogenic compositions disclosed herein may further comprise one adjuvant. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion orvortexed to generate a larger particle size emulsion, and (b) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, Mass.), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant.

The immunogenic compositions may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of polysaccahride or glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL, more preferably 0.2 mL to 1 mL, even more preferably a volume of about 0.5 mL.

2.3 Use as Antigens The polysaccharide purified by the method of the present invention and the conjugates disclosed herein may be used as antigens. For example, they may be part of a vaccine.

Therefore, in an embodiment, the polysaccharides purified by the method of the present invention or the glycoconjugates obtained using said polysaccharides are for use in generating an immune response in a subject. In one aspect, the subject is a mammal, such as a human, cat, sheep, pig, horse, bovine or dog. In one aspect, the subject is a human.

In an embodiment, the polysaccharides purified by the method of the present invention, the glycoconjugates obtained using said polysaccharides or the immunogenic compositions disclosed herein are for use in a vaccine.

In an embodiment, the polysaccharides purified by the method of the present invention, the glycoconjugates obtained using said polysaccharides or the immunogenic compositions disclosed herein are for use as a medicament.

The immunogenic compositions described herein may be used in various therapeutic or prophylactic methods for preventing, treating or ameliorating a bacterial infection, disease or condition in a subject. In particular, immunogenic compositions described herein may be used to prevent, treat or ameliorate a S. pneumoniae, S. aureus, E. faecalis, Haemophilus influenzae type b, E. coli, Neisseria meningitidis, S. agalactiae or Klebsiella pneumoniae infection, disease or condition in a subject.

Thus in one aspect, the disclosure provides a method of preventing, treating or ameliorating an infection, disease or condition associated with S. pneumoniae, S. aureus, E. faecalis, Haemophilus influenzae type b, E. coli, Neisseria meningitidis, S. agalactiae or Klebsiella pneumoniae in a subject, comprising administering to the subject an immunologically effective amount of an immunogenic composition of the disclosure (in particular an immunogenic composition comprising the corresponding polysaccharide or glycoconjugate thereof).

In an embodiment, the disclosure provides a method of inducing an immune response to S. pneumoniae, S. aureus, E. faecalis, Haemophilus influenzae type b, E. coli, Neisseria meningitidis, S. agalactiae or Klebsiella pneumoniae in a subject comprising administering to the subject an immunologically effective amount of an immunogenic composition of the disclosure (in particular an immunogenic composition comprising the corresponding polysaccharide or glycoconjugate thereof).

In an embodiment, the immunogenic compositions disclosed herein are for use as a vaccine. In such embodiments the immunogenic compositions described herein may be used to prevent S. pneumoniae, S. aureus, E. faecalis, Haemophilus influenzae type b, E. coli, Neisseria meningitidis or S. agalactiae infection in a subject. Thus in one aspect, the invention provides a method of preventing an infection by S. pneumoniae, S. aureus, E. faecalis, Haemophilus influenzae type b, E. coli, Neisseria meningitidis, S. agalactiae or Klebsiella pneumoniae in a subject comprising administering to the subject an immunologically effective amount of an immunogenic composition of the disclosure.

In one aspect, the subject is a mammal, such as a human, cat, sheep, pig, horse, bovine or dog. In one aspect, the subject is a human.

The immunogenic compositions of the present disclosure can be used to protect or treat a human susceptible to a S. pneumoniae, S. aureus, E. faecalis, Haemophilus influenzae type b, E. coli, Neisseria meningitidis, S. agalactiae or Klebsiella pneumoniae infection, by means of administering the immunogenic compositions via a systemic or mucosal route. In an embodiment, the immunogenic compositions disclosed herein are administered by intramuscular, intraperitoneal, intradermal or subcutaneous routes. In an embodiment, the immunogenic compositions disclosed herein are administered by intramuscular, intraperitoneal, intradermal or subcutaneous injection. In an embodiment, the immunogenic compositions disclosed herein are administered by intramuscular or subcutaneous injection.

In some cases, as little as one dose of the immunogenic composition according to the disclosure is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the disclosure is a single dose.

In an embodiment, the schedule of vaccination of the immunogenic composition according to the disclosure is a multiple dose schedule.

3. Saccharides Derived from E. coli

In one embodiment, the saccharide is produced in a recombinant Gram-negative bacterium. In one embodiment, the saccharide is produced in a recombinant E. coli cell. In one embodiment, the saccharide is produced in a recombinant Salmonella cell. Exemplary bacteria include E. coli O25K5H1, E. coli BD559, E. coli GAR2831, E. coli GAR865, E. coli GAR868, E. coli GAR869, E. coli GAR872, E. coli GAR878, E. coli GAR896, E. coli GAR1902, E. coli 025a ETC NR-5, E. coli O157:H7:K-, Salmonella enterica serovar Typhimurium strain LT2, E. coli GAR2401, Salmonella enterica serotype Enteritidis CVD 1943, Salmonella enterica serotype Typhimurium CVD 1925, Salmonella enterica serotype Paratyphi A CVD 1902, and Shigella flexneri CVD 1208S. In one embodiment, the bacterium is not E. coli GAR2401. This genetic approach towards saccharide production allows for efficient production of O-polysaccharides and O-antigen molecules as vaccine components.

The term “wzz protein,” as used herein, refers to a chain length determinant polypeptide, such as, for example, wzzB, wzz, wzz_{SF}, wZZ_{ST}, fepE, wzz_{fepE}, wzzl and wzz2. The GenBank accession numbers for the exemplary wzz gene sequences are AF011910 for E4991/76, AF011911 for F186, AF011912 for M70/1-1, AF011913 for 79/311, AF011914 for Bi7509- 41, AF011915 for C664-1992, AF011916 for C258-94, AF011917 for C722-89, and AF011919 for EDL933. The GenBank accession numbers for the G7 and Bi316-41 wzz genes sequences are U39305 and U39306, respectively. Further GenBank accession numbers for exemplary wzz gene sequences are NP_459581 for Salmonella enterica subsp. enterica serovar Typhimurium str. LT2 FepE; AIG66859 for E. coli O157:H7 Strain EDL933 FepE; NP_461024 for Salmonella enterica subsp. enterica serovar Typhimurium str. LT2 WzzB. NP_416531 for E. coli K-12 substr. MG1655 WzzB, NP_415119 for E. coli K-12 substr. MG1655 FepE. In preferred embodiments, the wzz family protein is any one of wzzB, wzz, wzz_{SF}, wzz_{ST}, fepE, wzz_{fepE}, wzz1 and wzz2, most preferably wzzB, more preferably fepE.

Exemplary wzzB sequences include:

>O25b 2401 WzzB
(SEQ ID NO: 20)
MRVENNNVSGQNHDPEQIDLIDLLVQLWRGKMTII
ISVIVAIALAIGYLAVAKEKWTSTAIITQPDVGQI
AGYNNAMNVIYGQAAPKVSDLQETLIGRFSSAFSA
LAETLDNQEEPEKLTIEPSVKNQQLPLTVSYVGQT
AEGAQMKLAQYIQQVDDKVNQELEKDLKDNIALGR
KNLQDSLRTQEVVAQEQKDLRIRQIQEALQYANQE
QVTKPQVQQTEDVTQDTLFLLGSEALESMIKHEAT
RPLVFSSNYYQTRQNLLDIESLKVDDLDIHAYRYV
MKPTLPIRRDSPKKAITLILAVLLGGMVGAGIVLG
RNALRNYNAK
>O25a:K5:H1 WzzB
(SEQ ID NO: 21)
MRVENNNVSGQNNDPEQIDLIDLLVQLWRGKMTII
ISVIVAIALAIGYLAVAKEKWTSTAIITQPDVGQI
AGYNNAMNVIYGQAAPKVSDLQETLIGRFSSAFSA
LAETLDNQDEPEKLTIEPSVKNQQLPLTVSYVGQT
AEGAQMKLAQYIQQVDDKVNQELEKDLKDNIALGR
KNLQDSLRTQEVVAQEQKDLRIRQIQEALQYANQA
QVTKPQIQQTGEDITQDTLFLLGSEALESMIKHEA
TRPLVFSPNYYQTRQNLLDIESLKVDDLDIHAYRY
VMKPTLPIRRDSPKKAITLILAVLLGGMVGAGIVL
GRNALRNYNAK
>O25a ETEC ATCC WzzB
(SEQ ID NO: 22)
MRVENNNVSGQNHDPEQIDLIDLLVQLWRGKMTII
ISVVVAIALAIGYLAVAKEKWTSTAIITQPDVGQI
AGYNNAMNVIYGQAAPKVSDLQETLIGRFSFAFSA
LAETLDNQKEPEKLTIEPSVKNQQLPLTVSYVGQT
AEDAQMKLAQYIQQVDDKVNQELEKDLKDNLALGR
KNLQDSLRTQEVVAQEQKDLRIRQIQEALQYANQA
QVTKPQIQQTGEDITQDTLFLLGSEALESMIKHEA
TRPLVFSPNYYQTRQNLLDIENLKVDDLDIHAYRY
VMKPTLPIRRDSPKKAITLILAVLLGGMVGAGIVL
GRNALRNYNSK
>K12 W3110 WzzB
(SEQ ID NO: 23)
MRVENNNVSGQNHDPEQIDLIDLLVQLWRGKMTII
ISVIVAIALAIGYLAVAKEKWTSTAIITQPDVGQI
AGYNNAMNVIYGQAAPKVSDLQETLIGRFSSAFSA
LAETLDNQEEREKLTIEPSVKNQQLPLTVSYVGQT
AEGAQMKLAQYIQQVDDKVNQELEKDLKDNIALGR
KNLQDSLRTQEVVAQEQKDLRIRQIQEALQYANQA
QVTKPQIQQTGEDITQDTLFLLGSEALESMIKHEA
TRPLVFSPNYYQTRQNLLDIESLKVDDLDIHAYRY
VMKPMLPIRRDSPKKAITLILAVLLGGMVGAGIVL
GRNALRNYNAK
>Salmonella LT2 WzzB
(SEQ ID NO: 24)
MTVDSNTSSGRGNDPEQIDLIELLLQLWRGKMTII
VAVIIAILLAVGYLMIAKEKWTSTAIITQPDAAQV
ATYTNALNVLYGGNAPKISEVQANFISRFSSAFSA
LSEVLDNQKEREKLTIEQSVKGQALPLSVSYVSTT
AEGAQRRLAEYIQQVDEEVAKELEVDLKDNITLQT
KTLQESLETQEVVAQEQKDLRIKQIEEALRYADEA
KITQPQIQQTQDVTQDTMFLLGSDALKSMIQNEAT
RPLVFSPAYYQTKQTLLDIKNLKVTADTVHVYRYV
MKPTLPVRRDSPKTAITLVLAVLLGGMIGAGIVLG
RNALRSYKPKAL

Exemplary FepE sequences include:

>O25b GAR2401 FepE
(SEQ ID NO: 15)
MSSLNIKQGSDAHFPDYPLASPSNNEIDLLNLISV
LWRAKKTVMAVVFAFACAGLLISFILPQKWTSAAV
VTPPEPVQWQELEKSFTKLRVLDLDIKIDRTEAFN
LFIKKFQSVSLLEEYLRSSPYVMDQLKEAKIDELD
LHRAIVALSEKMKAVDDNASKKKDEPSLYTSWTLS
FTAPTSEEAQTVLSGYIDYISTLVVKESLENVRNK
LEIKTQFEKEKLAQDRIKTKNQLDANIQRLNYSLD
IANAAGIKKPVYSNGQAVKDDPDFSISLGADGIER
KLEIEKAVTDVAELNGELRNRQYLVEQLTKAHVND
VNFTPFKYQLSPSLPVKKDGPGKAIIVILSALIGG
MVACGGVLLRYAMASRKQDAMMADHLV
>O25a:K5:H1 FepE
(SEQ ID NO: 16)
MSSLNIKQGSEAHFPEYPLASPSNNEIDLLNLIEV
LWRAKKTVMAVVFAFACAGLLISFILPQKWTSAAV
VTPPEPVQWQELEKTFTKLRVLDLDIKIDRTEAFN
LFIKKFQSVSLLEEYLRSSPYVMDQLKEAKIDPLD
LHRAIVALSEKMKAVDDNASKKKDESALYTSWTLS
FTAPTSEEAQKVLAGYIDYISALVVKESIENVRNK
LEIKTQFEKEKLAQDRIKTKNQLDANIQRLNYSLD
IANAAGIKKPVYSNGQAVKDDPDFSISLGADGIER
KLEIEKAVTDVAELNGELRNRQYLVEQLTKTNIND
VNFTPFKYQLRPSLPVKKDGQGKAIIVILSALVGG
MVACGGVLLRHAMASRKQDAMMADHLV
> O25a ETEC ATCC FepE
(SEQ ID NO: 17)
MSSLNIKQGSDAHFPDYPLASPSNNEIDLLNLISV
LWRAKKTVMAVVFAFACAGLLISFILPQKWTSAAV
VTPPEPVQWQELEKSFTKLRVLDLDIKIDRTEAFN
LFIKKFQSVSLLEEYLRSSPYVMDQLKEAKIDELD
LHRAIVALSEKMKAVDDNASKKKDEPSLYTSWTLS
FTAPTSEEAQTVLSGYIDYISTLVVKESLENVRNK
LEIKTQFEKEKLAQDRIKTKNQLDANIQRLNYSLD
IANAAGIKKPVYSNGQAVKDDPDFSISLGADGIER
KLEIEKAVTDVAELNGELRNRQYLVEQLTKAHVND
VNFTPFKYQLSPSLPVKKDGPGKAIIVILSALIGG
MVACGGVLLRYAMASRKQDAMMADHLV
> O157 FepE
(SEQ ID NO: 18)
MSSLNIKQGSDAHFPDYPLASPSNNEIDLLNLISV
LWRAKKTVMAVVFAFACAGLLISFILPQKWTSAAV
VTPPEPVQWQELEKTFTKLRVLDLDIKIDRTEAFN
LFIKKFQSVSLLEEYLRSSPYVMDQLKEAKIDELD
LHRAIVALSEKMKAVDDNASKKKDEPSLYTSWTLS
FTAPTSEEAQTVLSGYIDYISALVVKESIENVRNK
LEIKTQFEKEKLAQDRIKMKNQLDANIQRLNYSLD
IANAAGIKKPVYSNGQAVKDDPDFSISLGADGIER
KLEIEKAVTDVAELNGELRNRQYLVEQLTKANIND
VNFTPFKYQLSPSLPVKKDGPGKAIIVILSALIGG
MVACGSVLLRYAMASRKQDAMMADHLV
>Salmonella LT2 FepE
(SEQ ID NO: 19)
MPSLNVKQEKNQSFAGYSLPPANSHEIDLFSLIEV
LWQAKRRILATVFAFACVGLLLSFLLPQKWTSQAI
VTPAESVQWQGLERTLTALRVLDMEVSVDRGSVFN
LFIKKFSSPSLLEEYLRSSPYVMDQLKGAQIDEQD
LHRAIVLLSEKMKAVDSNVGKKNETSLFTSWTLSF
TAPTREEAQKVLAGYIQYISDIVVKETLENIRNQL
EIKTRYEQEKLAMDRVRLKNQLDANIQRLHYSLEI
ANAAGIKRPVYSNGQAVKDDPDFSISLGADGISRK
LEIEKGVTDVAEIDGDLRNRQYHVEQLAAMNVSDV
KFTPFKYQLSPSLPVKKDGPGKAIIIILAALIGGM
MACGGVLLRHAMVSRKMENALAIDERLV

In some embodiments, a modified saccharide (modified as compared to the corresponding wild-type saccharide) may be produced by expressing (not necessarily overexpressing) a wzz family protein (e.g., fepE) from a Gram-negative bacterium in a Gram-negative bacterium and/or by switching off (i.e., repressing, deleting, removing) a second wzz gene (e.g., wzzB) to generate high molecular weight saccharides, such as lipopolysaccharides, containing intermediate or long O-antigen chains. For example, the modified saccharides may be produced by expressing (not necessarily overexpressing) wzz2 and switching off wzzl. Or, in the alternative, the modified saccharides may be produced by expressing (not necessarily overexpressing) wzzfepE and switching off wzzB. In another embodiment, the modified saccharides may be produced by expressing (not necessarily overexpressing) wzzB but switching off wzzfepE. In another embodiment, the modified saccharides may be produced by expressing fepE. Preferably, the wzz family protein is derived from a strain that is heterologous to the host cell.

In one aspect, the invention relates to saccharides produced by expressing a wzz family protein, preferably fepE, in a Gram-negative bacterium to generate high molecular weight saccharides containing intermediate or long O-antigen chains, which have an increase of at least 1, 2, 3, 4, or 5 repeating units, as compared to the corresponding wild-type O-polysaccharide. In one aspect, the invention relates to saccharides produced by a Gram-negative bacterium in culture that expresses (not necessarily overexpresses) a wzz family protein (e.g., wzzB) from a Gram-negative bacterium to generate high molecular weight saccharides containing intermediate or long O-antigen chains, which have an increase of at least 1, 2, 3, 4, or 5 repeating units, as compared to the corresponding wild-type O-antigen. See description of O-polysaccharides and O-antigens below for additional exemplary saccharides having increased number of repeat units, as compared to the corresponding wild-type saccharides. A desired chain length is the one which produces improved or maximal immunogenicity in the context of a given vaccine construct.

In another embodiment, the saccharide includes any one Formula selected from Table 1, wherein the number of repeat units n in the saccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide. Methods of determining the length of saccharides are known in the art. Such methods include nuclear magnetic resonance, mass spectroscopy, and size exclusion chromatography.

In a preferred embodiment, the invention relates to a saccharide produced in a recombinant E. coli host cell, wherein the gene for an endogenous wzz O-antigen length regulator (e.g., wzzB) is deleted and is replaced by a (second) wzz gene from a Gram-negative bacterium heterologous to the recombinant E. coli host cell (e.g., Salmonella fepE) to generate high molecular weight saccharides, such as lipopolysaccharides, containing intermediate or long O-antigen chains. In some embodiments, the recombinant E. coli host cell includes a wzz gene from Salmonella, preferably from Salmonella enterica.

In one embodiment, the host cell includes the heterologous gene for a wzz family protein as a stably maintained plasmid vector. In another embodiment, the host cell includes the heterologous gene for a wzz family protein as an integrated gene in the chromosomal DNA of the host cell. Methods of stably expressing a plasmid vector in an E. coli host cell and methods of integrating a heterologous gene into the chromosome of an E. coli host cell are known in the art. In one embodiment, the host cell includes the heterologous genes for an O-antigen as a stably maintained plasmid vector. In another embodiment, the host cell includes the heterologous genes for an O-antigen as an integrated gene in the chromosomal DNA of the host cell. Methods of stably expressing a plasmid vector in an E. coli host cell and a Salmonella host cell are known in the art. Methods of integrating a heterologous gene into the chromosome of an E. coli host cell and a Salmonella host cell are known in the art.

In one aspect, the recombinant host cell is cultured in a medium that comprises a carbon source. Carbon sources for culturing E. coli are known in the art. Exemplary carbon sources include sugar alcohols, polyols, aldol sugars or keto sugars including but not limited to arabinose, cellobiose, fructose, glucose, glycerol, inositol, lactose, maltose, mannitol, mannose, rhamnose, raffinose, sorbitol, sorbose, sucrose, trehalose, pyruvate, succinate and methylamine. In a preferred embodiment, the medium includes glucose. In some embodiments, the medium includes a polyol or aldol sugar, for example, mannitol, inositol, sorbose, glycerol, sorbitol, lactose and arabinose as the carbon source. All of the carbon sources may be added to the medium before the start of culturing, or it may be added step by step or continuously during culturing.

An exemplary culture medium for the recombinant host cell includes an element selected from any one of KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O. Preferably, the medium includes KH₂PO₄, K₂HPO₄, (NH₄)₂SO₄, sodium citrate, Na₂SO₄, aspartic acid, glucose, MgSO₄, FeSO₄-7H₂O, Na₂MoO₄-2H₂O, H₃BO₃, CoCl₂-6H₂O, CuCl₂-2H₂O, MnCl₂-4H₂O, ZnCl₂ and CaCl₂-2H₂O.

The medium used herein may be solid or liquid, synthetic (i.e. man-made) or natural, and may include sufficient nutrients for the cultivation of the recombinant host cell. Preferably, the medium is a liquid medium.

In some embodiments, the medium may further include suitable inorganic salts. In some embodiments, the medium may further include trace nutrients. In some embodiments, the medium may further include growth factors. In some embodiments, the medium may further include an additional carbon source. In some embodiments, the medium may further include suitable inorganic salts, trace nutrients, growth factors, and a supplementary carbon source.

Inorganic salts, trace nutrients, growth factors, and supplementary carbon sources suitable for culturing E. coli are known in the art.

In some embodiments, the medium may include additional components as appropriate, such as peptone, N-Z Amine, enzymatic soy hydrosylate, additional yeast extract, malt extract, supplemental carbon sources and various vitamins. In some embodiments, the medium does not include such additional components, such as peptone, N-Z Amine, enzymatic soy hydrosylate, additional yeast extract, malt extract, supplemental carbon sources and various vitamins.

Illustrative examples of suitable supplemental carbon sources include, but are not limited to other carbohydrates, such as glucose, fructose, mannitol, starch or starch hydrolysate, cellulose hydrolysate and molasses; organic acids, such as acetic acid, propionic acid, lactic acid, formic acid, malic acid, citric acid, and fumaric acid; and alcohols, such as glycerol, inositol, mannitol and sorbitol.

In some embodiments, the medium further includes a nitrogen source. Nitrogen sources suitable for culturing E. coli are known in the art. Illustrative examples of suitable nitrogen sources include, but are not limited to ammonia, including ammonia gas and aqueous ammonia; ammonium salts of inorganic or organic acids, such as ammonium chloride, ammonium nitrate, ammonium phosphate, ammonium sulfate and ammonium acetate; urea; nitrate or nitrite salts, and other nitrogen-containing materials, including amino acids as either pure or crude preparations, meat extract, peptone, fish meal, fish hydrolysate, corn steep liquor, casein hydrolysate, soybean cake hydrolysate, yeast extract, dried yeast, ethanol-yeast distillate, soybean flour, cottonseed meal, and the like.

In some embodiments, the medium includes an inorganic salt. Illustrative examples of suitable inorganic salts include, but are not limited to salts of potassium, calcium, sodium, magnesium, manganese, iron, cobalt, zinc, copper, molybdenum, tungsten and other trace elements, and phosphoric acid.

In some embodiments, the medium includes appropriate growth factors. Illustrative examples of appropriate trace nutrients, growth factors, and the like include, but are not limited to coenzyme A, pantothenic acid, pyridoxine-HCl, biotin, thiamine, riboflavin, flavine mononucleotide, flavine adenine dinucleotide, DL-6, 8-thioctic acid, folic acid, Vitamin B₁₂, other vitamins, amino acids such as cysteine and hydroxyproline, bases such as adenine, uracil, guanine, thymine and cytosine, sodium thiosulfate, p- or r-aminobenzoic acid, niacinamide, nitriloacetate, and the like, either as pure or partially purified chemical compounds or as present in natural materials. The amounts may be determined empirically by one skilled in the art according to methods and techniques known in the art.

In another embodiment, the modified saccharide (as compared to the corresponding wild-type saccharide) described herein is synthetically produced, for example, in vitro. Synthetic production or synthesis of the saccharides may facilitate the avoidance of cost- and time-intensive production processes. In one embodiment, the saccharide is synthetically synthesized, such as, for example, by using sequential glycosylation strategy or a combination of sequential glycosylations and [3+2] block synthetic strategy from suitably protected monosaccharide intermediates. For example, thioglycosides and glycosyl trichloroacetimidate derivatives may be used as glycosyl donors in the glycosylations. In one embodiment, a saccharide that is synthetically synthesized in vitro has the identical structure to a saccharide produced by recombinant means, such as by manipulation of a wzz family protein described above.

The saccharide produced (by recombinant or synthetic means) includes a structure derived from any E. coli serotype including, for example, any one of the following E. coli serotypes: O1 (e.g., O1A, O1B, and O1C), O2, O3, O4 (e.g., O4:K52 and O4:K6), O5 (e.g., O5ab and O5ac (strain 180/C3)), O6 (e.g., O6:K2; K13; K15 and O6:K54), O7, O8, O9, O10, O11, O12, O13, O14, O15, O16, O17, O18 (e.g., O18A, O18ac, O18A1, O18B, and O18B1), O19, O20, O21, O22, O23 (e.g., O23A), O24, O25 (e.g., O25a and O25b), O26, O27, O28, O29, O30, O32, O33, O34, O35, O36, O37, O38, O39, O40, O41, O42, O43, O44, O45 (e.g., O45 and O45rel), O46, O48, O49, O50, O51, O52, O53, O54, O55, O56, O57, O58, O59, O60, O61, O62, 62D1, O63, O64, O65, O66, O68, O69, O70, O71, O73 (e.g., O73 (strain 73-1)), O74, O75, O76, O77, O78, O79, O80, O81, O82, O83, O84, O85, O86, O87, O88, O89, O90, O91, O92, O93, O95, O96, O97, O98, O99, O100, O101, O102, O103, O104, O105, O106, O107, O108, O109, O110, O111, O112, O113, O114, O115, O116, O117, O118, O119, O120, O121, O123, O124, O125, O126, O127, O128, O129, O130, O131, O132, O133, O134, O135, O136, O137, O138, O139, O140, O141, O142, O143, O144, O145, O146, O147, O148, O149, O150, O151, O152, O153, O154, O155, O156, O157, O158, O159, O160, O161, O162, O163, O164, O165, O166, O167, O168, O169, O170, O171, O172, O173, O174, O175, O176, O177, O178, O179, O180, O181, O182, O183, O184, O185, O186, and O187.

The individual polysaccharides are typically purified (enriched with respect to the amount of polysaccharide-protein conjugate) through methods known in the art, such as, for example, dialysis, concentration operations, diafiltration operations, tangential flow filtration, precipitation, elution, centrifugation, precipitation, ultra-filtration, depth filtration, and/or column chromatography (ion exchange chromatography, multimodal ion exchange chromatography, DEAE, and hydrophobic interaction chromatography). Preferably, the polysaccharides are purified through a method that includes tangential flow filtration.

Purified polysaccharides may be activated (e.g., chemically activated) to make them capable of reacting (e.g., either directly to the carrier protein or via a linker such as an eTEC spacer) and then incorporated into glycoconjugates of the invention, as further described herein.

In one preferred embodiment, the saccharide of the invention is derived from an E. coli serotype, wherein the serotype is O25a. In another preferred embodiment, the serotype is O25b. In another preferred embodiment, the serotype is O1A. In another preferred embodiment, the serotype is O2. In another preferred embodiment, the serotype is O6. In another preferred embodiment, the serotype is O17. In another preferred embodiment, the serotype is O15. In another preferred embodiment, the serotype is O18A. In another preferred embodiment, the serotype is O75. In another preferred embodiment, the serotype is O4. In another preferred embodiment, the serotype is O16. In another preferred embodiment, the serotype is O13. In another preferred embodiment, the serotype is O7. In another preferred embodiment, the serotype is O8. In another preferred embodiment, the serotype is O9.

As used herein, reference to any of the serotypes listed above, refers to a serotype that encompasses a repeating unit structure (O-unit, as described below) known in the art and is unique to the corresponding serotype. For example, the term “O25a” serotype (also known in the art as serotype “O25”) refers to a serotype that encompasses Formula O25 shown in Table 1. As another example, the term “O25b” serotype refers to a serotype that encompasses Formula O25b shown in Table 1.

As used herein, the serotypes are referred generically herein unless specified otherwise such that, for example, the term Formula “O18” refers generically to encompass Formula O18A, Formula O18ac, Formula 18A1, Formula O18B, and Formula O18B1.

As used herein, the term “O1” refers generically to encompass the species of Formula that include the generic term “O1” in the Formula name according to Table 1, such as any one of Formula O1A, Formula O1A1, Formula O1B, and Formula O1C, each of which is shown in Table 1. Accordingly, an “O1 serotype” refers generically to a serotype that encompasses any one of Formula O1A, Formula O1A1, Formula O1B, and Formula O1C.

As used herein, the term “O6” refers generically to species of Formula that include the generic term “O6” in the Formula name according to Table 1, such as any one of Formula O6:K2; K13; K15; and O6:K54, each of which is shown in Table 1. Accordingly, an “O6 serotype” refers generically to a serotype that encompasses any one of Formula O6:K2; K13; K15; and O6:K54.

Other examples of terms that refer generically to species of a Formula that include the generic term in the Formula name according to Table 1 include: “O4”, “O5”, “O18”, and “O45”.

As used herein, the term “O2” refers to Formula O2 shown in Table 1. The term “O2 O-antigen” refers to a saccharide that encompasses Formula O2 shown in Table 1.

As used herein, reference to an O-antigen from a serotype listed above refers to a saccharide that encompasses the formula labeled with the corresponding serotype name. For example, the term “O25B O-antigen” refers to a saccharide that encompasses Formula O25B shown in Table 1.

As another example, the term “O1 O-antigen” generically refers to a saccharide that encompasses a Formula including the term “O1,” such as the Formula O1A, Formula O1A1, Formula O1B, and Formula O1C, each of which are shown in Table 1.

As another example, the term “O6 O-antigen” generically refers to a saccharide that encompasses a Formula including the term “O6,” such as Formula O6:K2; Formula O6:K13; Formula O6:K15 and Formula O6:K54, each of which are shown in Table 1.

### O-Polysaccharide

As used herein, the term “O-polysaccharide” refers to any structure that includes an O-antigen, provided that the structure does not include a whole cell or Lipid A. For example, in one embodiment, the O-polysaccharide includes a lipopolysaccharide wherein the Lipid A is not bound. The step of removing Lipid A is known in the art and includes, as an example, heat treatment with addition of an acid. An exemplary process includes treatment with 1% acetic acid at 100° C. for 90 minutes. This process is combined with a process of isolating Lipid A as removed. An exemplary process for isolating Lipid A includes ultracentrifugation.

In one embodiment, the O-polysaccharide refers to a structure that consists of the O-antigen, in which case, the O-polysaccharide is synonymous with the term O-antigen. In one preferred embodiment, the O-polysaccharide refers to a structure that includes repeating units of the O-antigen, without the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety. In another preferred embodiment, the O-polysaccharide refers to a structure that includes an O-antigen and a core saccharide. In another embodiment, the O-polysaccharide refers to a structure that includes an O-antigen, a core saccharide, and a KDO moiety.

Methods of purifying an O-polysaccharide, which includes the core oligosaccharide, from LPS are known in the art. For example, after purification of LPS, purified LPS may be hydrolyzed by heating in 1% (v/v) acetic acid for 90 minutes at 100 degrees Celsius, followed by ultracentrifugation at 142,000×g for 5 hours at 4 degrees Celsius. The supernatant containing the O-polysaccharide is freeze-dried and stored at 4 degrees Celsius. In certain embodiments, deletion of capsule synthesis genes to enable simple purification of O-polysaccharide is described.

The O-polysaccharide can be isolated by methods including, but not limited to mild acid hydrolysis to remove lipid A from LPS. Other embodiments may include use of hydrazine as an agent for O-polysaccharide preparation. Preparation of LPS can be accomplished by known methods in the art.

In certain embodiments, the O-polysaccharides purified from wild-type, modified, or attenuated Gram-negative bacterial strains that express (not necessarily overexpress) a Wzz protein (e.g., wzzB) are provided for use in conjugate vaccines. In preferred embodiments, the O-polysaccharide chain is purified from the Gram-negative bacterial strain expressing (not necessarily overexpressing) wzz protein for use as a vaccine antigen either as a conjugate or complexed vaccine.

In one embodiment, the O-polysaccharide has a molecular weight that is increased by about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, 50-fold, 51-fold, 52-fold, 53-fold, 54-fold, 55-fold, 56-fold, 57-fold, 58-fold, 59-fold, 60-fold, 61-fold, 62-fold, 63-fold, 64-fold, 65-fold, 66-fold, 67-fold, 68-fold, 69-fold, 70-fold, 71-fold, 72-fold, 73-fold, 74-fold, 75-fold, 76-fold, 77-fold, 78-fold, 79-fold, 80-fold, 81-fold, 82-fold, 83-fold, 84-fold, 85-fold, 86-fold, 87-fold, 88-fold, 89-fold, 90-fold, 91-fold, 92-fold, 93-fold, 94-fold, 95-fold, 96-fold, 97-fold, 98-fold, 99-fold, 100-fold or more, as compared to the corresponding wild-type O-polysaccharide. In a preferred embodiment, the O-polysaccharide has a molecular weight that is increased by at least 1-fold and at most 5-fold, as compared to the corresponding wild-type O-polysaccharide. In another embodiment, the O-polysaccharide has a molecular weight that is increased by at least 2-fold and at most 4-fold, as compared to the corresponding wild-type O-polysaccharide. An increase in molecular weight of the O-polysaccharide, as compared to the corresponding wild-type O-polysaccharide, is preferably associated with an increase in number of O-antigen repeat units. In one embodiment, the increase in molecular weight of the O-polysaccharide is due to the wzz family protein.

In one embodiment, the O-polysaccharide has a molecular weight that is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 kDa or more, as compared to the corresponding wild-type O-polysaccharide. In one embodiment, the O-polysaccharide of the invention has a molecular weight that is increased by at least 1 and at most 200 kDa, as compared to the corresponding wild-type O-polysaccharide. In one embodiment, the molecular weight is increased by at least 5 and at most 200 kDa. In one embodiment, the molecular weight is increased by at least 10 and at most 200 kDa. In one embodiment, the molecular weight is increased by at least 12 and at most 200 kDa. In one embodiment, the molecular weight is increased by at least 15 and at most 200 kDa. In one embodiment, the molecular weight is increased by at least 18 and at most 200 kDa. In one embodiment, the molecular weight is increased by at least 20 and at most 200 kDa. In one embodiment, the molecular weight is increased by at least 21 and at most 200 kDa. In one embodiment, the molecular weight is increased by at least 22 and at most 200 kDa. In one embodiment, the molecular weight is increased by at least 30 and at most 200 kDa. In one embodiment, the molecular weight is increased by at least 1 and at most 100 kDa. In one embodiment, the molecular weight is increased by at least 5 and at most 100 kDa. In one embodiment, the molecular weight is increased by at least 10 and at most 100 kDa. In one embodiment, the molecular weight is increased by at least 12 and at most 100 kDa. In one embodiment, the molecular weight is increased by at least 15 and at most 100 kDa. In one embodiment, the molecular weight is increased by at least 20 and at most 100 kDa. In one embodiment, the molecular weight is increased by at least 1 and at most 75 kDa. In one embodiment, the molecular weight is increased by at least 5 and at most 75 kDa. In one embodiment, the molecular weight is increased by at least 10 and at most 75 kDa. In one embodiment, the molecular weight is increased by at least 12 and at most 75 kDa. In one embodiment, the molecular weight is increased by at least 15 and at most 75 kDa. In one embodiment, the molecular weight is increased by at least 18 and at most 75 kDa. In one embodiment, the molecular weight is increased by at least 20 and at most 75 kDa. In one embodiment, the molecular weight is increased by at least 30 and at most 75 kDa. In one embodiment, the molecular weight is increased by at least 10 and at most 90 kDa. In one embodiment, the molecular weight is increased by at least 12 and at most 85 kDa. In one embodiment, the molecular weight is increased by at least 10 and at most 75 kDa. In one embodiment, the molecular weight is increased by at least 10 and at most 70 kDa. In one embodiment, the molecular weight is increased by at least 10 and at most 60 kDa. In one embodiment, the molecular weight is increased by at least 10 and at most 50 kDa. In one embodiment, the molecular weight is increased by at least 10 and at most 49 kDa. In one embodiment, the molecular weight is increased by at least 10 and at most 48 kDa. In one embodiment, the molecular weight is increased by at least 10 and at most 47 kDa. In one embodiment, the molecular weight is increased by at least 10 and at most 46 kDa. In one embodiment, the molecular weight is increased by at least 20 and at most 45 kDa. In one embodiment, the molecular weight is increased by at least 20 and at most 44 kDa. In one embodiment, the molecular weight is increased by at least 20 and at most 43 kDa. In one embodiment, the molecular weight is increased by at least 20 and at most 42 kDa. In one embodiment, the molecular weight is increased by at least 20 and at most 41 kDa. Such an increase in molecular weight of the O-polysaccharide, as compared to the corresponding wild-type O-polysaccharide, is preferably associated with an increase in number of O-antigen repeat units. In one embodiment, the increase in molecular weight of the O-polysaccharide is due to the wzz family protein.

In another embodiment, the O-polysaccharide includes any one Formula selected from Table 1, wherein the number of repeat units n in the O-polysaccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide.

### O-Antigen

The O-antigen is part of the lipopolysaccharide (LPS) in the outer membrane of Gram-negative bacteria. The O-antigen is on the cell surface and is a variable cell constituent. The variability of the O-antigen provides a basis for serotyping of Gram-negative bacteria. The current E. coli serotyping scheme includes O-polysaccharides 1 to 181.

The O-antigen includes oligosaccharide repeating units (O-units), the wild type structure of which usually contains two to eight residues from a broad range of sugars. The O-units of exemplary E. coli O-antigens are shown in Table 1. The O-units of exemplary K. pneumoniae O-antigens are shown in Table 1a.

In one embodiment, saccharide of the invention may be one oligosaccharide unit. In one embodiment, saccharide of the invention is one repeating oligosaccharide unit of the relevant serotype. In such embodiments, the saccharide may include a structure selected from any one of Formula O8, Formula O9a, Formula O9, Formula O20ab, Formula O20ac, Formula O52, Formula O97, and Formula O101.

In one embodiment, saccharide of the invention may be oligosaccharides. Oligosaccharides have a low number of repeat units (typically 5-15 repeat units) and are typically derived synthetically or by hydrolysis of polysaccharides. In such embodiments, the saccharide may include a structure selected from any one of Formula O8, Formula O9a, Formula O9, Formula O20ab, Formula O20ac, Formula O52, Formula O97, and Formula O101.

Preferably, all of the saccharides of the present invention and in the immunogenic compositions of the present invention are polysaccharides. High molecular weight polysaccharides may induce certain antibody immune responses due to the epitopes present on the antigenic surface. The isolation and purification of high molecular weight polysaccharides are preferably contemplated for use in the conjugates, compositions and methods of the present invention.

In some embodiments, the number of repeat 0 units in each individual O-antigen polymer (and therefore the length and molecular weight of the polymer chain) depends on the wzz chain length regulator, an inner membrane protein. Different wzz proteins confer different ranges of modal lengths (4 to >100 repeat units). The term “modal length” refers to the number of repeating O-units. Gram-negative bacteria often have two different Wzz proteins that confer two distinct OAg modal chain lengths, one longer and one shorter. The expression (not necessarily the overexpression) of wzz family proteins (e.g., wzzB) in Gram-negative bacteria may allow for the manipulation of O-antigen length, to shift or to bias bacterial production of O-antigens of certain length ranges, and to enhance production of high-yield large molecular weight lipopolysaccharides. In one embodiment, a “short” modal length as used herein refers to a low number of repeat O-units, e.g., 1-20. In one embodiment, a “long” modal length as used herein refers to a number of repeat O-units greater than 20 and up to a maximum of 40. In one embodiment, a “very long” modal length as used herein refers to greater than 40 repeat O-units.

In one embodiment, the saccharide produced has an increase of at least 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, or 100 repeating units, as compared to the corresponding wild-type O-polysaccharide.

In another embodiment, the saccharide of the invention has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide. Preferably, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide.

Methods of determining the number of repeat units in the saccharide are also known in the art. For example, the number of repeat units (or “n” in the Formula) may be calculated by dividing the molecular weight of the polysaccharide (without the molecular weight of the core saccharide or KDO residue) by the molecular weight of the repeat unit (i.e., molecular weight of the structure in the corresponding Formula, shown for example in Table 1, which may be theoretically calculated as the sum of the molecular weight of each monosaccharide within the Formula). The molecular weight of each monosaccharide within the Formula is known in the art.

The molecular weight of a repeat unit of Formula O25b, for example, is about 862 Da. The molecular weight of a repeat unit of Formula O1a, for example, is about 845 Da. The molecular weight of a repeat unit of Formula O2, for example, is about 829 Da. The molecular weight of a repeat unit of Formula O6, for example, is about 893 Da. When determining the number of repeat units in a conjugate, the carrier protein molecular weight and the protein:polysaccharide ratio is factored into the calculation. As defined herein, “n” refers to the number of repeating units (represented in brackets in Table 1) in a polysaccharide molecule. As is known in the art, in biological macromolecules, repeating structures may be interspersed with regions of imperfect repeats, such as, for example, missing branches. In addition, it is known in the art that polysaccharides isolated and purified from natural sources such as bacteria may be heterogenous in size and in branching. In such a case, n may represent an average or median value for n for the molecules in a population.

In one embodiment, the O-polysaccharide has an increase of at least one repeat unit of an O-antigen, as compared to the corresponding wild-type O-polysaccharide. The repeat units of O-antigens are shown in Table 1 and Table 1a. In one embodiment, the O-polysaccharide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more total repeat units. Preferably, the saccharide has a total of at least 3 to at most 80 repeat units. In another embodiment, the O-polysaccharide has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide. In one embodiment, the saccharide includes an O-antigen wherein n in any of the O-antigen formulas (such as, for example, the Formulas shown in Table 1) is an integer of at least 1, 2, 3, 4, 5, 10, 20, 21, 22, 23, 24, 25, 26, 27, 28, 29, 30, 31, 32, 33, 34, 35, 36, 37, 38, 39, 40, and at most 200, 100, 99, 98, 97, 96, 95, 94, 93, 92, 91, 90, 89, 88, 87, 86, 81, 80, 79, 78, 77, 76, 75, 74, 73, 72, 71, 70, 69, 68, 67, 66, 65, 60, 59, 58, 57, 56, 55, 54, 53, 52, 51, or 50. Any minimum value and any maximum value may be combined to define a range. Exemplary ranges include, for example, at least 1 to at most 1000; at least 10 to at most 500; and at least 20 to at most 80, preferably at most 90. In one preferred embodiment, n is at least 31 to at most 90. In a preferred embodiment, n is 40 to 90, more preferably 60 to 85.

In one embodiment, the saccharide includes an O-antigen wherein n in any one of the O-antigen Formulas is at least 1 and at most 200. In one embodiment, n in any one of the O-antigen Formulas is at least 5 and at most 200. In one embodiment, n in any one of the O-antigen Formulas is at least 10 and at most 200. In one embodiment, n in any one of the O-antigen Formulas is at least 25 and at most 200. In one embodiment, n in any one of the O-antigen Formulas is at least 50 and at most 200. In one embodiment, n in any one of the O-antigen Formulas is at least 75 and at most 200. In one embodiment, n in any one of the O-antigen Formulas is at least 100 and at most 200. In one embodiment, n in any one of the O-antigen Formulas is at least 125 and at most 200. In one embodiment, n in any one of the O-antigen Formulas is at least 150 and at most 200. In one embodiment, n in any one of the O-antigen Formulas is at least 175 and at most 200. In one embodiment, n in any one of the O-antigen Formulas is at least 1 and at most 100. In one embodiment, n in any one of the O-antigen Formulas is at least 5 and at most 100. In one embodiment, n in any one of the O-antigen Formulas is at least 10 and at most 100. In one embodiment, n in any one of the O-antigen Formulas is at least 25 and at most 100. In one embodiment, n in any one of the O-antigen Formulas is at least 50 and at most 100. In one embodiment, n in any one of the O-antigen Formulas is at least 75 and at most 100. In one embodiment, n in any one of the O-antigen Formulas is at least 1 and at most 75. In one embodiment, n in any one of the O-antigen Formulas is at least 5 and at most 75. In one embodiment, n in any one of the O-antigen Formulas is at least 10 and at most 75. In one embodiment, n in any one of the O-antigen Formulas is at least 20 and at most 75. In one embodiment, n in any one of the O-antigen Formulas is at least 25 and at most 75. In one embodiment, n in any one of the O-antigen Formulas is at least 30 and at most 75. In one embodiment, n in any one of the O-antigen Formulas is at least 40 and at most 75. In one embodiment, n in any one of the O-antigen Formulas is at least 50 and at most 75. In one embodiment, n in any one of the O-antigen Formulas is at least 30 and at most 90. In one embodiment, n in any one of the O-antigen Formulas is at least 35 and at most 85. In one embodiment, n in any one of the O-antigen Formulas is at least 35 and at most 75. In one embodiment, n in any one of the O-antigen Formulas is at least 35 and at most 70. In one embodiment, n in any one of the O-antigen Formulas is at least 35 and at most 60. In one embodiment, n in any one of the O-antigen Formulas is at least 35 and at most 50. In one embodiment, n in any one of the O-antigen Formulas is at least 35 and at most 49. In one embodiment, n in any one of the O-antigen Formulas is at least 35 and at most 48. In one embodiment, n in any one of the O-antigen Formulas is at least 35 and at most 47. In one embodiment, n in any one of the O-antigen Formulas is at least 35 and at most 46. In one embodiment, n in any one of the O-antigen Formulas is at least 36 and at most 45. In one embodiment, n in any one of the O-antigen Formulas is at least 37 and at most 44. In one embodiment, n in any one of the O-antigen Formulas is at least 38 and at most 43. In one embodiment, n in any one of the O-antigen Formulas is at least 39 and at most 42. In one embodiment, n in any one of the O-antigen Formulas is at least 39 and at most 41.

For example, in one embodiment, n in the saccharide is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, most preferably 40. In another embodiment, n is at least 35 to at most 60. For example, in one embodiment, n is any one of 35, 36, 37, 38, 39, 40, 41, 42, 43, 44, 45, 46, 47, 48, 49, 50, 51, 52, 53, 54, 55, 56, 57, 58, 59, and 60, preferably 50. In another preferred embodiment, n is at least 55 to at most 75. For example, in one embodiment, n is 55, 56, 57, 58, 59, 60, 61, 62, 63, 64, 65, 66, 67, 68, or 69, most preferably 60.

The saccharide structure may be determined by methods and tools known art, such as, for example, NMR, including 1 D, 1H, and/or 13C, 2D TOCSY, DQF-COSY, NOESY, and/or HMQC.

In some embodiments, the purified polysaccharide before conjugation has a molecular weight of between 5 kDa and 400 kDa. In other such embodiments, the saccharide has a molecular weight of between 10 kDa and 400 kDa; between 5 kDa and 400 kDa; between 5 kDa and 300 kDa; between 5 kDa and 200 kDa; between 5 kDa and 150 kDa; between 10 kDa and 100 kDa; between 10 kDa and 75 kDa; between 10 kDa and 60 kDa; between 10 kDa and 40 kDa; between 10 kDa and 100 kDa; 10 kDa and 200 kDa; between 15 kDa and 150 kDa; between 12 kDa and 120 kDa; between 12 kDa and 75 kDa; between 12 kDa and 50 kDa; between 12 and 60 kDa; between 35 kDa and 75 kDa; between 40 kDa and 60 kDa; between 35 kDa and 60 kDa; between 20 kDa and 60 kDa; between 12 kDa and 20 kDa; or between 20 kDa and 50 kDa. In further embodiments, the polysaccharide has a molecular weight of between 7 kDa to 15 kDa; 8 kDa to 16 kDa; 9 kDa to 25 kDa; 10 kDa to 100; 10 kDa to 60 kDa; 10 kDa to 70 kDa; 10 kDa to 160 kDa; 15 kDa to 600 kDa; 20 kDa to 1000 kDa; 20 kDa to 600 kDa; 20 kDa to 400 kDa; 30 kDa to 1,000 KDa; 30 kDa to 60 kDa; 30 kDa to 50 kDa or 5 kDa to 60 kDa. Any whole number integer within any of the above ranges is contemplated as an embodiment of the disclosure.

As used herein, the term “molecular weight” of polysaccharide or of carrier protein-polysaccharide conjugate refers to molecular weight calculated by size exclusion chromatography (SEC) combined with multiangle laser light scattering detector (MALLS).

A polysaccharide can become slightly reduced in size during normal purification procedures. Additionally, as described herein, polysaccharide can be subjected to sizing techniques before conjugation. Mechanical or chemical sizing maybe employed. Chemical hydrolysis may be conducted using acetic acid. Mechanical sizing may be conducted using High Pressure Homogenization Shearing. The molecular weight ranges mentioned above refer to purified polysaccharides before conjugation (e.g., before activation).

TABLE 1
E. coli serogroups/serotypes and O-unit moieties

| | | Moiety |
|---|---|---|
| | | structure |
| Serogroup/ | | referred to |
| Serotype | Moiety Structure (O-unit) | herein as: |
| O1A, O1A1 | [→3)-α-L-Rha-(1→3)-α-L-Rha-(1→3)-β-L-Rha-(1→4)-β-D- | Formula O1A |
| | GlNAc-(1→ \| β-D-ManNAc-(1→2) ]ₙ | |
| O1B | [→3)-α-L-Rha-(1→2)-α-L-Rha-(1→2)-α-D-Gal-(1→3)-β-D- | Formula O1B |
| | GlcNAc-(1→\|β-D-ManNAc-(1→2) ]ₙ | |
| O1C | [→3)-α-L-Rha-(1→2)-α-L-Rha-(1→3)-α-D-Gal-(1→3)-β-D- | Formula O1C |
| | GlcNAc-(1→\|β-D-ManNAc-(1→2) ]ₙ | |
| O2 | [→3)-α-L-Rha-(1→2)-α-L-Rha-(1→3)-β-L-Rha-(1→4)-β-D- | Formula O2 |
| | GlcNAc-(1→ \| α-D-Fuc3NAc-(1→2) ]ₙ | |
| O3 | [β-L-RhaNAc(1→4)α-D-Glc-(1→4)\| \| →3)-β-D-GlcNAc-(1→3)- | Formula O3 |
| | α-D-Gal-(1→3)-β-D-GlcNAc-(1→ ]ₙ | |
| O4:K52 | [→2)-α-L-Rha-(1→6)-α-D-Glc-(1→3)-α-L-FucNAc-(1→3)-β-D- | Formula |
| | GlcNAc(1→ ]ₙ | O4:K52 |
| O4:K6 | [α-D-Glc-(1→3) \| →2)-α-L-Rha-(1→6)-α-D-Glc-(1→3)-α-L- | Formula |
| | FucNAc-(1→3)-β-D-GlcNAc(1→ ]ₙ | O4:K6 |
| O5ab | [→4)-β-D-Qui3NAc-(1→3)-β-D-Ribf-(1→4)-β-D-Gal-(1→3)-α- | Formula |
| | D-GalNAc(1→]ₙ | O5ab |
| O5ac (strain | [→2)-β-D-Qui3NAc-(1→3)-β-D-Ribf-(1→4)-β-D-Gal-(1→3)-α- | Formula |
| 180/C3) | D-GalNAc(1→ ]ₙ | O5ac (strain |
| | | 180/C3) |
| O6:K2; | [→4)-α-D-GalNAc-(1→3)-β-D-Man-(1→4)-β-D-Man-(1→3)-α- | Formula |
| K13; K15 | D-GlcNAc-(1→ \| β-D-Glc-(1→2) ]ₙ | O6:K2; |
| | | K13; K15 |
| O6:K54 | [→4)-α-D-GalNAc-(1→3)-β-D-Man-(1→4)-β-D-Man-(1→3)-α- | Formula |
| | D-GlcNAc-(1→\|β-D-GlcNAc-(1→2) ]ₙ | O6:K54 |
| O7 | [α-L-Rha-(1→3) \| →3)-β-D-Qui4NAc-(1→2)-α-D-Man-(1→4)-β- | Formula O7 |
| | D-Gal-(1→3)-α-D-GlcNAc-(1→ ]ₙ | |
| O10 | [→3)-α-L-Rha-(1→3)-α-L-Rha-(1→3)-α-D-Gal-(1→3)-β-D- | Formula O10 |
| | GlcNAc-(1→ \| α-D-Fuc4NAcyl-(1→2) Acyl = acetyl (60%) or | |
| | (R)-3-hydroxybutyryl (40%) ]ₙ | |
| O16 | [→2)-β-D-Galf-(1→6)-α-D-Glc-(1→3)-α-L-Rha2Ac-(1→3)-α-D- | Formula O16 |
| | GlcNAc-(1→ ]ₙ | |
| O17 | [α-D-Glc-(1→6) \| →6)-α-D-Man-(1→2)-α-D-Man-(1→2)-β-D- | Formula O17 |
| | Man-(1→3)-α-D-GlcNAc(1→ ]ₙ | |
| O18A, O18ac | [→2)-α-L-Rha-(1→6)-α-D-Glc-(1→4)-α-D-Gal-(1→3)-α-D- | Formula |
| | GlcNAc-(1→ \| β-D-GlcNAc-(1→3) ]ₙ | O18A, |
| | | Formula |
| | | O18ac |
| O18A1 | [α-D-Glc-(1→6) \| →2)-α-L-Rha-(1→6)-α-D-Glc-(1→4)-α-D-Gal- | Formula |
| | (1→3)-α-D-GlcNAc-(1→ \| β-D-GlcNAc-(1→3) ]ₙ | O18A1 |
| O18B | [→3)-α-L-Rha-(1→6)-α-D-Glc-(1→4)-α-D-Gal-(1→3)-α-D- | Formula |
| | GlcNAc-(1→ \| B-D-Glc-(1→3) ]ₙ | O18B |
| O18B1 | [α-D-Glc-(1→4) \| →3)-α-L-Rha-(1→6)-α-D-Glc-(1→4)-α-D-Gal- | Formula |
| | (1→3)-α-D-GlcNAc-(1→ \| β-D-Glc-(1→3) ]ₙ | O18B1 |
| O21 | [β-D-Gal-(1→4) \| →3)-β-D-Gal-(1→4)-β-D-Glc-(1→3)-β-D- | Formula O21 |
| | GalNAc-(1→ \| β-D-GlcNAc-(1→2) ]ₙ | |
| O23A | [α-D-Glc-(1→6) \| →6)-α-D-Glc-(1→4)-β-D-Gal-(1→3)-α-D- | Formula |
| | GalNAc-(1→3)-β-D-GlcNAc-(1→ \| β-D-GlcNAc(1→3) ]ₙ | O23A |
| O24 | [→7)-α-Neu5Ac-(2→3)-β-D-Glc-(1→3)-β-D-GalNAc-(1→ \| α-D- | Formula O24 |
| | Glc-(1→2) ]ₙ | |
| O25/O25a | [β-D-Glc-(1→6) \| →4)-α-D-Glc-(1→3)-α-L-FucNAc-(1→3)-β-D- | Formula |
| | GlcNAc-(1→ \| α-L-Rha-(1→3) ]ₙ | O25a |
| O25b | 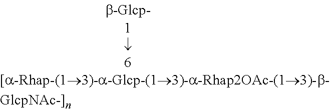 | Formula O25b |
| O26 | [ →3)-α-L-Rha-(1→4)-α-L-FucNAc-(1→3)-β-D-GlcNAc-(1→ ]ₙ | Formula O26 |
| O28 | [ →2)-(R)-Gro-1-P→4)-β-D-GlcNAc-(1→3)-β-D-Galf2Ac-(1→3)- | Formula O28 |
| | α-D-GlcNAc-(1→ ]ₙ | |
| O36 | 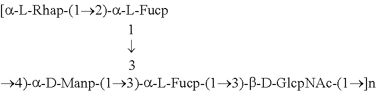 | Formula O36 |
| O44 | [ α-D-Glc-(1→4) \| →6)-α-D-Man-(1→2)-α-D-Man-(1→2)-β-D- | Formula O44 |
| | Man-(1→3)-α-D-GlcNAc(1→ ]ₙ | |
| O45 | [→2)-β-D-Glc-(1→3)-α-L-6dTal2Ac-(1→3)-α-D-FucNAc-(1→ ]ₙ | Formula O45 |
| O45rel | [→2)-β-D-Glc-(1→3)-α-L-6dTal2Ac-(1→3)-β-D-GlcNAc-(1→ ]ₙ | Formula |
| | | O45rel |
| O54 | [→4)-α-d-GalpA-(1 → 2)-α-1-Rhap-(1 → 2)-β-d-Ribf- | Formula O54 |
| | (1 → 4)-β-d-Galp-(1 → 3)-β-d-GlcpNAc-(1→]n | |
| O55 | [ →6)-β-D-GlcNAc-(1→3)-α-D-Gal-(1→3)-β-D-GalNAc-(1→ \| α- | Formula O55 |
| | Col-(1→2)-β-D-Gal-(1→3) ]ₙ | |
| O56 | [ →7)-α-Neu5Ac-(2→3)-β-D-Glc-(1→3)-β-D-GlcNAc-(1→ \| α-D- | Formula O56 |
| | Gal-(1→2) ]ₙ | |
| O57 | 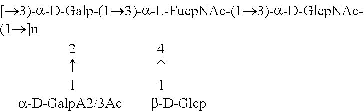 | Formula O57 |
| O58 | [ 3-O-[(R)-1-carboxyethyl]-α-L-Rha-(1→3) \| →4)-α-D-Man- | Formula O58 |
| | (1→4)-α-D-Man2Ac-(1→3)-β-D-GlcNAc-(1→ ]ₙ | |
| O64 | [ β-D-Gal-(1→6) \| →3)-α-D-ManNAc-(1→3)-β-D-GlcA-(1→3)-β- | Formula O64 |
| | D-Gal-(1→3)-β-D-GlcNAc(1→ ]ₙ | |
| O68 | 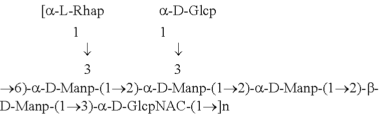 | Formula O68 |
| O69 | [ →2)-α-L-Rha-(1→2)-α-L-Rha-(1→2)-α-D-Gal-(1→3)-β-D- | Formula O69 |
| | GlcNAc-(1→ ]ₙ | |
| O73 (Strain | [ α-D-Glc-(1-3) \| →4)-α-D-Man-(1→2)-α-D-Man-(1→2)-β-D- | Formula O73 |
| 73-1) | Man-(1→3)-α-D-GalNAc(1→ ]ₙ | (Strain 73-1) |
| O74 | 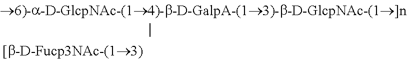 | Formula O74 |
| O75 | [ β-D-Man-(1→4) \| →3)-α-D-Gal-(1→4)-α-L-Rha-(1→3)-β-D- | Formula O75 |
| | GlcNAc-(1→ ]ₙ | |
| O76 | [→4)-β-D-GlCpA-(1→4)-β-D-GalpNAc3Ac-(1→4)-α-D- | Formula O76 |
| | GalpNAc-(1→3)-β-D-GalpNAc-(1→]ₙ | |
| O77 | [ →6)-α-D-Man-(1→2)-α-D-Man-(1→2)-β-D-Man-(1→3)-α-D- | Formula O77 |
| | GlcNAc(1→ ]ₙ | |
| O78 | [ →4)-β-D-GlcNAc-(1→4)-β-D-Man-(1→4)-α-D-Man-(1→3)-β- | Formula O78 |
| | D-GlcNAc-(1→ ]ₙ | |
| O86 | [ α-D-Gal-(1→3) \| →4)-α-L-Fuc-(1→2)-β-D-Gal-(1→3)-α-D- | Formula O86 |
| | GalNAc-(1→3)-β-D-GalNAc-(1→ ]ₙ | |
| O88 | [ α-L-6dTal-(1→3) \| →4)-α-D-Man-(1→3)-α-D-Man-(1→3)-β-D- | Formula O88 |
| | GlcNAc-(1→ ]ₙ | |
| O90 | [ →4)-α-L-Fuc2/3Ac-(1→2)-β-D-Gal-(1→3)-α-D-GalNAc- | Formula O90 |
| | (1→3)-β-D-GalNAc-(1→ ]ₙ | |
| O98 | [ →3)-α-L-QuiNAc-(1→4)-α-D-GalNAcA-(1→3)-α-L-QuiNAc- | Formula O98 |
| | (1→3)-β-D-GlcNAc-(1→ ]ₙ | |
| O104 | [ →4)-α-D-Gal-(1→4)-α-Neu5,7,9Ac₃-(2→3)-β-D-Gal-(1→3)-β- | Formula |
| | D-GalNAc-(1→]ₙ | O104 |
| O111 | [ α-Col-(1→6) \| →4)-α-D-Glc-(1→4)-α-D-Gal-(1→3)-β-D- | Formula |
| | GlcNAc-(1→ \| α-Col-(1→3) ]ₙ | O111 |
| O113 | [ →4)-α-D-GalNAc-(1→4)-α-D-GalA-(1→3)-α-D-Gal-(1→3)-β- | Formula |
| | D-GlcNAc-(1→ \| β-D-Gal-(1→3) ]ₙ | O113 |
| O114 | [ →4)-β-D-Qui3N(N-acetyl-L-seryl)-(1→3)-β-D-Ribf-(1→4)-β- | Formula |
| | D-Gal-(1→3)-α-D-GlcNAc(1→ ]ₙ | O114 |
| O119 | [ β-D-RhaNAc3NFo-(1→3) \| →2)-β-D-Man-(1→3)-α-D-Gal- | Formula |
| | (1→4)-α-L-Rha-(1→3)-α-D-GlcNAc-(1→ ]ₙ | O119 |
| O121 | [ →3)-β-D-Qui4N(N-acetyl-glycyl)-(1→4)-α-β- | Formula |
| | GalNAc3AcA6N-(1→4)-α-D-GalNAcA-(1→3)-α-D-GlcNAc- | O121 |
| | (1→ ]ₙ | |
| O124 | [ 4-O-[(R)-1-carboxyethyl]-β-D-Glc-(1→6)-α-D-Glc(1→4) | Formula |
| | \|→3)-α-D-Gal-(1→6)-β-D-Galf-(1→3)-β-D-GalNAc-(1→ ]ₙ | O124 |
| O125 | [ α-D-Glc-(1→3) \| →4)-β-D-GalNAc-(1→2)-α-D-Man-(1→3)-α- | Formula |
| | L-Fuc-(1→3)-α-D-GalNAc-(1→ \| β-D-Gal-(1→3) ]ₙ | O125 |
| O126 | [ →2)-β-D-Man-(1→3)-β-D-Gal-(1→3)-α-D-GlcNAc-(1→3)-β-D- | Formula |
| | GlcNAc-(1→ \| α-L-Fuc-(1→2) ]ₙ | O126 |
| O127 | [ →2)-α-L-Fuc-(1→2)-β-D-Gal-(1→3)-α-D-GalNAc-(1→3)-α-D- | Formula |
| | GalNAc-(1→ ]ₙ | O127 |
| O128 | [ α-L-Fuc-(1→2) \| →6)-β-D-Gal-(1→3)-β-D-GalNAc-(1→4)-α-D- | Formula |
| | Gal-(1→3)-β-D-GalNAc-(1→ ]ₙ | O128 |
| O136 | [ →4)-β-Pse5Ac7Ac-(2→4)-β-D-Gal-(1→4)-β-D-GlNAc-(1→β- | Formula |
| | Pse5Ac7Ac = 5,7-diacetamido-3,5,7,9-tetradeoxy-L- | O136 |
| | glycero-β-L-manno-nonulosonic acid ]ₙ | |
| O138 | [ →2)-α-L-Rha-(1→3)-α-L-Rha-(1→4)-α-D-GalNAcA-(1→3)-β- | Formula |
| | D-GlcNAc-(1→ ]ₙ | O138 |
| O140 | 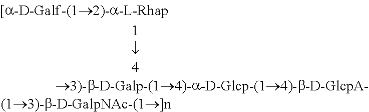 | Formula O140 |
| O141 | [ α-L-Rha-(1→3) \|→4)-α-D-Man-(1→3)-α-D-Man6Ac-(1→3)-β- | Formula |
| | D-GlcNAc-(1→ \| β-D-GlcA-(1→2) ]ₙ | O141 |
| O142 | [ →2)-α-L-Rha-(1→6)-α-D-GalNAc-(1→4)-α-D-GalNAc-(1→3)- | Formula |
| | α-D-GalNAc-(1→ \| β-D-GlcNAc-(1→3) ]ₙ | O142 |
| O143 | [ →2)-β-D-GalA6R3,4Ac-(1→3)-α-D-GalNAc-(1→4)-β-D-GlcA- | Formula |
| | (1→3)-β-D-GlcNAc-(1→ R = 1,3-dihydroxy-2-propylamino ]ₙ | O143 |
| O147 | [ →2)-α-L-Rha-(1→2)-α-L-Rha-(1→4)-β-D-GalA-(1→3)-β-D- | Formula |
| | GalNAc-(1→ ]ₙ | O147 |
| O149 | [ →3)-β-D-GlcNAc-(S)-4,6Py-(1→3)-β-L-Rha-(1→4)-β-D- | Formula |
| | GlcNAc-(1→ (S)-4,6Py = 4,6-O-[(S)-1-carboxyethylidene]- ]ₙ | O149 |
| O152 | [ β-L-Rha-(1→4) \| →3)-α-D-GlcNAc-(1-P→6)-α-D-Glc-(1→2)-β- | Formula |
| | D-Glc-(1→3)-β-D-GlcNAc-(1→ ]ₙ | O152 |
| O157 | [ →2)-α-D-Rha4NAc-(1→3)-α-L-Fuc-(1→4)-β-D-Glc-(1→3)-α- | Formula |
| | D-GalNAc-(1→ ]ₙ | O157 |
| O158 | [ α-D-Glc-(1→6) \| →4)-α-D-Glc-(1→3)-α-D-GalNAc-(1→3)-β-D- | Formula |
| | GalNAc-(1→ \| α-L-Rha-(1→3) ]ₙ | O158 |
| O159 | [ α-L-Fuc-(1→4) \| →3)-β-D-GlcNAc-(1→4)-α-D-GalA-(1→3)-α- | Formula |
| | L-Fuc-(1→3)-β-D-GlcNAc-(1→ ]ₙ | O159 |
| O164 | [ β-D-Glc-(1→6)-α-D-Glc(1→4) \| →3)-β-D-Gal-(1→6)-β-D-Galf- | Formula |
| | (1→3)-β-D-GalNAc-(1→ ]ₙ | O164 |
| O173 | [ α-L-Fuc-(1→4) \| →3)-α-D-Glc-(1-P→6)-α-D-Glc-(1→2)-β-D- | Formula |
| | Glc-(1→3)-β-D-GlcNAc-(1→]ₙ | O173 |
| 62D₁ | [ α-D-Gal(1→6) \| →2)-β-D-Qui3NAc-(1→3)-α-L-Rha-(1-3)-β- | Formula |
| Suggested as | D-Gal-(1→3)-α-D-FucNAc-(1→ ]ₙ | 62D₁ |
| Erwinia | | |
| herbicola | | |
| O22 | [ →6)-α-D-Glc-(1→4)-β-D-GlcA-(1→4)-β-D-GalNAc3Ac-(1→3)- | Formula O22 |
| | α-D-Gal-(1→3)-β-D-GalNAc-(1→]ₙ | |
| O35 | [ →3)-α-L-Rha-(1→2)-α-L-Rha-(1→3)-α-L-Rha-(1→2)-α-L- | Formula O35 |
| | Rha-(1→3)-β-D-GlcNAc-(1→ \| α-D-GalNAcA6N-(1→2) ]ₙ | |
| O65 | [ →2)-β-D-Qui3NAc-(1→4)-α-D-GalA6N-(1→4)-α-D-GalNAc- | Formula O65 |
| | (1→4)-β-D-GalA-(1→3)-α-D-GlcNAc-(1→ ]ₙ | |
| O66 | [ →2)-β-D-Man-(1→3)-α-D-GlcNAc-(1→2)-β-D-Glc3Ac-(1→3)- | Formula O66 |
| | α-L-6dTal-(1→3)-α-D-GlcNAc(1→ ]ₙ | |
| O83 | [ →6)-α-D-Glc-(1→4)-β-D-GlcA-(1→6)-β-D-Gal-(1→4)-β-D- | Formula O83 |
| | Gal-(1→4)-β-D-GlcNAc-(1→ ]ₙ | |
| O91 | [ →4)-α-D-Qui3NAcyl-(1→4)-β-D-Gal-(1→4)-β-D-GlcNAc- | Formula O91 |
| | (1→4)-β-D-GlcA6NGly-(1→3)-β-D-GlcNAc-(1→ Acyl = (R)-3- | |
| | hydroxybutyryl ]ₙ | |
| O105 | [ β-D-Ribf-(1→3) \|→4)-α-D-GlcA2Ac3Ac-(1→2)-α-L-Rha4Ac- | Formula |
| | (1→3)-β-L-Rha-(1→4)-β-L-Rha-(1→3)-β-D-GlcNAc6Ac-(1→ ]ₙ | O105 |
| O116 | [ →2)-β-D-Qui4NAc-(1→6)-α-D-GlcNAc-(1→4)-α-D-GalNAc- | Formula |
| | (1→4)-α-D-GalA-(1→3)-β-D-GlcNAc-(1→ ]ₙ | O116 |
| O117 | [ →4)-β-D-GalNAc-(1→3)-α-L-Rha-(1→4)-α-D-Glc-(1→4)-β-D- | Formula |
| | Gal-(1→3)-α-D-GalNAc-(1→]ₙ | O117 |
| O139 | [ β-D-Glc-(1→3) \| →3)-α-L-Rha-(1→4)-α-D-GalA-(1→2)-α-L- | Formula |
| | Rha-(1→3)-α-L-Rha-(1→2)-α-L-Rha-(1→3)-α-D-GlcNAc-(1→ ]ₙ | O139 |
| O153 | [ →2)-β-D-Ribf-(1→4)-β-D-Gal-(1→4)-α-D-GlcNAc-(1→4)-β-D- | Formula |
| | Gal-(1→3)-α-D-GlcNAc-(1→ ]ₙ | O153 |
| O167 | [ α-D-Galf-(1→4) \| →2)-β-D-GalA6N(L)Ala-(1→3)-α-D-GlcNAc- | Formula |
| | (1→2)-β-D-Galf-(1→5)-β-D-Galf-(1→3)-β-D-GlcNAc-(1→ ]ₙ | O167 |
| O172 | [ →3)-α-L-FucNAc-(1→4)-α-D-Glc6Ac-(1-P→4)-α-D-Glc- | Formula |
| | (1→3)-α-L-FucNAc-(1→3)-α-D-GlcNAc-(1→ ]ₙ | O172 |
| O8 | [ →2)-α-D-Man-(1→2)-α-D-Man-(1→3)-β-D-Man-(1→ ]ₙ | Formula O8 |
| O9a | [ →2)-α-D-Man-(1→2)-α-D-Man-(1→3)-α-D-Man-(1→3)- | Formula O9a |
| | α-D-Man-(1→ ]ₙ | |
| O9 | [ →2)-[α-D-Man-(1→2)]₂-α-D-Man-(1→3)-α-D-Man- | Formula O9 |
| | (1→3)-α-D-Man-(1→ ]ₙ | |
| O20ab | [ →2)-β-D-Ribf-(1→4)-α-D-Gal-(1→ ]ₙ | Formula |
| | | O20ab |
| O20ac | [ α-D-Gal-(1→3) \| →2)-β-D-Ribf-(1→4)-α-D-Gal-(1→ ]ₙ | Formula |
| | | O20ac |
| O52 | [ →3)-β-D-Fucf-(1→3)-β-D-6dmanHep2Ac-(1→ ]ₙ | Formula O52 |
| O97 | [ →3)-α-L-Rha-(1→3)-β-L-Rha-(1→ \| \| β-D-Xulf-(2→2)β- | Formula O97 |
| | D-Xulf-(2→2) ]ₙ | |

† β-D-6dmanHep2Ac is 2-O-acetyl-6-deoxy-β-D-manno-heptopyranosyl.
‡ β-D-Xulf is β-D-threo-pentofuranosyl.

### Core Oligosaccharide

The core oligosaccharide is positioned between Lipid A and the O-antigen outer region in wild-type E. coli LPS. More specifically, the core oligosaccharide is the part of the polysaccharide that includes the bond between the O-antigen and the lipid A in wild type E. coli. This bond includes a ketosidic bond between the hemiketal function of the innermost 3-deoxy-d-manno-oct-2-ulosonic acid (KDO)) residue and a hydroxyl-group of a GlcNAc-residue of the lipid A. The core oligosaccharide region shows a high degree of similarity among wild-type E. coli strains. It usually includes a limited number of sugars. The core oligosaccharide includes an inner core region and an outer core region.

More specifically, the inner core is composed primarily of L-glycero-D-manno-heptose (heptose) and KDO residues. The inner core is highly conserved. A KDO residue includes the following Formula KDO:

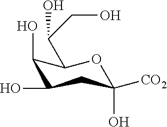

The outer region of the core oligosaccharide displays more variation than the inner core region, and differences in this region distinguish the five chemotypes in E. coli: R1, R2, R3, R4, and K-12. HepII is the last residue of the inner core oligosaccharide. While all of the outer core oligosaccharides share a structural theme, with a (hexose)₃ carbohydrate backbone and two side chain residues, the order of hexoses in the backbone and the nature, position, and linkage of the side chain residues can all vary. The structures for the R1 and R4 outer core oligosaccharides are highly similar, differing in only a single β-linked residue.

The core oligosaccharides of wild-type E. coli are categorized in the art based on the structures of the distal oligosaccharide, into five different chemotypes: E. coli R1, E. coli R2, E. coli R3, E. coli R4, and E. coli K12.

In a preferred embodiment, the compositions described herein include glycoconjugates in which the O-polysaccharide includes a core oligosaccharide bound to the O-antigen. In one embodiment, the composition induces an immune response against at least any one of the core E. coli chemotypes E. coli R1, E. coli R2, E. coli R3, E. coli R4, and E. coli K12. In another embodiment, the composition induces an immune response against at least two core E. coli chemotypes. In another embodiment, the composition induces an immune response against at least three core E. coli chemotypes. In another embodiment, the composition induces an immune response against at least four core E. coli chemotypes. In another embodiment, the composition induces an immune response against all five core E. coli chemotypes.

In another preferred embodiment, the compositions described herein include glycoconjugates in which the O-polysaccharide does not include a core oligosaccharide bound to the O-antigen. In one embodiment, such a composition induces an immune response against at least any one of the core E. coli chemotypes E. coli R1, E. coli R2, E. coli R3, E. coli R4, and E. coli K12, despite the glycoconjugate having an O-polysaccharide that does not include a core oligosaccharide.

E. coli serotypes may be characterized according to one of the five chemotypes. Table 2 lists exemplary serotypes characterized according to chemotype. The serotypes in bold represent the serotypes that are most commonly associated with the indicated core chemotype. Accordingly, in a preferred embodiment, the composition induces an immune response against at least any one of the core E. coli chemotypes E. coli R1, E. coli R2, E. coli R3, E. coli R4, and E. coli K12, which includes an immune response against any one of the respective corresponding E. coli serotypes.

TABLE 2
Core Chemotype and associated E. coli Serotype

| Core chemotype | Serotype |
|---|---|
| R1 | O25a, O6, O2, O1, O75, O4, O16, O8, O18, |
| | O9, O13, O20, O21, O91, and O163. |
| R2 | O21, O44, O11, O89, O162, O9 |
| R3 | O25b, O15, O153, O21, O17, O11, O159, |
| | O22 O86, O93 |
| R4 | O2, O1, O86, O7, O102, O160, O166 |
| K-12 | O25b, O16 |

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O8, Formula O18, Formula O9, Formula O13, Formula O20, Formula O21, Formula O91, and Formula O163, wherein n is 1 to 100. In some embodiments, the saccharide in said composition further includes an E. coli R1 core moiety.

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O18, Formula O13, Formula O20, Formula O21, Formula O91, and Formula O163, wherein n is 1 to 100, preferably 31 to 100, more preferably 35 to 90, most preferably 35 to 65. In some embodiments, the saccharide in said composition further includes an E. coli R1 core moiety in the saccharide.

In some embodiments, the composition includes a saccharide that includes a structure derived from a serotype having an R2 chemotype, e.g., selected from a saccharide having Formula O21, Formula O44, Formula O11, Formula O89, Formula O162, and Formula O9, wherein n is 1 to 100, preferably 31 to 100, more preferably 35 to 90, most preferably 35 to 65.

In some embodiments, the saccharide in said composition further includes an E. coli R2 core moiety.

In some embodiments, the composition includes a saccharide that includes a structure derived from a serotype having an R3 chemotype, e.g., selected from a saccharide having Formula O25b, Formula O15, Formula O153, Formula O21, Formula O17, Formula O11, Formula O159, Formula O22, Formula O86, and Formula O93, wherein n is 1 to 100, preferably 31 to 100, more preferably 35 to 90, most preferably 35 to 65. In some embodiments, the saccharide in said composition further includes an E. coli R3 core moiety.

In some embodiments, the composition includes a saccharide that includes a structure derived from a serotype having an R4 chemotype, e.g., selected from a saccharide having Formula O2, Formula O1, Formula O86, Formula O7, Formula O102, Formula O160, and Formula O166, wherein n is 1 to 100, preferably 31 to 100, more preferably 35 to 90, most preferably 35 to 65. In some embodiments, the saccharide in said composition further includes an E. coli R4 core moiety.

In some embodiments, the composition includes a saccharide that includes a structure derived from a serotype having an K-12 chemotype (e.g., selected from a saccharide having Formula O25b and a saccharide having Formula O16), wherein n is 1 to 1000, preferably 31 to 100, more preferably 35 to 90, most preferably 35 to 65. In some embodiments, the saccharide in said composition further includes an E. coli K-12 core moiety.

In some embodiments, the saccharide includes the core saccharide. Accordingly, in one embodiment, the O-polysaccharide further includes an E. coli R1 core moiety. In another embodiment, the O-polysaccharide further includes an E. coli R2 core moiety. In another embodiment, the O-polysaccharide further includes an E. coli R3 core moiety. In another embodiment, the O-polysaccharide further includes an E. coli R4 core moiety. In another embodiment, the O-polysaccharide further includes an E. coli K12 core moiety.

In some embodiments, the saccharide does not include the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety.

Saccharide and/or Polypeptide or Fragments Thereof Derived from Klebsiella Pneumoniae

Klebsiella pneumoniae is a Gram-negative pathogen, known to cause urinary tract infections, bacteremia, and sepsis. In one aspect, any of the compositions disclosed herein may further include at least one saccharide that is, or derived from, at least one K. pneumoniae serotype selected from O1 (and d-Gal-III variants), O2 (and d-Gal-III variants), O2ac, O3, O4, O5, O7, O8, and O12. In a preferred embodiment, any of the compositions disclosed herein may further include a polypeptide derived from K. pneumoniae selected from a polypeptide derived from K. pneumoniae Type I fimbrial protein or an immunogenic fragment thereof; and a polypeptide derived from K. pneumoniae Type III fimbrial protein or an immunogenic fragment thereof.

As is known in the art, K. pneumoniae O1 and O2 antigens contain homopolymer galac-tose units (or galactans). K. pneumoniae O1 and O2 antigens each contain D-galactan I units (sometimes referred to as the O2a repeat unit), but O1 antigens differ in that O1 antigens have a D-galactan II cap structure. D-galactan III (d-Gal-III) is a variant of D-galactan I. In some embodiments, the saccharide derived from K. pneumoniae O1 includes a repeat unit of [→3)-β-D-Galf-(1→3)-α-D-Galp-(1→]. In some embodiments, the saccharide derived from K. pneumoniae O1 includes a repeat unit of [→3)-α-D-Galp-(1→3)-β-D-Galp-(1→]. In some embodiments, the saccharide derived from K. pneumoniae O1 includes a repeat unit of [→3)-β-D-Galf-(1→3)-α-D-Galp-(1→], and a repeat unit of [→3)-α-D-Galp-(1→3)-β-D-Galp-(1→]. In some embodiments, the saccharide derived from K. pneumoniae O1 includes a repeat unit of →3)-β-D-Galf-(1→3)-[α-D-Galp-(1→4)]-α-D-Galp-(1→] (referred to as the D-Gal-III repeat unit).

In some embodiments, the saccharide derived from K. pneumoniae O2 includes a repeat unit of [→3)-α-D-Galp-(1→3)-β-D-Galf-(1→] (which may be an element of K. pneumoniae serotype O2a antigen). In some embodiments, the saccharide derived from K. pneumoniae O2 includes a repeat unit of [→3)-β-D-GlcpNAc-(1→5)-β-D-Galf-(1→](which may be an element of K. pneumoniae serotype O2c antigen). In some embodiments, the saccharide derived from K. pneumoniae O2 includes a modification of the O2a repeat unit by side chain addition of (1→4)-linked Galp residues (which may be an element of the K. pneumoniae O2afg antigen). In some embodiments, the saccharide derived from K. pneumoniae O2 includes a modification of the O2a repeat unit by side chain addition of (1→2)-linked Galp residues (which may be an element of the K. pneumoniae O2aeh antigen).

Without being bound by mechanism or theory, O-antigen polysaccharide structure of K. pneumoniae serotypes O3 and O5 are disclosed in the art to be identical to those of E. coli serotypes O9a (Formula O9a) and O8 (Formula O8), respectively.

In some embodiments, the saccharide derived from K. pneumoniae O4 includes a repeat unit of [→4)-α-D-Galp-(1→2)-β-D-Ribf-(1→)]. In some embodiments, the saccharide derived from K. pneumoniae O7 includes a repeat unit of [→2-α-L-Rhap-(1→2)-β-D-Ribf-(1→3)-α-L-Rhap-(1→3)-α-L-Rhap-(1→]. In some embodiments, the saccharide derived from K. pneumoniae O8 serotype includes the same repeat-unit structure as K. pneumoniae O2a, but is nonstoichiometrically O-acetylated. In some embodiments, the saccharide derived from K. pneumoniae O12 serotype includes a repeat unit of [α-Rhap-(1→3)-β-GlcpNAc] disaccharide repeat unit.

TABLE 1a
K. pneumoniae serogroups/serotypes and O-unit moieties

| | | Moiety |
|---|---|---|
| | | structure |
| Serogroup/ | | referred to |
| Serotype | Moiety Structure (O-unit) | herein as: |
| O1 | [→3)-β-D-Galf-(1→3)-α-D-Galp-(1→]ₙ | Formula |
| | | K.O1.1 |
| O1 | [→3)-α-D-Galp-(1→3)-β-D-Galp-(1→]ₙ | Formula |
| | | K.O1.2 |
| O1 | [→3)-β-D-Galf-(1→3)-α-D-Galp-(1→]ₙ and | Formula |
| | [→3)-α-D-Galp-(1→3)-β-D-Galp-(1→]ₙ | K.O1.3 |
| O1 | [→3)-β-D-Galf-(1→3)- | Formula |
| | [α-D-Galp-(1→4)]-α-D-Galp-(1→]ₙ | K.O1.4 |
| O2 | [→3)-α-_{D}-Galp-(1→3)-β-_{D}-Galf-(1→]ₙ | Formula |
| | | K.O2.1 |
| O2 | [→3)-β-_{D}-GlcpNAc-(1→5)-β-_{D}-Galf-(1→]ₙ | Formula |
| | | K.O2.2 |
| O2 | Modified [→3)-α-_{D}-Galp-(1→3)- | Formula |
| | β-_{D}-Galf-(1→]ₙ by side | K.O2.3 |
| | chain addition of (1→4)-linked Galp residues | |
| O2 | Modified [→3)-α-_{D}-Galp-(1→3)- | Formula |
| | β-_{D}-Galf-(1→]ₙ by side | K.O2.4 |
| | chain addition of (1→2)-linked Galp residues | |
| O3 | [ →2)-α-_{D}-Man-(1→2)-α-_{D}-Man-(1→3)- | Formula |
| | α-_{D}-Man-(1→3)-α-_{D}-Man-(1→ ]ₙ | K.O3 |
| O4 | [→4)-α-D-Galp-(1→2)-β-D-Ribf-(1→)]ₙ | Formula |
| | | K.O4 |
| O5 | [ →2)-α-_{D}-Man-(1→2)-α-_{D}-Man- | Formula |
| | (1→3)-β-_{D}-Man-(1→ ]ₙ | K.O5 |
| O7 | [→2-a-_{L}-Rhap-(1→2)-β-_{D}-Ribf-(1→3)- | Formula |
| | α-_{L}-Rhap-(1→3)-α-_{L}-Rhap-(1→]ₙ | K.O7 |
| O12 | [α-Rhap-(1 →3)-β-GlcpNAc]ₙ | Formula |
| | | K.O12 |
| O8 | [→3)-α-_{D}-Galp-(1→3)-β-_{D}-Galf-(1→]ₙ- | Formula |
| | nonstoichiometrically O-acetylated | K.O8 |

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1: E. coli and S. enterica Strains

Clinical strains and derivatives are listed in Table 3. Additional reference strains included: O25K5H1, a clinical O25a serotype strain; and S. enterica serovar Typhimurium strain LT2.

Gene knockouts in E. coli strains removing the targeted open-reading frame but leaving a short scar sequence were constructed.

The hydrolyzed O-antigen chain and core sugars are indicated subsequently as O-Polysaccharide (OPS) for simplicity.

TABLE 3
E. coli Strains

| | Strain | | |
|---|---|---|---|
| Strain | Alias | Genotype | Serotype |
| GAR2401 | PFEEC0100 | wt (blood isolate) | O25b |
| ‘2401ΔwzzB | — | ΔwzzB | O25b |
| ‘2401ΔAraAΔ(OPS) | — | ΔAraA Δ(rflB-wzzB) | OPS- |
| O25K5H1 | PFEEC0101 | wt | O25a |
| O25K5H1ΔwzzB | | ΔwzzB | O25a |
| BD559 | — | W3110 ΔAraA | OPS- |
| | | ΔfhuA ΔrecA | |
| BD559ΔwzzB | — | W3110ΔAraA ΔfhuA | OPS- |
| | | ΔrecAΔwzzB | |
| BD559Δ(OPS) | — | BD559 Δ(rflB-wzzB) | OPS- |
| GAR2831 | PFEEC0102 | wt (blood isolate) | O25b |
| GAR865 | PFEEC0103 | wt (blood isolate) | O2 |
| GAR868 | PFEEC0104 | wt (blood isolate) | O2 |
| GAR869 | PFEEC0105 | wt (blood isolate) | O15 |
| GAR872 | PFEEC0106 | wt (blood isolate) | O1 |
| GAR878 | PFEEC0107 | wt (blood isolate) | O75 |
| GAR896 | PFEEC0108 | wt (blood isolate) | O15 |
| GAR1902 | PFEEC0109 | wt (blood isolate) | O6 |
| Atlas 187913 | PFEEC0068 | wt (blood isolate) | O25b |
| Salmonella | — | wt | N/A |
| enterica serovar | | | |
| Typhimurium | | | |
| strain LT2 | | | |

### Example 2: Oligonucleotide Primers for WZZB, FEPE and O-Antigen Gene Cluster Cloning

TABLE 4
Oligonucleotide Primers

| Name | Primer Sequence | Comments |
|---|---|---|
| LT2wzzB_S | GAAGCAAACCGTACGCGTAAAG (SEQ ID NO: 1) | based on Genbank |
| | | GCA_000006945.2 |
| | | Salmonella enterica |
| LT2wzzB_AS | CGACCAGCTCTTACACGGCG (SEQ ID NO: 2) | serovar Typhimurium |
| | | strain LT2 |
| O25bFepE_S | GAAATAGGACCACTAATAAATACACAAATTAATA | Based on Genbank |
| | AC (SEQ ID NO: 3) | GCA_000285655.3 |
| | | O25b EC958 strain |
| | | ST131 assembly and |
| | | O25b GAR2401 WGS |
| | | data |
| O25bFepE_A | ATAATTGACGATCCGGTTGCC (SEQ ID NO: 4) | |
| wzzB P1_S | GCTATTTACGCCCTGATTGTCTTTTGT (SEQ ID | based on E. coli K-12 |
| | NO: 5) | strain sequence, |
| | | Genbank MG1655 |
| wzzB P2_AS | ATTGAGAACCTGCGTAAACGGC (SEQ ID NO: 6) | NC_000913.3 orW3110 |
| | | assembly |
| | | GCA_000010245.1 |
| wzzB P3_S | TGAAGAGCGGTTCAGATAACTTCC (SEQ ID NO: | |
| | 7) | |
| | (UDP-glucose-6-dehydrogenase) | |
| wzzB P4_AS | CGATCCGGAAACCTCCTACAC (SEQ ID NO: 8) | |
| | (PhosphorTbosyl-AMP cyclohydrolase/ | |
| | PhosphorTbosyl | |
| | -ATP pyrophosphohydrolase) | |
| 0157 FepE_S | GATTATTCGCGCAACGCTAAACAGAT (SEQ ID | E. coli O157 fepE |
| | NO: 9) | (based on Genbank |
| | | EDL933 strain |
| | | GCA 000732965.1) |
| 0157 | TGATCATTGACGATCCGGTAGCC (SEQ ID NO: | |
| FepE_AS | 10) | |
| pBAD33_ | CGGTAGCTGTAAAGCCAGGGGCGGTAGCGTG | Adaptor has central |
| adaptor_ | GTTTAAACCCAAGCAACAGATCGGCGTCGTCG | PmeI site and homology |
| S | GTATGGA (SEQ ID NO: 11) | to conserved 5′ OAg |
| | | operon promoter and 3′ |
| | | gnd gene sequences |
| pBAD33_ | AGCTTCCATACCGACGACGCCGATCTGTTGCTT | |
| adaptor_ | GGGTTTAAACCACGCTACCGCCCCTGGCTTTA | |
| AS | CAGCTACCGAGCT (SEQ ID NO: 12) | |
| JUMPSTART_ | GGTAGCTGTAAAGCCAGGGGCGGTAGCGTG | Universal Jumpstart |
| r | (SEQ ID NO: 13) | (OAg operon promoter) |
| gnd_f | CCATACCGACGACGCCGATCTGTTGCTTGG | Universal 3′ OAg (gnd) |
| | (SEQ ID NO: 14) | operon antisense primer |

### Example 3: Plasmids

Plasmid vectors and subclones are listed in Table 5. PCR fragments harboring various E. coli and Salmonella wzzB and fepE genes were amplified from purified genomic DNA and subcloned into the high copy number plasmid provided in the Invitrogen PCR@Blunt cloning kit. This plasmid is based on the pUC replicon. Primers P3 and P4 were used to amplify E. coli wzzB genes with their native promoter, and are designed to bind to regions in proximal and distal genes encoding UDP-glucose-6-dehydrogenase and phosphoribosyladenine nucleotide hydrolase respectively (annotated in Genbank MG1655 NC_000913.3). A PCR fragment containing Salmonella fepE gene and promoter were amplified using primers previously described. Analogous E. coli fepE primers were designed based on available Genbank genome sequences or whole genome data generated internally (in case of GAR2401 and O25K5H1). Low copy number plasmid pBAD33 was used to express O-antigen biosynthetic genes under control of the arabinose promoter. The plasmid was first modified to facilitate cloning (via Gibson method) of long PCR fragments amplified using universal primers homologous to the 5′ promoter and 3′ 6-phosphogluconate dehydrogenase (gnd) gene Table 5.

TABLE 5
Plasmids

| | | Resistance | |
|---|---|---|---|
| Name | Replicon | marker | Comments |
| PCR ®Blunt | pUC | KanR | Invitrogen PCR |
| II TOPO | | | cloning vector |
| pBAD33 | P15a | CamR | Arabinose inducible vector |
| pBAD33-OAg | P15a | CamR | OAg operon Gibson |
| | | | cloning vector |
| pBAD33-O25b | P15a | CamR | O25b OAg expression |
| | | | plasmid |
| pBAD33-O21 | P15a | CamR | O21 OAg expression |
| | | | plasmid |
| pBAD33-O16 | P15a | CamR | O16 OAg expression |
| | | | plasmid |
| pBAD33-O75 | P15a | CamR | O75 OAg expression |
| | | | plasmid |
| pBAD33-O1 | P15a | CamR | O1 OAg expression |
| | | | plasmid |
| PBAD33-O2 | P15a | CamR | O2 OAg expression |
| | | | plasmid |
| pTOPO-O25b | pUC | KanR | GAR 2401 gDNA |
| 2401 wzzB | | | template |
| pTOPO-O25b | pUC | KanR | |
| 2401 fepE | | | |
| PTOPO-K12 | pUC | KanR | E. coli K-12 strain |
| wzzB | | | gDNA template |
| pTOPO-O25a | pUC | KanR | E. coli O25a strain |
| wzzB | | | O25K5H1 |
| pTOPO-O25a | pUC | KanR | gDNA template |
| fepE | | | |
| pTOPO- | pUC | KanR | Salmonella enterica |
| Salmonella LT2 | | | serovar Typhimurium |
| wzzB | | | strain LT2 gDNA |
| pTOPO- | pUC | KanR | template |
| Salmonella LT2 | | | |
| fepE | | | |
| pTOPO-O25a | pUC | KanR | O25a ETEC strain gDNA |
| ETEC wzzB | | | purchased from |
| pTOPO-O25a | pUC | KanR | ATCC (“NR-5” |
| ETEC fepE | | | E2539-C1) |
| pTOPO- | pUC | KanR | O157:H7:K-Shigella |
| O157fepE | | | toxin strain gDNA |
| | | | purchased from ATCC |
| | | | (EDL933 #43895D-5) |

### Example 4: O-Antigen Purification

The fermentation broth was treated with acetic acid to a final concentration of 1-2% (final pH of 4.1). The extraction of OAg and delipidation were achieved by heating the acid treated broth to 100° C. for 2 hours. At the end of the acid hydrolysis, the batch was cooled to ambient temperature and 14% NH₄OH was added to a final pH of 6.1. The neutralized broth was centrifuged and the centrate was collected. To the centrate was added CaCl₂ in sodium phosphate and the resulting slurry was incubated for 30 mins at room temperature.

The solids were removed by centrifugation and the centrate was concentrated 12-fold using a 10 kDa membrane, followed by two diafiltrations against water. The retentate which contained OAg was then purified using a carbon filter. The carbon filtrate was diluted 1:1 (v/v) with 4.0M ammonium sulfate. The final ammonium sulfate concentration was 2M. The ammonium sulfate treated carbon filtrate was further purified using a membrane with 2M ammonium sulfate as the running buffer. The OAg was collected in the flow through. For the long OAg the HIC filtrate was concentrated and then buffer exchanged against water (20 diavolumes) using a 5 kDa membrane. For the short (native) OAg polysaccharide, the MWCO was further reduced to enhance yield.

In another embodiment, the solids were removed by centrifugation and the centrate was concentrated 12-fold using a 10 kDa membrane, followed by two diafiltrations against water or 20-25 mM Tris buffer that contained 20-25 mM NaCl pH 7.2-7. The retentate which contained OAg was then purified using a carbon filter. The carbon filtrate was further purified by ion exchange (IEX) membrane chromatography. The IEX filtrate was then diluted 1:1 (v/v) with 4.0M ammonium sulfate. The final ammonium sulfate concentration was 2M. The ammonium sulfate treated IEX filtrate was further purified using a HIC membrane with 2M ammonium sulfate as the running buffer. The OAg was collected in the flow through. The HIC filtrate was concentrated and then buffer exchanged against water (20 diavolumes) using a 5 kDa membrane.

Background of EXAMPLES 5-17: The examples illustrated here demonstrate a platform-based process for the purification of all serotypes of O-antigen polysaccharides (refer also to O-antigen or O—Ag), which may contain inner/outer oligosaccharides.

The purification process described here is applicable to both short chain and long chain O—Ag polysaccharides. Most examples given here are for long chain O—Ag, except for Example 10 and 11, which are short chain O—Ag for E. coli serotype O8 and O9, respectively.

### Example 5: Methods for Purifying E. coli O-Antigen Polysaccharides

### 1. Release of O-Antigen

The process begins with acid hydrolysis after the completion of fermentation to release the O—Ag from the lipopolysaccharides (LPS). This was achieved by treating the crude suspension of serotype O25b cell culture with the acetic acid to the final concentration of 1.0% (v/v) that brought the pH to about 4.0. The acidic broth was then heated to the temperature of 100° C. and incubated for 2.0 hours. After the product was released, the batch was cooled to the ambient temperature of 20-30° C.

To further refine the release conditions for O25b O—Ag, a design of experiment (DOE) was set up to examine the effect of pH, temperature, and hold time on % KDO, concentration, molecular weight (MW), and O-acetate. Factors examined in the DOE study are shown in Table 1-1. Note that the initial concentration, the high limit for KDO and the high limit for the O-acetate were set at 3.5 mg/mL, 2% and 1.0 mM, respectively, for this study.

TABLE 1-1
DOE Factors Examined in O25b Acid Hydrolysis Study

| Factor | Range |
|---|---|
| pH | 3.0-5.0 |
| Temperature (° C.) | 80-100 |
| Hold time (hour) | 1.0-4.0 |

The predictional responses of concentration, % KDO and O-acerate at time points of 1.0, 2.0, 2.5 and 4.0 hours, respectively, were assessed. The molecular weight at all conditions was flat, around 50-54 kDa, except at the conditions for temperature of 80° C. where the MW was large. This was perhaps due to the incompleted release of the product, in which case the O—Ag might be associated with the cell components or other non-specific surface polysaccharides. Therefore, the DOE model could not provide the predictive response for the MW at these conditions. At 4.0-hour time point, there was no % KDO within the range.

Based on this DOE result, the conditions for acid hydrolysis are reset at pH 3.8±0.1, temperature 95±5° C. and hold time of 2.0 hours. These release conditions were used for all other serotypes of O—Ag polysaccharides in the subsequent examples.

### 2. Flocculation

The main purpose of this step is to precipitate cell debris, host cell proteins and nucleic acids from the broth that contains the released product. It also enhances the efficiency of the downstream clarification unit operation. The acidified broth after the release of product from Step 1 was treated with the 10% Alum solution to the final concentration of 2% (w/v), and the pH was further adjusted to 3.2 using sulfuric acid. The flocculated slurry was incubated at ambient temperature for 1.0 hour, followed by centrifugation at 12,000-14,000 g for 30 minutes. The supernatant was then filtered by a 0.2-μm filter or another suitable depth filter to remove any small particles that might skipped into the solution. The depth filtrate was proceeding to the initial purification of UFDF-1.

Alternatively, the acidified broth was neutralized to pH of 6.0-7.0. The neutralized filtrate was centrifuged at 12,000 g for 30 minutes. The neutralized supernatant was filtered via 0.2-μm filter. The neutralized filtrate can be stored at 4° C. for at least one week without any adverse impact on the quality of the product. When the batch is ready for purification, the neutralized filtrate will go through the flocculation process described in the above paragraph. The subsequent purification steps illustrated in this example use this flocculation method, unless indicated otherwise.

A comparison of the SEC-HPLC chromatographic profiles for the neutralized filtrate that contained the product and depth filtrate after flocculation was conducted. From both the refractive index (RI) and UV 280 detection results, the flocculation step removed a substantial amount of impurities inherited from the fermentation media.

### 3. Ultrafiltration/Diafiltration-(UFDF-1)

The depth filtrate from Step 2 above is further purified through ultrafiltration and diafiltration (UFDF) using 10-kDa Sartocon Hydrosart membrane. The amount of depth filtrate processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step was 20 mM citrate/0.1 M NaCl pH 6.0 followed by the second buffer of 20 mM Tris/20 mM NaCl pH 7.2. The numbers of diavolumes were 10 for both diafiltration steps. The retentate from the UFDF was collected and analyzed. The conductivity and UV profiles during the UFDF run indicate that a majority of the small MW as well as UV related impurities were removed during the first diafiltration, evidenced by the significant drop on the UV signals for the permeate. A comparison of SEC-HPLC chromatograms of the depth filtrate and the retentate of UFDF-1, for both RI and UV detections was analyzed.

### 4. Carbon Filtration

This unit operation reduces the level of host cell impurities such as proteins and nucleic acids as well as colored impurities (see WO2008118752). The 3M R32SP carbon filter is used at loading of approximately 150 g of O—Ag from retentate of UFDF-1 per m² of carbon filter area. The carbon filter was first rinsed with water followed by the diafiltration buffer at approximately 20 liters of buffer per m² of filter area. The retentate from UFDF-1 was then filtered at a flow rate of 50 LMH (liters per m² per hour) in a single pass mode. The filter was then rinsed with the buffer, and the filtrate including rinse that contained the product was collected as carbon filtrate.

SEC-HPLC chromatograms for the UFDF retentate and the carbon filtrate indicate that the RI and UV280 related impurities were removed and carbon filtrate became visually colorless.

### 5. IEX Membrane Chromatography

This step was originally developed for serotypes O2 and O6 O-antigens to remove the non-specific negatively charged impurities (see Example 6 and 15). Therefore, by exploring the electrostatic interaction property of these molecules using the ion exchange (IEX) membrane chromatography, impurities from non-serotype specific extrar or intracellular polysaccharides may be removed.

The IEX membrane used here is Millipure's NatriFlo membrane cassette. Alternatively, the Sartobind Q membrane from Sartorius Stedim can also be used. All examples illustrated here used the NatriFlo membrane (or refer to thereafter as HD-Q) for the IEX membrane chromatography, unless indicated otherwise.

The membrane was first equilibrated with the 20 mM Tris/20 mM NaCl pH 7.2, typically 20-30 membrane volume (MV). The carbon filtrate from previous step was pumped through the membrane at flow rate of 30-40 mL/min with about 200-250 mg of O—Ag in carbon filtrate per mL of MV. The flow through effluent or filtrate that contained the product was collected. The membrane was rinsed with equilibration buffer and then washed with the high salt buffer of 20 mM Tris/1.0M NaCl pH 7.2. The conductivity and UV280 profiles of the IEX membrane chromatographic run In this profile, the UV signal showed a peak eluted out during the high salt wash, indicating there was an unknown negatively charged impurity that was present in the carbon filtrate.

The SEC-HPLC chromatograms for the carbon filtrate, IEX filtrate and the high salt wash effluent indicate that the high salt wash chromatogram showed a small peak at the same retention time as the product peak. This suggests that this unknown substance has a stronger ionic strength than the O25b O—Ag.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The carbon filtrate from Step 4 was treated with 4.0M ammonium sulfate (AS) solution to the final concentration of 2.0M. The phenyl membrane was first equilibrated with the running buffer of 2.0M ammonium sulfate (AS). The AS treated carbon filtrate was pushed through the HIC membrane at flow rate of 40-mL/min. The HIC membrane was then rinse with the running buffer, followed by the water wash. The flow through effluent along with the buffer rinse was collected as HIC filtrate, and the water wash was also collected for analysis. Alternatively this HIC filtration step can also be performed for IEX filtrate from Step 4.

The AKTA Avant chromatography run for the HIC purification was analyzed. The product was in the flow through effluent, and the peak shown in the water wash was non-specified hydrophobic related impurity that bound onto the HIC membrane. SEC-HPLC chromatograms for the carbon filtrate and HIC filtrateindicate that small front shoulder impurity peak that is present in the carbon filtrate was removed by the HIC filtration step.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The conductivity and the UV280 signals of the permeate as a function of DV during the diafiltration were analyzed. After 10 DVs, the conductivity reached steady state, indicating the completion of buffer exchange.

The comparison of SEC-HPLC chromatograms for HIC filtrate and final purified O25b O—Ag after the UFDF-2 was analyzed. Table 1-2 below summarizes the quality attributes of the final purified O25b O—Ag.

TABLE 1-2
Summary of Quality Attributes of Purified O25b O-Ag

| | |
|---|---|
| Purity by SEC-HPLC | >99.9% |
| Molecular weight (kDa) | 44.0 |
| Residual Protein (%) | 0.21 |
| Residual Nucleic Acid (%) | 0.05 |
| Endotoxin (EU/mg) | 0.1 |
| NMR structural identification | Conforms |

### Example 6. Purification of E. coli O-Antigen Serotype 06

### 1. Release of O-Antigen

The process begins with acid hydrolysis after the completion of the fermentation process to release the O—Ag from the lipopolysaccharides (LPS). Based on the DOE studies conducted for the O25b (see Example 5), the conditions used for the acid hydrolysis are pH 3.8±0.1, temperature 95±5° C. and hold time of 2.0 hours. The acetic acid was used, and this step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

The comparison of the SEC-HPLC chromatographic profiles for the acidified broth that contained the product and depth filtrate after flocculation was analyzed. Both the refractive index (RI) and UV 280 detection indicate that the flocculation step removed a substantial amount of impurities inherited from the fermentation media.

### 3. Ultrafiltration/Diafiltration-(UFDF-1)

The depth filtrate from Step 2 above is further purified through ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 20 and 10 for each diafiltration step, respectively. The comparison of SEC-HPLC chromatograms for the depth filtrate and the retentate of UFDF-1 indicate that a majority of the small MW as well as UV related impurities were removed during the diafiltration. However, there was a big front shoulder impurity peak associated with the product peak in the RI chromatogram.

### 4. Carbon Filtration

This unit operation reduces the level of host cell impurities such as proteins and nucleic acids as well as colored impurities (see WO2008118752). The 3M R32SP carbon filter is used at loading of approximately 150 g of O—Ag per m² of carbon filter area. The carbon filter was first rinsed with water followed by the diafiltration buffer at approximately 20 liters of buffer per m² of membrane area. The retentate from UFDF-1 was then filtered at a flow rate of 50 LMH (liters per m² per hour) in a single pass mode. The filter was then rinsed with the buffer, and the filtrate/rinse that contained the product was collected. the SEC-HPLC chromatograms for the UFDF retentate and the carbon filtrateindicate that the RI and UV280 related impurities were removed and carbon filtrate literately became colorless. However, the front shoulder impurity peak is still present in the carbon filtrate.

### 5. IEX Membrane Chromatography

This step was developed to remove the non-specific negatively charged impurity (see Section IEX membrane Chromatography in Example 5). The HD-Q membrane was first equilibrated with the 20 mM Tris/20 mM NaCl pH 7.2, typically 20-30 membrane volume (MV). The carbon filtrate was then loaded onto the membrane at about 100-250 mg of O—Ag per mL of MV. The flow through effluent or filtrate that contained the product was collected. The membrane was rinsed with equilibration buffer and then washed with the high salt buffer, 20 mM Tris/1.0M NaCl pH 7.2.

The SEC-HPLC chromatograms for the carbon filtrate, and IEX filtrateindicate that the big front shoulder peak associated with the product peak was removed from the carbon filtrate.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate from previous step was treated with 4.0M ammonium sulfate (AS) solution to the final concentration of 2.0M. The phenyl membrane was first equilibrated with the running buffer of 2.0M ammonium sulfate. The AS treated IEX filtrate was filtered through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis.

The SEC-HPLC chromatograms for the HIC filtrate and HIC filtrateindicate that small front shoulder impurity peak that is present in the carbon filtrate was removed by the HIC filtration step.

### 7. Ultrafiltration/Diafiltration-(UFDF-2)

This unit operation concentrates the product to the desired concentration and replaces the ammonium sulfate from the previous HIC purification step with the desirable buffer or water for conjugation. This step is performed using a 5-kDa molecular weight cutoff membrane of Sartocon Hydrosart from Sartorius.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively.

The comparison of SEC-HPLC chromatograms for HIC filtrate and final purified O6 O—Ag after the UFDF-2 was analyzed. Table-2-1 below summarizes the quality attributes of the final purified O6 O—Ag.

TABLE 2-1
Summary of Quality Attributes of Purified O6 O-Ag

| | |
|---|---|
| Purity by SEC-HPLC | >99.9% |
| Molecular weight (kDa) | 45.5 |
| Residual Protein (%) | 0.2 |
| Residual Nucleic Acid (%) | 0.03 |
| Endotoxin (EU/mg) | 0.07 |
| NMR structural identification | Conforms |

### Example 7. Purification of E. coli O-Antigen Serotype O75

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1M NaCl pH 6.0 followed by the second buffer of 20 mM Tris/20 mM NaCl pH 7.2. The numbers of diavolumes are 10 and 15 for each diafiltration step, respectively. The SEC-HPLC chromatograms of neutralized filtrate, depth filtrate after flocculation and retentate after the UFDF-1 were analyzed. The effectiveness of flocculation and UFDF-1 for removing the host cell proteins and small MW impurities was demonstrated by both RI and UV280 chromatograms.

### 4. Carbon Filtration

This unit operation reduces the level of host cell impurities such as proteins and nucleic acids as well as colored impurities (see WO2008118752). The 3M R32SP carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse indicate that the substantial amount of the UV related small MW impurities were removed.

### 5. IEX Membrane Chromatography

This step was developed to remove the non-specific negatively charged impurity (see Section IEX membrane Chromatography in Example 5). The IEX membrane was first equilibrated with the 20 mM Tris/20 mM NaCl pH 7.2, typically 20-30 membrane volume (MV). The carbon filtrate was then loaded onto the membrane at about 200-250 mg of O—Ag per mL of MV. The flow through effluent or filtrate that contained the product was collected. The membrane was rinsed with equilibration buffer and then washed with the high salt buffer, 20 mM Tris/1.0M NaCl pH 7.2.

The conductivity and UV profiles of the IEX membrane chromatographic run were analyzed. In this profile, the UV signal showed a peak in the high salt wash, indicating there was an unknown negatively charged impurity that was present in the carbon filtrate.

The SEC-HPLC chromatograms for the carbon filtrate, IEX filtrate and the high salt wash effluent indicate that the high salt elution sample showed a double peak underneath the product peak

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis.

SEC-HPLC chromatograms for the IEX filtrate, HIC filtrate, HIC water wash and purified O75 O—Ag indicate that the water wash sample also showed a peak that eluted at the same retention time as the product in the SEC-HPLC, indicating that small amount of unknown substance that had higher hydrophobicity probably present in the IEX filtrate.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-1 was collected along with the rinse. The final pool was filtered through a 0.2-μm filter. The SEC-HPLC profile for the final purified O75 O—Ag was analyzed. Table-3-1 below summarizes the quality attributes of the final purified O75 O—Ag.

TABLE 3-1
Summary of Quality Attributes of Purified O75 O-Ag

| | |
|---|---|
| Purity by SEC-HPLC | >99.0% |
| Molecular weight (kDa) | 53.4 |
| Residual Protein (%) | 0.30 |
| Residual Nucleic Acid (%) | 0.04 |
| Endotoxin (EU/mg) | 0.12 |
| NMR structural identification | Conforms |

### Example 8. Purification of E. coli O-Antigen Serotype O1

### 1. Release of O-Antigen

The process begins with acid hydrolysis after fermentation to release the O—Ag from the lipopolysaccharides (LPS). Based on the DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

The comparison of SEC-HPLC chromatograms for the neutralized filtrate and depth filtrate after flocculation was analyzed. This data indicate that substantial amount of impurities were removed by flocculation step.

### 3. Ultrafiltration/Diafiltration-(UFDF-1)

Purification begins with the depth filtrate (from step 2 above) by ultrafiltration and diafiltration using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 10 and 15 for each diafiltration step, respectively. The SEC-HPLC chromatograms of depth filtrate and retentate after the UFDF-1 were analyzed. The effectiveness of flocculation and UFDF-1 for removing the host cell proteins and small MW impurities was demonstrated by both RI and UV280 chromatograms.

### 4. Carbon Filtration

This unit operation reduces the level of host cell impurities such as proteins and nucleic acids as well as colored impurities (see WO2008118752). The 3M carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in a single pass mode. Following the filtration, the carbon filter was rinsed with the buffer. The filtrate and rinse that contained the product were combined as carbon filtrate.

The SEC-HPLC chromatograms for the UFDF retentate and the carbon filtrate and carbon bulk, which included rinse indicate that RI chromatographic profile did not change much but the substantial amount of the UV and color related impurity was removed.

### 5. Hydrophobic Interaction Chromatography (HIC)

This unit operation removes any impurities that had hydrophobic characteristics, such as residual lipid A left from the acid hydrolysis step and assure that endotoxin level will be kept at minimum level. The Sartobind Phenyl 150-mL membrane was used for the HIC step. The carbon filtrate was treated with 4.0M ammonium sulfate (AS) solution to the final concentration of 2.0M. The phenyl membrane was first equilibrated with the running buffer of 2.0M ammonium sulfate. The AS treated carbon filtrate was filtered through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. SEC-HPLC chromatograms for the carbon filtrate, and HIC filtrateindicate that there was no detectable improvement based on the SEC-HPLC profiles, but the endotoxin level was significantly reduced (see Table 4-1 below).

### 6. Ultrafiltration/Diafiltration-(UFDF-2)

This unit operation concentrates the product to the desired concentration and replaces the ammonium sulfate with the desirable buffer or water for conjugation. This step is performed using a Sartocon Hydrosart 5-kDa membrane from Sartorius.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-1 was collected along with the rinse. The final pool was filtered through a 0.2-μm filter. The SEC-HPLC profile for the final purified O1 O—Ag was analyzed. Table 4-1 below summarizes the quality attributes of the final purified O1 O—Ag.

TABLE 4-1
Summary of Quality Attributes of Purified O1 O-Ag

| | |
|---|---|
| Purity by SEC-HPLC | >99.9% |
| Molecular weight (kDa) | 38.0 |
| Residual Protein (%) | 0.33 |
| Residual Nucleic Acid (%) | 0.06 |
| Endotoxin (EU/mg) | 0.05 |
| NMR structural identification | Conforms |

### Example 9. Purification of E. coli O-Antigen Serotype 15

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here with the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1. The comparison of SEC-HPLC chromatograms for the neutralized filtrate and depth filtrate after flocculation were analyzed.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 10 for each diafiltration step, respectively. The actual experiment UFDF run indicate that a majority of the small MW as well as UV related impurities were removed during the diafiltration, and this was also evidenced in the SEC-HPLC chromatograms of retentate after the UFDF-1. The effectiveness of flocculation and UFDF-1 for removing the host cell proteins and small MW impurities was demonstrated by both RI and UV280 chromatograms.

### 4. Carbon Filtration

This unit operation reduces the level of host cell impurities such as proteins and nucleic acids as well as colored impurities (see WO2008118752). The 3M carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse, indicate that the substantial amount of the small MW impurities were removed.

### 5. IEX Membrane Chromatography

This step was developed to remove the non-specific negatively charged impurity (see Section IEX membrane Chromatography in Example 5). The IEX membrane used here is Millipure's NatriFlo cassette. Alternatively, the Sartobind Q membrane from Sartorius Stedim can also be used. The membrane was first equilibrated with the 25 mM Tris/25 mM NaCl pH 7.5, typically 20-30 membrane volume (MV). The carbon filtrate was then loaded onto the membrane at about 200-250 mg of O—Ag per mL of MV. The flow through effluent or filtrate that contained the product was collected. The membrane was rinsed with equilibration buffer and then washed with the high salt buffer, 25 mM Tris/1.0M NaCl pH 7.5.

The conductivity and UV profiles of the IEX membrane chromatographic run were analyzed. In this profile, the UV signal showed a peak during the high salt wash, indicating there was an unknown negatively charged impurity that was present in the carbon filtrate. The SEC-HPLC chromatograms for the carbon filtrate, IEX filtrate and the high salt wash effluent indicate that the high salt elution sample showed a small double peak with the similar retention time of the product.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. The chromatographic profiles of UV and conductivity for the HIC membrane filtration were analyzed. There was a small visible peak shown in the water wash step, indicating a trace amount of the unspecified substance bound onto the HIC membrane.

SEC-HPLC chromatograms for the IEX filtrate, HIC filtrate, HIC water wash and purified O15 O—Ag indicate that the water wash sample showed a double peak that eluted slightly before the product in the SEC-HPLC, indicating that some unknown substance present in the HD-Q stream.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-2 was collected along with the rinse. The final pool was filtered through a 0.2-μm filter. The conductivity and the UV280 signals of the permeate as a function of DV during the diafiltration indicate that after 10 DV, the conductivity reached steady state, indicating the completion of buffer exchange. The SEC-HPLC profiles for the final purified O15 O—Ag were analyzed. Table 5-1 below summarizes the quality attributes of the final purified O15 O—Ag.

TABLE 5-1
Summary of Quality Attributes of Purified O15 O-Ag

| | |
|---|---|
| Purity by SEC-HPLC | >99.9% |
| Molecular weight (kDa) | 56.0 |
| Residual Protein (%) | 0.0023 |
| Residual Nucleic Acid (%) | <0.001 |
| Endotoxin (EU/mg) | <0.04 |
| NMR structural identification | Conforms |

### Example 10. Purification of E. coli O-Antigen Serotype O8

### 1. Release of O-Antigen

The serotype O8 is a short chain O-antigen, and the molecular weight is expected to be in the range of 10-15 kDa. However, the purification process outlined also applies to all the short chain E. coli O—Ag. The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here with the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 10 for each diafiltration step, respectively. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the carbon filtrate were analyzed. The fact that the product peak was reduced after the carbon filtration, indicating the non-specific adsorption mode by which carbon filter was designed for. Nevertheless, the color related impurities were mostly removed.

### 5. IEX Membrane Chromatography

This step was developed to remove the non-specific negatively charged impurity (see Section IEX membrane Chromatography in Example 5). The IEX membrane used here is Millipure's NatriFlo (HD-Q) cassette. Alternatively, the Sartobind Q membrane from Sartorius Stedim can also be used. The membrane was first equilibrated with the 20 mM Tris/20 mM NaCl pH 7.2, typically 20-30 membrane volume (MV). The carbon filtrate was then loaded onto the membrane at about 200-250 g of O—Ag per mL of MV. The flow through effluent or filtrate that contained the product was collected. The membrane was rinsed with equilibration buffer and then washed with the high salt buffer, 20 mM Tris/1.0M NaCl pH 7.2.

The conductivity and UV profiles of the IEX membrane chromatographic run was analyzed. In this profile, the UV signal showed a peak during the high salt wash, indicating there was an unknown negatively charged impurity that was present in the carbon filtrate.

The SEC-HPLC chromatograms for the carbon filtrate, IEX filtrate and the high salt wash effluent indicate that the high salt elution sample showed a small double peak with the similar retention time of the product.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. The chromatographic profiles of UV and conductivity for the HIC membrane filtration were analyzed. There was a visible peak shown in the water wash step, indicating a trace amount of the unspecified hydrophobic substance bound onto the HIC membrane.

SEC-HPLC chromatograms for the IEX filtrate, HIC filtrate, HIC water wash and purified O8 O—Ag indicate that the water wash sample showed a double peak that eluted slightly before the product in the SEC-HPLC, indicating that only very small amount of unknown substance present in the HD-Q stream.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 200 LMH and 0.5 bars, respectively. The retentate from the UFDF-2 was collected along with the rinse. The final pool was filtered through a 0.2-μm filter. The SEC-HPLC profiles for the final purified O8 O—Ag were analyzed. Table 6-1 below summarizes the quality attributes of the final purified O8 O—Ag.

TABLE 6-1
Summary of Quality Attributes of Purified O8 O-Ag

| | |
|---|---|
| Purity by SEC-HPLC | >99.9% |
| Molecular weight (kDa) | 11.5 |
| Residual Protein (%) | 0.08 |
| Residual Nucleic Acid (%) | 0.02 |
| Endotoxin (EU/mg) | 0.96 |
| NMR structural identification | Conforms |

### Example 11. Purification of E. coli O-Antigen Serotype O9

### 1. Release of O-Antigen

The serotype O9 is also a short chain O-antigen, and the molecular weight is expected to be in the range of 10-15 kDa. The purification process outlined was used. The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1. The comparison of SEC-HPLC chromatographic profiles for the neutralized filtrate and the depth filtrate was analyzed.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 10 for each diafiltration step, respectively. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the carbon filtrate were analyzed. The fact that the product peak was reduced after the carbon filtration, indicating the non-specific adsorption mode by which carbon filter was designed for. Nevertheless, the color related impurities were mostly removed.

### 5. IEX Membrane Chromatography

This step was developed to remove the non-specific negatively charged impurity (see Section IEX membrane Chromatography in Example 5). The IEX membrane used here is Millipure's NatriFlo (HD-Q) cassette. Alternatively, the Sartobind Q membrane from Sartorius Stedim can also be used. The membrane was first equilibrated with the 20 mM Tris/20 mM NaCl pH 7.2, typically 20-30 membrane volume (MV). The carbon filtrate was then loaded onto the membrane at about 200-250 mg of O—Ag per mL of MV. The flow through effluent or filtrate that contained the product was collected. The membrane was rinsed with equilibration buffer and then washed with the high salt buffer, 20 mM Tris/1.0M NaCl pH 7.2.

The conductivity and UV profiles of the IEX membrane chromatographic run was analyzed. In this profile, the UV signal showed a peak during the high salt wash, indicating there was an unknown negatively charged impurity that was present in the carbon filtrate. The SEC-HPLC chromatograms for the carbon filtrate, IEX filtrate and the high salt wash effluent indicate that the high salt elution sample showed a small peak with the similar retention time of the product.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. The chromatographic profiles of UV and conductivity for the HIC membrane filtration.

SEC-HPLC chromatograms for the IEX filtrate, HIC filtrate, HIC water wash and purified O8 O—Ag indicate that the water wash sample showed no visible peak in the RI detection, indicating that there was no hydrophobic related substance present in the IEX stream.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 200 LMH and 0.5 bars, respectively. The retentate from the UFDF-2 was collected along with the rinse. The final pool was filtered through a 0.2-μm filter. The SEC-HPLC profiles for the final purified O9 O—Ag were analyzed. Table 7-1 below summarizes the quality attributes of the final purified O9 O—Ag.

TABLE 7-1
Summary of Quality Attributes of Purified O9 O-Ag

| | |
|---|---|
| Purity by SEC-HPLC | >99.9% |
| Molecular weight (kDa) | 11.0 |
| Residual Protein (%) | 0.08 |
| Residual Nucleic Acid (%) | 0.15 |
| Endotoxin (EU/mg) | 0.1 |
| NMR structural identification | Conforms |

### Example 12. Purification of E. coli O-Antigen Serotype O21

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 25 mM Tris/25 mM NaCl pH 7.5. The numbers of diavolumes are 18 for each diafiltration step, respectively. The actual experiment UFDF run indicate that a majority of the small MW as well as UV related impurities were removed during the diafiltration. The SEC-HPLC chromatograms of retentate after the UFDF-1 were analyzed. The effectiveness of flocculation and UFDF-1 for removing the host cell proteins and small MW impurities was demonstrated by both RI and UV280 chromatograms.

### 4. Carbon Filtration

This unit operation reduces the level of host cell impurities such as proteins and nucleic acids as well as colored impurities (see WO2008118752). The 3M R55SP carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse indicate that the substantial amount of the UV related small MW impurities were removed.

### 5. IEX Membrane Chromatography

This step was developed to remove the non-specific negatively charged impurity (see Section IEX membrane Chromatography in Example 5). The IEX membrane used here is Millipure's NatriFlo cassette. Alternatively, the Sartobind Q membrane from Sartorius Stedim can also be used. The membrane was first equilibrated with the 25 mM Tris/25 mM NaCl pH 7.5, typically 20-30 membrane volume (MV). The carbon filtrate was then loaded onto the membrane at about 150-200 mg of O—Ag per mL of MV. The flow through effluent or filtrate that contained the product was collected. The membrane was rinsed with equilibration buffer and two step washings, the first one with 25 mM Tris/25 mM NaCl pH7.5 buffer and the second one with the high salt buffer, 25 mM Tris/1.0M NaCl pH 7.5.

The conductivity and UV profiles of the IEX membrane chromatographic run indicate that there were some unknown negatively charged impurities bound onto the membrane and they were subsequently removed during the step elutions.

The SEC-HPLC chromatograms for the carbon filtrate, IEX filtrate 1^{st} and 2^{nd} step wash eluates, respectively indicate that the two eluates from the step wash samples showed different profiles (in both RI and UV chromatograms) than that of the product.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. The SEC-HPLC chromatogram for the HIC filtrate were analyzed.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-2 was collected along with the rinse. The final pool was filtered through a 0.2-μm filter. The conductivity and the UV280 signals of the permeate as a function of DV during the diafiltration indicate that after 10 DV, the conductivity reached steady state, indicating the completion of buffer exchange. The SEC-HPLC profiles for the final purified O21 O—Ag were analyzed. Table 8-1 below summarizes the quality attributes of the final purified O21 O—Ag.

TABLE 8-1
Summary of Quality Attributes of Purified O21 O-Ag

| | |
|---|---|
| Purity by SEC-HPLC | >99.9% |
| Molecular weight (kDa) | 37.8 |
| Residual Protein (%) | 0.36 |
| Residual Nucleic Acid (%) | 0.16 |
| Endotoxin (EU/mg) | <0.13 |
| NMR structural identification | Conforms |

### Example 13. Purification of E. coli O-Antigen Serotype O4

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The main purpose of this step is to precipitate cell debris, host cell proteins and nucleic acids from the broth that contains the product. It also enhances the efficiency of the downstream clarification process. The flocculation was performed here for the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1. The SEC-HPLC chromatograms of neutralized filtrate and the depth filtrate were analyzed.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 25 mM Tris/25 mM NaCl pH 7.5. The numbers of diavolumes are 18 for each diafiltration step, respectively. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M R55SP carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse indicate that the substantial amount of the UV related small MW impurities were removed.

### 5. IEX Membrane Chromatography

This step was developed to remove the non-specific negatively charged impurity (see Section IEX membrane Chromatography in Example 5). The IEX membrane used here is Millipure's NatriFlo (HD-Q) cassette. Alternatively, the Sartobind Q membrane from Sartorius Stedim can also be used. The membrane was first equilibrated with the 20 mM Tris/20 mM NaCl pH 7.2, typically 20-30 membrane volume (MV). The carbon filtrate was then loaded onto the membrane at about 150-200 mg of O—Ag per mL of MV. The flow through effluent or filtrate that contained the product was collected. The membrane was rinsed with equilibration buffer and two step washings, the first one with 20 mM Tris/25 mM NaCl pH7.5 buffer and the second one with the high salt buffer, 25 mM Tris/1.0M NaCl pH 7.5. The SEC-HPLC chromatograms for the carbon filtrate and the IEX filtrate were analyzed.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent was collected along with the rinse as HIC filtrate, and the water wash was also collected for analysis.

The conductivity and UV profiles for the HIC membrane chromatography run indicate that the hydrophobic related impurities were bound onto the HIC membrane and subsequently washed out by the water. The SEC-HPLC chromatogram for the HIC filtrate and HIC wash was analyzed.

### 7. Ultrafiltration/Diafiltration-(UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-2 was collected along with the rinse. The final pool was filtered through a 0.2-μm filter. The SEC-HPLC profiles for the final purified O4 O—Ag were analyzed. Table 9-1 below summarizes the quality attributes of the final purified O4 O—Ag.

TABLE 9-1
Summary of Quality Attributes of Purified O4 O-Ag

| | |
|---|---|
| Purity by SEC-HPLC | >99.9% |
| Molecular weight (kDa) | 52.6 |
| Residual Protein (%) | 0.17 |
| Residual Nucleic Acid (%) | 0.04 |
| Endotoxin (EU/mg) | <0.19 |
| NMR structural identification | Conforms |

### Example 14. Purification of E. coli O-Antigen Serotype O2

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The flocculation was performed from the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 25 mM Tris/25 mM NaCl pH 7.5. The numbers of diavolumes are 18 for each diafiltration step, respectively. 10-1 shows the UV and conductivity profiles for the UFDF-1, as one can see that most UV related small molecular weight impurities were removed during the first diafiltration. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M carbon filter is used at loading of approximately 75-125 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse indicate that carbon filtration was very effective in removing the residual color and small MW impurities.

### 5. IEX Membrane Chromatography

This step was developed to remove the non-specific negatively charged impurity (see Section IEX membrane Chromatography in Example 5). The IEX membrane used here is Millipure's NatriFlo cassette. Alternatively, the Sartobind Q membrane from Sartorius Stedim can also be used. The membrane was first equilibrated with the 20 mM Tris/20 mM NaCl pH 7.2, typically 20-30 membrane volume (MV). The carbon filtrate was then loaded onto the membrane at about 50-100 mg of O—Ag per mL of MV. The flow through effluent or filtrate that contained the product was collected. The membrane was rinsed with equilibration buffer and two step washings, the first one with 25 mM Tris/25 mM NaCl pH7.5 buffer and the second one with the high salt buffer, 25 mM Tris/1.0M NaCl pH 7.5. The UV and conductivity profiles for the IEX membrane chromatography were analyzed. As it delineared that there was a peak eluted out in the high salt wash cycle, indicating that there was small amount of impurities that were bound onto the membrane. The SEC-HPLC chromatograms for the carbon filtrate and IEX filtrate were analyzed.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 1.2M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent was collected along with the rinse as HIC filtrate, and the water wash was also collected for analysis. The SEC-HPLC chromatogram for the HIC filtrate is was analyzed.

### 7. Ultrafiltration/Diafiltration-(UFDF-2)

This unit operation concentrates the product to the desired concentration and replaces the ammonium sulfate with water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-2 was collected along with the rinse. The final pool was filtered through a 0.2-μm filter. The SEC-HPLC profiles for the final purified O2 O—Ag were analyzed. Table 10-1 below summarizes the quality attributes of the final purified O2 O—Ag.

TABLE 10-1
Summary of Quality Attributes of Purified O2 O-Ag

| | |
|---|---|
| Purity by SEC-HPLC | >99.9% |
| Molecular weight (kDa) | 46.1 |
| Residual Protein (%) | 0.23 |
| Residual Nucleic Acid (%) | 0.05 |
| Endotoxin (EU/mg) | 3.2 |
| NMR structural identification | Conforms |

### Example 15. Purification of E. coli O-Antigen Serotype O11

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The flocculation was performed from the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 25 mM Tris/25 mM NaCl pH 7.5. The numbers of diavolumes are 18 for each diafiltration step, respectively. The UV and conductivity profiles for the UFDF-1 indicate that most UV related small molecular weight impurities were removed during the first diafiltration. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M R55SP carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected. The SEC-HPLC chromatograms for the UFDF retentate and the carbon filtrate and carbon bulk, which included rinse indicate that carbon filtration was very effective in removing the residual color and small MW impurities.

### 5. IEX Membrane Chromatography

This step was developed to remove the non-specific negatively charged impurity (see Section IEX membrane Chromatography in Example 5). The IEX membrane used here is Millipure's NatriFlo cassette. Alternatively, the Sartobind Q membrane from Sartorius Stedim can also be used. The membrane was first equilibrated with the 25 mM Tris/25 mM NaCl pH 7.5, typically 20-30 membrane volume (MV). The carbon filtrate was then loaded onto the membrane at about 100-125 mg of O—Ag per mL of MV. The flow through effluent or filtrate that contained the product was collected. The membrane was rinsed with equilibration buffer and two step washings, the first one with 25 mM Tris/25 mM NaCl pH7.5 buffer and the second one with the high salt buffer, 25 mM Tris/1.0M NaCl pH 7.5. The UV and conductivity profiles for the IEX membrane chromatography were analyzed. As it delineared that there was a peak eluted out in the high salt wash step, indicating that there was unknown impurity bound onto the membrane. The SEC-HPLC chromatograms for the carbon filtrate, IEX filtrate and 2 high salt wash samples were analyzed.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent was collected along with the rinse as HIC filtrate, and the water wash was also collected for analysis. The SEC-HPLC chromatogram for the HIC filtrate was analyzed.

### 7. Ultrafiltration/Diafiltration-(UFDF-2)

This unit operation concentrates the product to the desired concentration and replaces the ammonium sulfate with water for conjugation. This step is performed using a 5-kDa molecular weight cutoff filter.

The HIC filtrate was concentrated ˜10-folds and followed by diafiltration into water with ˜20 numbers of diavolumes (DV). The retentate from the UFDF-2 was collected along with the rinse. The final pool was filtered through a 0.2-μm filter. The SEC-HPLC profiles for the final purified O11 O—Ag were analyzed. Table 11-1 below summarizes the quality attributes of the final purified O11 O—Ag.

TABLE 11-1
Summary of Quality Attributes of Purified O11 O-Ag

| | |
|---|---|
| Purity by SEC-HPLC | >99.9% |
| Molecular weight (kDa) | 40.2 |
| Residual Protein (%) | 0.35 |
| Residual Nucleic Acid (%) | 0.14 |
| Endotoxin (EU/mg) | 0.36 |
| NMR structural identification | Conforms |

### Example 16. Purification of E. coli O-Antigen Serotype O18

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Flocculation

The flocculation was performed from the neutralized filtrate after the acid hydrolysis described in the Example 5 under Section of “Flocculation”. The 10% Alum solution was added to the neutralized filtrate to the final concentration of 2% (w/v), and the pH was further adjusted to 3.2 using sulfuric acid. The flocculated slurry is incubated at ambient temperature for 1.0 hour, followed by centrifugation at 12,000-14,000 g for 30 minutes. The supernatant is filtered by a 0.2-μm filter or other suitable depth filter to remove any small particles that may skipped into the solution. The depth filtrate was proceeded to the next step of UFDF-1. 12-1 shows the SEC-HPLC chromatograms of the neutralized filtrate and the depth filtrate after the flocculation.

### 3. Ultrafiltration/Diafiltration-(UFDF-1)

Purification begins with the depth filtrate (from step 2 above) by ultrafiltration and diafiltration (UFDF) using 10-kDa Sartocon Hydrosart membrane cassette. The amount of material processed is typically 20-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 25 mM Tris/25 mM NaCl pH 7.5. The numbers of diavolumes are 18 for each diafiltration step, respectively. The comparison of SEC-HPLC chromatograms for the depth filtrate and the retentate after the UFDF-1 was analyzed.

### 4. Carbon Filtration

This unit operation reduces the level of host cell impurities such as proteins and nucleic acids as well as colored impurities (see WO2008118752). The 3M R55SP carbon filter is used at loading of approximately 200-250 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the UFDF retentate and the carbon filtrate and carbon bulk, which included rinse indicate that carbon filtration was very effective in removing the residual color and small MW impurities.

### 5. IEX Membrane Chromatography

This step was developed to remove the non-specific negatively charged impurity (see Section IEX membrane Chromatography in Example 5). The IEX membrane used here is Millipure's NatriFlo cassette. Alternatively, the Sartobind Q membrane from Sartorius Stedim can also be used. The membrane was first equilibrated with the 20 mM Tris/20 mM NaCl pH 7.2, typically 20-30 membrane volume (MV). The carbon filtrate was then loaded onto the membrane at about 100-150 mg of O—Ag per mL of MV. The flow through effluent or filtrate that contained the product was collected. The membrane was rinsed with equilibration buffer and two step washings, the first one with 25 mM Tris/25 mM NaCl pH7.4 buffer and the second one with the high salt buffer, 25 mM Tris/1.0M NaCl pH 7.4. The SEC-HPLC chromatograms for the IEX filtrate (the top chromatogram) were analyzed.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent was collected along with the rinse as HIC filtrate, and the water wash was also collected for analysis. The conductivity and UV280 profiles of the HIC chromatographic run were analyzed. A small amount of the hydrophobic substances bound onto the HIC membrane and it was subsequently washed out by the water. The SEC-HPLC chromatogram for the HIC filtrate and the HIC wash was analyzed.

### 7. Ultrafiltration/Diafiltration-(UFDF-2)

This unit operation concentrates the product to the desired concentration and replaces the ammonium sulfate with water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 300 LMH and 0.5-1.0 bars, respectively. The retentate from the UFDF-2 was collected along with the rinse. The final pool was filtered through a 0.2-μm filter. The SEC-HPLC profiles for the final purified O18 O—Ag were analyzed. Table 12-1 below summarizes the quality attributes of the final purified O18 O—Ag.

TABLE 12-1
Summary of Quality Attributes of Purified O18 O-Ag

| | |
|---|---|
| Purity by SEC-HPLC | >99.9% |
| Molecular weight (kDa) | 52.9 |
| Residual Protein (%) | 0.38 |
| Residual Nucleic Acid (%) | 0.04 |
| Endotoxin (EU/mg) | <0.024 |
| NMR structural identification | Conforms |

### Example 17. Purification of E. coli O-Antigen without Flocculation

To further demonstrate the robustness of the purification process for E. coli O-antigen polysaccharides described in the previous 12 examples, here we showed that the same purification process was equally effective for the feed stream without using the Alum solution as a flocculation agent. The serotypes of O2 and O6 and O25b O—Ag were used for demonstration of this clarification process.

### 1. Release of O-Antigen

The process begins with acid hydrolysis after fermentation process to release the O—Ag from the lipopolysaccharides (LPS). Based on this DOE results conducted for the serotype O25b O—Ag (see Example 5), the conditions used for acid hydrolysis for all serotypes of O-antigens are pH 3.8±0.1, temperature 95±5° C. and incubation time of 2.0 hours. This step was performed in the fermentation tank.

### 2. Acid Treatment for Serotype O25b

After the acid hydrolysis, the batch was cooled to the ambient temperature. The pH was further adjusted to 3.2 using sulfuric acid, and the batch was incubated at ambient temperature for 1.0 hour, followed by centrifugation at 12,000-14,000 g for 30 minutes. The supernatant showed slight haziness as opposed to that of flocculated batch, and hazy supernatant was then filtered by a 0.2-μm filter. The resulting depth filtrate was visually clear and proceeded to the subsequent purification processing steps which include UFDF-1, carbon filtration, IEX membrane chromatography, HIC filtration and UFDF-2 described in the Examples 5-16.

Table 13-1 shows the comparison of quality attributes for O25b O—Ag purified with and without Alum flocculation step.

TABLE 13-1
Summary of Quality Attributes of O25b O-Ag
Purified with and without Alum flocculation step.

| | Purified | Purified |
|---|---|---|
| | with | without |
| | Alum | Alum |
| Purity by SEC-HPLC | >99.9% | >99.9 |
| Molecular weight (kDa) | 52.9 | 49.8 |
| Residual Protein (%) | 0.38 | 0.23 |
| Residual Nucleic Acid (%) | 0.04 | 0.08 |
| Endotoxin (EU/mg) | <0.024 | 0.68 |
| NMR structural identification | Conforms | Conforms |

### 3. Acid Treatment for Serotype O2 and O6

The acid treatment for the serotype O2 and O6 O—Ag were performed for the neutralized filtrate, which were obtained by the process described in the Section One of Example 5. The pH of the neutralized filtrate was adjusted to 3.2, and the batch was then incubated at ambient temperature for 1.0 hour followed by centrifugation at 12,000-14,000 g for 30 minutes. The supernatant in both serotypes showed again slight haziness compared to their respective flocculated solution. However, after the supernatant was filtered by a with 0.2-μm filter, the resulting filtrate became visually clear. The depth filtrate was then proceeded to the subsequent purification processing steps which include UFDF-1, carbon filtration, IEX membrane chromatography, HIC filtration and UFDF-2 described in the Examples 5-16.

Tables 13-2 and 13-3 show the comparison of quality attributes for O2 and O6 O—Ag, respectively, purified with and without Alum flocculation step.

TABLE 13-2
Summary of Quality Attributes of Purified O2 O-Ag

| | Purified | Purified |
|---|---|---|
| | with | without |
| | Alum | Alum |
| Purity by SEC-HPLC | >99.9% | >99.9% |
| Molecular weight (kDa) | 46.1 | 46.2 |
| Residual Protein (%) | 0.23 | 0.15 |
| Residual Nucleic Acid (%) | 0.05 | 0.023 |
| Endotoxin (EU/mg) | 3.2 | 2.19 |
| NMR structural identification | Conforms | Conforms |

TABLE 13-3
Summary of Quality Attributes of Purified O6 O-Ag

| | Purified | Purified |
|---|---|---|
| | with | without |
| | Alum | Alum |
| Purity by SEC-HPLC | >99.9% | >99.9 |
| Molecular weight (kDa) | 45.5 | 46.0 |
| Residual Protein (%) | 0.2 | <0.18 |
| Residual Nucleic Acid (%) | 0.03 | 0.036 |
| Endotoxin (EU/mg) | 0.07 | 0.17 |
| NMR structural identification | Conforms | Conforms |

### Example 18: Methods for Purifying N. meningitidis Serogroup A Polysaccharide (Nm-A Poly)

1. Flocculation and Clarification

The process begins with the Neisseria meningitidis cell culture that was heat treated to 55° C. for one hour to release the polysaccharides from the surface of the cell. The cell broth that contains released product is then subject to the flocculation. The main purpose of this step is to precipitate cell debris, host cell proteins and nucleic acids from the broth. It also enhances the efficiency of the downstream clarification unit operation.

The flocculation is achieved by adding the 5M CaCl₂ solution to the fermentation broth to the final CaCl2 concentration of 0.2M. The CaCl2 treated solution was incubated at 50-70° C. for one hour with gentle mixing. After incubation, the batch was cooled to the ambient temperature, and then centrifuged at 15,000×g for 30 minutes at 20° C. The supernatant was filtered by a 0.2-μm filter or another suitable depth filter to remove any small particles that might skipped into the solution. The clarified filtrate was proceeding to the initial purification of UFDF-1.

2. Ultrafiltration/Diafiltration-(UFDF-1)

The clarified filtrate from Step 1 above is further purified through ultrafiltration and diafiltration (UFDF) using 10-kDa Sartocon Hydrosart membrane. The amount of clarified filtrate processed is typically ˜55 g of polysaccharides per m² of membrane area. The purposes of this operation are: (i) volume reduction by concentrating the solution 10-15 folds and (ii) buffer exchange by replacing the fermentation media with desired buffer through diafiltration. The buffers used in this step were 25 mM citrate/50M NaCl pH 6.0 for the first diafiltration, and this was followed by the second diafiltration using 20 mM Tris-HCl pH 8.0. The numbers of diavolumes (DVs) for the two diafiltration steps were 10 and 20, respectively. The retentate from the UFDF was collected and analyzed. The conductivity and UV profiles during the UFDF run indicate that a majority of the small MW as well as UV related small molecular weight impurities were removed during the diafiltration, evidenced by the significant drop on the UV signals of the permeate and the SEC-HPLC chromatograms of the clarified filtrate and the retentate of UFDF-1.

3. Carbon Filtration

This unit operation reduces the level of host cell impurities such as proteins and nucleic acids as well as colored impurities (see WO2008118752). The 3M R32SP carbon filter was used at loading of approximately 300-600 g of Nm-A poly from retentate of UFDF-1 per m² of carbon filter area. The carbon filter was rinsed with the diafiltration buffer of 20 mM Tris-HCl pH 8.0 at approximately 20 liters per m² of filter area. The retentate from UFDF-1 was then filtered via carbon filter at a flow rate of 50-75 LMH (liters per m² per hour) in a single pass mode. The filter was then rinsed with the buffer, and the filtrate including rinse that contained the product was collected as carbon filtrate.

SEC-HPLC chromatograms for the UFDF retentate and the carbon filtrate indicated that the RI and UV280 related impurities were removed, and carbon filtrate became visually colorless.

4. Hydrophobic Interaction Chromatography (HIC)

This unit operation removed any impurities that had hydrophobic characteristics, such as residual lipid polysaccharides (endotoxin). The Sartobind Phenyl membrane was used for the HIC step. The carbon filtrate from Step 3 was treated with 4.0M ammonium sulfate (AS) solution to the final concentration of 1.5M. The phenyl membrane was first equilibrated with the running buffer of 1.5M ammonium sulfate (AS). The AS treated carbon filtrate was pushed through the HIC membrane at flow rate of 0.2-1.0 membrane volume (MV) per min. The HIC membrane was then rinsed with the running buffer, followed by the water wash. The flow through effluent along with the buffer rinse was collected as HIC filtrate, and the water wash was also collected for analysis.

The AKTA Avant chromatography run for the HIC purification was analyzed. The product was in the flow through effluent, and the peak shown in the water wash was non-specified hydrophobic related impurity that bound onto the HIC membrane. SEC-HPLC chromatograms for the carbon filtrate and HIC filtrate indicate that hydrophobic impurity was removed by the HIC filtration step.

5. Ultrafiltration/Diafiltration-(UFDF-2)

This unit operation concentrates the product to the desired concentration and replaces the ammonium sulfate with the desirable buffer or water for conjugation. This step is performed using a 10-kDa molecular weight cutoff (MWCO) Sartocon Hydrosart membrane cassette.

The HIC filtrate was concentrated˜10-15 folds, and then followed by diafiltration using water with ˜10-20 numbers of diavolumes (DV). The cross-flow rate and TMP for the UFDF-2 run were typically set at 100-500 LMH and 0.5-1.5 bars, respectively. The conductivity and the UV280 signals of the permeate as a function of DV during the diafiltration were analyzed. After 10 DVs, the conductivity reached steady state, indicating the completion of buffer exchange.

The comparison of SEC-HPLC chromatograms for HIC filtrate and final purified Nm_A polysaccharide after the UFDF-2 was subject to the 0.2-μm filtration. Table 14 summarizes the quality attributes of the final purified Nm_A polysaccharide.

TABLE 14
Summary of Quality Attributes of Purified
Nm_A polysaccharide

| | |
|---|---|
| Purity by SEC-HPLC | >99.9% |
| Molecular weight (kDa) | 33.8 |
| Residual Protein (%) | 0.0 |
| Residual Nucleic Acid (%) | 0.0 |
| Endotoxin (EU/mg) | 0.87 EU/mg |

### Example 19: Methods for Purifying N. meningitidis Serogroup C Polysaccharide (Nm-C Poly)

1. Flocculation and Clarification

The process begins with the Neisseria meningitidis cell culture that was heat treated to 55° C. for one hour to release the polysaccharides from the surface of the cell. The cell broth that contains released product is then subject to the flocculation. The main purpose of this step is to precipitate cell debris, host cell proteins and nucleic acids from the broth. It also enhances the efficiency of the downstream clarification unit operation.

This is achieved by adding the concentrated CaCl₂ solution to the fermentation brothe and adjust the final CaCl₂ concentration to 0.3M. The CaCl₂ treated solution was incubated at 50° C. for one hour with gentle mixing. After incubation, the batch was cooled to the ambient temperature, and then centrifuged at 12,000-14,000 g for 30 minutes at 20° C. The supernatant was filtered by a 0.2-μm filter or another suitable depth filter to remove any small particles that might skipped into the solution. The clarified filtrate was proceeding to the initial purification of UFDF-1.

2. Ultrafiltration/Diafiltration-(UFDF-1)

The clarified filtrate from Step 1 above is further purified through ultrafiltration and diafiltration (UFDF) using 10-kDa Sartocon Hydrosart membrane. The amount of clarified filtrate processed is typically ˜40 g of polysaccharides per m² of membrane area. The purposes of this operation are: (i) volume reduction by concentrating the solution 10-15 folds and (ii) buffer exchange by replacing the fermentation media with desired buffer through diafiltration. The buffer used in this step is 25 mM citrate/50M NaCl pH 6.0 followed by the second diafiltration of water or other desirable buffers. The numbers of diavolumes were 10-20 for both diafiltration steps. The retentate from the UFDF is collected and analyzed. The conductivity and UV profiles during the UFDF run are reviewed to verify that a majority of the small MW as well as UV related impurities are removed during the first diafiltration, evidenced by the significant drop on the UV signals of the permeate. A comparison of SEC-HPLC chromatograms of the clarified filtrate and the retentate of UFDF-1, for both RI and UV detections is analyzed.

3. Ion Exchange Chromatography (IEX)

The IEX membrane used here is Millipure's NatriFlo membrane cassette. Alternatively, the Sartobind Q membrane from Sartorius Stedim can also be used.

The membrane is first equilibrated with the 20 mM Tris pH 8.0, typically 20-30 membrane volume (MV). The carbon filtrate from previous step is adjusted to 20 mM Tris concentration and is pumped through the membrane at flow rate of 0.2-1.0 MV/min with about 70 mg of polysaccharide per mL of MV. The membrane is then rinsed with 10-30 MV of equilibration buffer. The elution is carried out with linear gradient to 50% of the high salt buffer 20 mM Tris/1.0M NaCl pH 8.0 in about 13 MV and followed by 15 MV to 100% of high salt buffer. The elution fractions that corresponded to the two elution peaks are pooled separately and analyzed by SEC-HPLC. The high molecular weight impurity that showed in the UFDF-1 is captured by the IEX membrane and removed during the first elution. The second elution portion contains mostly the product.

4. Hydrophobic Interaction Chromatography (HIC)

This unit operation removes any impurities that had hydrophobic characteristics, such as residual lipid polysaccharides (endotoxin). The Sartobind Phenyl membrane was used for the HIC step. The eluate from IEX chromatography was treated with 4.0M ammonium sulfate (AS) solution to the final concentration of 1.5M. The phenyl membrane was first equilibrated with the running buffer of 1.5M ammonium sulfate (AS). The AS treated IEX eluate was pushed through the HIC membrane at flow rate of 0.2-1.0 membrane volume (MV) per min. The HIC membrane was then rinsed with the running buffer, followed by the water wash. The flow through effluent along with the buffer rinse was collected as HIC filtrate, and the water wash was also collected for analysis.

The AKTA Avant chromatography run for the HIC purification was analyzed. The product was in the flow through effluent, and the peak shown in the water wash was non-specified hydrophobic related impurity that bound onto the HIC membrane.

5. Ultrafiltration/Diafiltration-(UFDF-2)

This unit operation concentrates the product to the desired concentration and replaces the ammonium sulfate with the desirable buffer or water for conjugation. This step is performed using a 10-kDa molecular weight cutoff (MWCO) Sartocon Hydrosart membrane cassette.

The HIC filtrate was concentrated ˜10-15 folds, and then followed by diafiltration using water with ˜20 numbers of diavolumes (DV). The crossflow rate and TMP for the UFDF-2 run were typically set at 100-500 LMH and 0.5-1.5 bars, respectively. The conductivity and the UV280 signals of the permeate as a function of DV during the diafiltration were analyzed. After 10 DVs, the conductivity reached steady state, indicating the completion of buffer exchange. The final purified Nm_C polysaccharide after UFDF-2 was subject to the 0.2-μm filtration. Table 15 summarizes the quality attributes of the final purified Nm_C polysaccharide.

TABLE 15
Summary of Quality Attributes of Purified
Nm_C polysaccharide

| | |
|---|---|
| Purity by SEC-HPLC | >99.9% |
| Molecular weight (kDa) | 154.9 |
| Residual Protein (%) | 0.0 |
| Residual Nucleic Acid (%) | 0.0 |
| Endotoxin (EU/mg) | 0.18 |

### Example 20: Methods for Purifying N. meningitidis Serogroup W Polysaccharide (Nm-W Poly)

1. Flocculation and Clarification

The process begins with the Neisseria meningitidis cell culture that was heat treated to 55° C. for one hour to release the polysaccharides from the surface of the cell. The cell broth that contains released product is then subject to the flocculation. The main purpose of this step is to precipitate cell debris, host cell proteins and nucleic acids from the broth. It also enhances the efficiency of the downstream clarification unit operation.

This is achieved by adding the 5M CaCl₂ solution to the fermentation broth to adjust the final CaCl2 concentration to 0.2M. The CaCl₂ treated solution was incubated at 50° C. for one hour with gentle mixing. After incubation, the batch was cooled to the ambient temperature, and then centrifuged at 14,000 g for 30 minutes at 20° C. The supernatant was filtered by a 0.2-μm filter or another suitable depth filter to remove any small particles that might skipped into the solution. The clarified filtrate was proceeding to the initial purification of UFDF-1.

2. Ultrafiltration/Diafiltration-(UFDF-1)

The clarified filtrate from Step 1 above is further purified through ultrafiltration and diafiltration (UFDF) using 10-kDa Sartocon Hydrosart membrane. The amount of clarified filtrate processed is about 84 g of polysaccharides per m² of membrane area. The purposes of this operation are: (i) volume reduction by concentrating the solution 10-15 folds and (ii) buffer exchange by replacing the fermentation media with desired buffer through diafiltration. The buffer used in this step was 25 mM citrate/50M NaCl pH 6.0 followed by the second diafiltration with water or other desirable buffers. The numbers of diavolumes were 10 for the first diafiltration and 20 for the second diafiltration, respectively. The retentate from the UFDF was collected and analyzed. The conductivity and UV profiles during the UFDF run indicate that a majority of the small MW as well as UV related impurities were removed during the first diafiltration, evidenced by the significant drop on the UV signals of the permeate.

3. Carbon Filtration

This unit operation reduces the level of host cell impurities such as proteins and nucleic acids as well as colored impurities (see WO2008118752). The 3M R32SP carbon filter was used at loading of approximately 1,000 g of Nm-W poly from retentate of UFDF-1 per m² of carbon filter area. The carbon filter was first rinsed with water followed by the diafiltration buffer at approximately 20 liters of buffer per m² of filter area. The retentate from UFDF-1 was then filtered at a flow rate of 60 LMH (liters per m² per hour) in a single pass mode. The filter was then rinsed with the buffer, and the filtrate including rinse that contained the product was collected as carbon filtrate.

SEC-HPLC chromatograms for the UFDF retentate and the carbon filtrate indicated that the RI, UV280 related and small molecular weight impurities were removed by the carbon filter. The carbon filtrate became visually colorless.

4. Hydrophobic Interaction Chromatography (HIC)

This unit operation removes any impurities that had hydrophobic characteristics, such as residual lipid polysaccharides (endotoxin). The Sartobind Phenyl membrane was used for the HIC step. The carbon filtrate from Step 3 was treated with 4.0M ammonium sulfate (AS) solution to the final concentration of 1.5-2.0M. The amount of Nm_W polysaccharides loaded onto the HIC membrane was about 30-116 mg per mL of membrane volume (MV). The phenyl membrane was first equilibrated with the running buffer of ammonium sulfate (AS). The AS treated carbon filtrate was pushed through the HIC membrane at flow rate of 0.2-1.0 membrane volume (MV) per min. The HIC membrane was then rinsed with the running buffer, followed by the water wash. The flow through effluent along with the buffer rinse was collected as HIC filtrate, and the water wash was also collected for analysis.

The AKTA Avant chromatography run for the HIC purification was analyzed. The product was in the flow through effluent, and the peak shown in the water wash was non-specified hydrophobic related impurity that bound onto the HIC membrane. SEC-HPLC chromatograms for the carbon filtrate and HIC filtrate indicate that hydrophobic impurity was removed by the HIC filtration step.

5. Ultrafiltration/Diafiltration-(UFDF-2)

This unit operation concentrates the product to the desired concentration and replaces the ammonium sulfate with the desirable buffer or water for conjugation. This step is performed using a 10-kDa molecular weight cutoff (MWCO) Sartocon Hydrosart membrane cassette.

The HIC filtrate was concentrated ˜10-15 folds, and then followed by diafiltration using water with ˜10-20 numbers of diavolumes (DV). The crossflow rate and TMP for the UFDF-2 run were typically set at 100-500 LMH and 0.5-1.5 bars, respectively. The conductivity and the UV280 signals of the permeate as a function of DV during the diafiltration were analyzed. After 10 DVs, the conductivity reached steady state, indicating the completion of buffer exchange.

The comparison of SEC-HPLC chromatograms for HIC filtrate and final purified Nm_W polysaccharide after the UFDF-2 was subject to the 0.2-μm filtration. Table 16 summarizes the quality attributes of the final purified Nm_W polysaccharide.

TABLE 16
Summary of Quality Attributes of Purified
Nm_W polysaccharide

| | |
|---|---|
| Purity by SEC-HPLC | >99.9% |
| Molecular weight (kDa) | 216.8 |
| Residual Protein (%) | 0.0 |
| Residual Nucleic Acid (%) | 0.0 |
| Endotoxin (EU/mg) | 0.07 EU/mg |

### Example 21: Methods for Purifying N. meningitidis Serogroup Y Polysaccharide (Nm-Y Poly)

1. Flocculation and Clarification

The process begins with the Neisseria meningitidis cell culture that was heat treated to 55° C. for one hour to release the polysaccharides from the surface of the cell. The cell broth that contains released product is then subject to the flocculation. The main purpose of this step is to precipitate cell debris, host cell proteins and nucleic acids from the broth. It also enhances the efficiency of the downstream clarification unit operation.

This is achieved by adding the 5M CaCl₂ solution to the fermentation broth to adjust the final CaCl₂ concentration to 0.2M. The CaCl₂ treated solution was incubated at 50-70° C. for one hour with gentle mixing. After incubation, the batch was cooled to the ambient temperature, and then centrifuged at 15,000 g for 40 minutes at 20° C. The supernatant was filtered by a 0.2-μm filter or another suitable depth filter to remove any small particles that might skipped into the solution. The clarified filtrate was proceeding to the initial purification of UFDF-1.

2. Ultrafiltration/Diafiltration-(UFDF-1)

The clarified filtrate from Step 1 above is further purified through ultrafiltration and diafiltration (UFDF) using 10-kDa Sartocon Hydrosart membrane. The amount of clarified filtrate processed is about 20 g of polysaccharides per m² of membrane area. The purposes of this operation are: (i) volume reduction by concentrating the solution 10-15 folds and (ii) buffer exchange by replacing the fermentation media with desired buffer through diafiltration. The buffer used in this step was 25 mM citrate/50M NaCl pH 6.0 followed by the second diafiltration with 20 mM Tris-HCl/0.1M NaCl pH 8.0. The numbers of diavolumes were 12 for the first diafiltration and 25 for the second diafiltration, respectively. The retentate from the UFDF was collected and analyzed. The conductivity and UV profiles during the UFDF run indicate that a majority of the small MW as well as UV related impurities were removed during the first diafiltration, evidenced by the significant drop on the UV signals of the permeate.

3. Carbon Filtration

This unit operation reduces the level of host cell impurities such as proteins and nucleic acids as well as colored impurities (see WO2008118752). The 3M R32SP carbon filter was used at loading of approximately 885 g of Nm-Y poly from retentate of UFDF-1 per m² of carbon filter area. The carbon filter was first rinsed with water followed by the diafiltration buffer at approximately 20 liters of buffer per m² of filter area. The retentate from UFDF-1 was then filtered at a flow rate of 72 LMH (liters per m² per hour) in a single pass mode. The filter was then rinsed with the buffer, and the filtrate including rinse that contained the product was collected as carbon filtrate.

SEC-HPLC chromatograms for the UFDF retentate and the carbon filtrate indicated that the RI, UV280 related and small molecular weight impurities were removed by the carbon filter. The carbon filtrate became visually colorless.

4. Hydrophobic Interaction Chromatography (HIC)

This unit operation removes any impurities that had hydrophobic characteristics, such as residual lipid polysaccharides (endotoxin). The Sartobind Phenyl membrane was used for the HIC step. The carbon filtrate from Step 3 was treated with 4.0M ammonium sulfate (AS) solution to the final concentration of 1.75M. The amount of Nm_Y polysaccharides loaded onto the HIC membrane was about 40 mg per mL of membrane volume (MV). The phenyl membrane was first equilibrated with the running buffer of ammonium sulfate (AS). The AS treated carbon filtrate was pushed through the HIC membrane at flow rate of 0.2-1.0 membrane volume (MV) per min. The HIC membrane was then rinsed with the running buffer, followed by the water wash. The flow through effluent along with the buffer rinse was collected as HIC filtrate, and the water wash was also collected for analysis.

The AKTA Avant chromatography run for the HIC purification was analyzed. The product was in the flow through effluent, and the peak shown in the water wash was non-specified hydrophobic related impurity that bound onto the HIC membrane. SEC-HPLC chromatograms for the carbon filtrate and HIC filtrate indicate that hydrophobic related impurity was removed by the HIC filtration step.

5. Ultrafiltration/Diafiltration-(UFDF-2)

This unit operation concentrates the product to the desired concentration and replaces the ammonium sulfate with the desirable buffer or water for conjugation. This step is performed using a 10-kDa molecular weight cutoff (MWCO) Sartocon Hydrosart membrane cassette.

The HIC filtrate was concentrated ˜10-15 folds, and then followed by diafiltration using water with ˜25 numbers of diavolumes (DV). The crossflow rate and TMP for the UFDF-2 run were typically set at 300-400 LMH and 0.5-1.5 bars, respectively. The conductivity and the UV280 signals of the permeate as a function of DV during the diafiltration were analyzed. After 10 DVs, the conductivity reached steady state, indicating the completion of buffer exchange.

The comparison of SEC-HPLC chromatograms for HIC filtrate and final purified Nm_Y polysaccharide after the UFDF-2 was subject to the 0.2-μm filtration. Table 17 summarizes the quality attributes of the final purified Nm_Y polysaccharide.

TABLE 17
Summary of Quality Attributes of Purified
Nm_Y polysaccharide

| | |
|---|---|
| Purity by SEC-HPLC | >99.9% |
| Molecular weight (kDa) | 247.0 |
| Residual Protein (%) | 0.0 |
| Residual Nucleic Acid (%) | 0.0 |
| Endotoxin (EU/mg) | 0.35 EU/mg |

### Example 22: Descriptions of Purification for Klebsiella O-Antigen Polysaccharides

### 1. Release of O-Antigen

The Klebsiella O1 and O2 O-antigens (Kleb O—Ag) are short chain O-antigen and the molecular weight is expected to be in the range of 8.0-16.0 kDa. The purification process described in Examples 5-17 for E. coli O—Ag also applies to the Kleb O—Ag. After fermentation, the Klebsiella O1 and O2 O-antigen is released from the lipopolysaccharide (LPS) through the acid hydrolysis at the pH 3.8±0.1, temperature and 95° C.±5° C. and incubation time of 2.0 hours. This step was performed in the fermentation tank. This condition will cleave the acid-labile bond between lipid-A and the core oligosaccharide of the LPS (see Example 5).

### 2. Flocculation

Following the release of Kleb O—Ag as described in step 1 above, the broth is cooled to the ambient temperature and treated with 10% Alum solution to the final concentration of 2.0% (w/v) and the pH was further adjusted to 3.2. This flocculation step will precipitate cell debris, host cell proteins and nucleic acids. The flocculated slurry was incubated at ambient temperature for 1.0 hour, followed by centrifugation at 12,000-14,000 g for 30 minutes. The supernatant was filtered by a 0.2-μm filter or other suitable depth filter to removed any small particles that may skipped in to the solution. The depth filtrate was proceeded to the next step of UFDF-1.

### 3. Ultrafiltration/Diafiltration-(UFDF-1)

Purification begins with the depth filtrate (from step 2 above) by ultrafiltration and diafiltration (UFDF) using 5-kDa or 10 kDa Sartocon Hydrosart membrane cassette. The amount of material processed was typically 15-30 liters per m² of membrane area. The purposes of this operation are: (i) volume reduction by concentrating the solution 10-20 folds and (ii) buffer exchange by replacing the fermentation media with desired buffer through diafiltration. The buffer used in this step is 20 mM citrate/0.1 M NaCl pH 6.0 followed by the second buffer of 20 mM Tris/20 mM NaCl pH 7.2. The numbers of diavolumes are 10-18 for each diafiltration step, respectively. The SEC-HPLC chromatograms of retentate after the UFDF-1 were analyzed.

### 4. Carbon Filtration

This unit operation reduces the level of host cell impurities such as proteins and nucleic acids as well as colored impurities (see WO2008118752). The 3M carbon filter is used at loading of approximately 100-150 g of O—Ag per m² of carbon filter area. The carbon was first rinsed with water followed by the diafiltration buffer at approximately 20 liters of buffer per m² of membrane area. The retentate from UFDF-1 was then filtered at a flow rate of 50 LMH (liters per m² per hour) in single pass mode. The carbon filter was then rinsed with buffer. The filtrate and the buffer rinse that contained the product was collected.

The SEC-HPLC chromatograms for the carbon filtrate were analyzed. The fact that the product peak was reduced after the carbon filtration, indicating the non-specific adsorption mode by which carbon filter was designed for. Nevertheless, the color related impurities were mostly removed.

### 5. IEX Membrane Chromatography

This step was developed to remove the non-specific negatively charged impurity (see Section IEX membrane Chromatography in Example 5). The IEX membrane used here is Millipure's NatriFlo (HD-Q) cassette. Alternatively, the Sartobind Q membrane from Sartorius Stedim or Emphaze AEX Hybrid Purifier from 3M can also be used. The membrane was first equilibrated with the 20 mM Tris/20 mM NaCl pH 7.2, typically 20-30 membrane volume (MV). The carbon filtrate was then loaded onto the membrane at about 75-250 mg of O—Ag per mL of MV. The flow through effluent or filtrate that contained the product was collected. The membrane was rinsed with equilibration buffer and then washed with the high salt buffer, 20 mM Tris/1.0M NaCl pH 7.2.

The conductivity and UV profiles of the IEX membrane chromatographic run was analyzed. In this profile, the UV signal showed a peak during the high salt wash, indicating there was an unknown negatively charged impurity that was present in the carbon filtrate. The SEC-HPLC chromatograms for the carbon filtrate, IEX filtrate and the high salt wash effluent indicate that the high salt elution sample high salt wash sample contained large molecular weight impurity.

### 6. Hydrophobic Interaction Chromatography (HIC)

This unit operation removes any impurities that had hydrophobic characteristics, such as residual lipid A left from the acid hydrolysis step. The Sartobind Phenyl 150-mL membrane was used for the HIC step. The IEX filtrate was treated with 4.0M ammonium sulfate (AS) solution to the final concentration of 2.0M. The phenyl membrane was first equilibrated with the running buffer of 2.0M ammonium sulfate. The AS treated IEX filtrate was pushed through the HIC membrane at flow rate of 40-60 mL/min. The HIC membrane was then rinse with the running buffer, followed by the water wash. The flow through effluent along with the buffer rinse was collected as HIC filtrate, and the water wash was also collected for analysis. The chromatographic profiles of UV and conductivity for the HIC membrane filtration.

SEC-HPLC chromatograms for the IEX filtrate, HIC filtrate, HIC water wash and purified O8 O—Ag indicate that the water wash sample showed no visible peak in the RI detection, indicating that there was no hydrophobic related substance present in the IEX stream.

### 7. Ultrafiltration/Diafilatration (UFDF-2)

This unit operation concentrates the product to the desired concentration and replaces the ammonium sulfate with the desirable buffer or water for conjugation. This step is performed using a 5-kDa molecular weight cutoff filter.

The HIC filtrate was concentrated ˜10-folds, and then followed by diafiltration using water with ˜20 numbers of diavolumes (DV). The cross-flow rate and TMP for the UFDF-2 run were typically set at 200 LMH and 0.5 bars, respectively. The retentate from the UFDF-2 was collected along with the rinse. The final pool was filtered through a 0.2-μm filter.

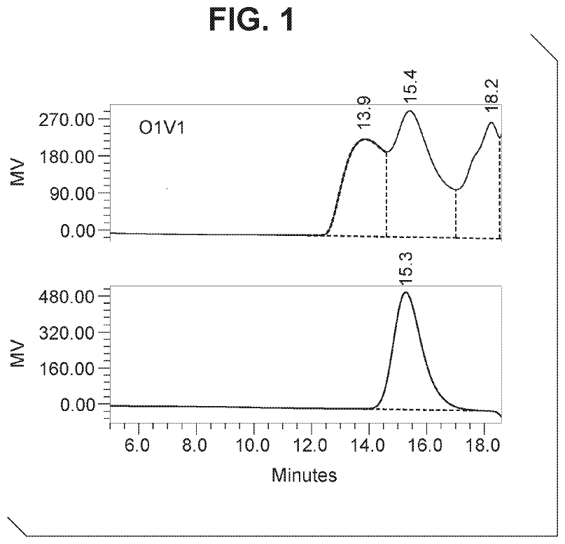

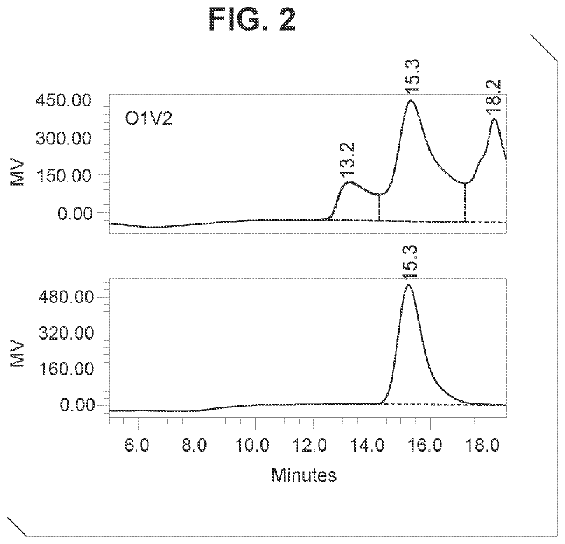

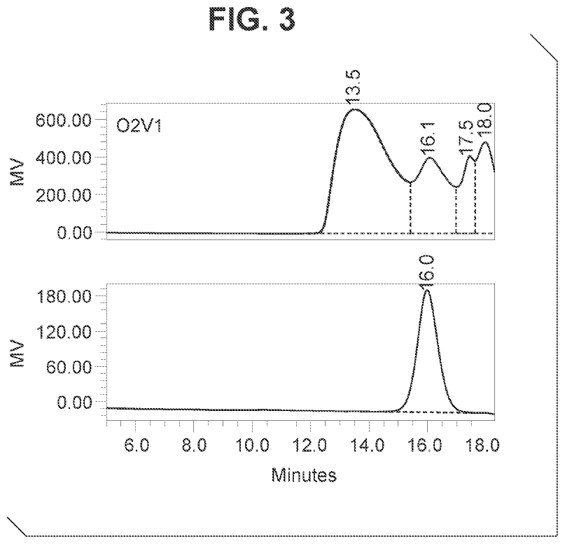

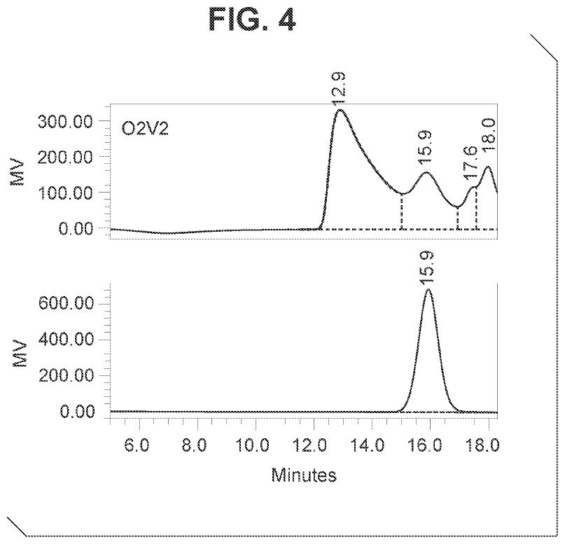

The SEC-HPLC chromatograms of post released O—Ag in broth and of the final purified Kₚ O—Ag of four variants after the purification show that the platform based purification process developed for the E. coli O—Ag is effective to produce high quality product (FIGS. 1-4).

Table 18 provides a summary of quality attributes of the purified Kₚ O—Ag produced by the native Kₚ strain.

TABLE 18
Summary of Quality Attributes of Purified
O1 and O2 Klebsiella O-Ags

| | O1V1 | O1V2 | O2V1 | O2V2 |
|---|---|---|---|---|
| Purity by SEC-HPLC | >99.9 | >99.9 | >99.9 | >99.9 |
| Molecular weight (kDa) | 8.6 | 16.1 | 8.0 | 12.1 |
| Residual Protein (%) | 0.43 | 0.13 | 1.19 | 0.76 |
| Residual Nucleic Acid (%) | 0.14 | 0.05 | 0.14 | 0.06 |
| Endotoxin (EU/mg) | 4.3 | 1.1 | 0.38 | 0.15 |
| NMR structural | Conforms | Conforms | Conforms | Conforms |
| identification | | | | |

Further embodiments of the invention are set out in the following numbered clauses:
clause 1. A method for purifying a bacterial polysaccharide from a solution comprising said polysaccharide together with contaminants, wherein said method comprises a flocculation step.
clause 2. The method of clause 1 wherein the flocculating agent comprises a multivalent cation.
clause 3. The method of clause 2 wherein said multivalent cation is selected from the group consisting of aluminium, iron, calcium and magnesium.
clause 4. The method of clause 2 wherein said flocculating agent is a mixture of at least two multivalent cations selected from the group consisting of aluminium, iron, calcium and magnesium.
clause 5. The method of clause 2 wherein said flocculating agent is a mixture of at least three multivalent cations selected from the group consisting of aluminium, iron, calcium and magnesium.
clause 6. The method of clause 2 wherein said flocculating agent is a mixture of four multivalent cations consisting of aluminium, iron, calcium and magnesium.
clause 7. The method of clause 1 wherein the flocculating agent comprises an agent selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate, calcium chloride, and sodium silicate.
clause 8. The method of clause 1 wherein the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
clause 9. The method of clause 1 wherein the flocculating agent is polyethylenimine (PEI).
clause 10. The method of clause 1 wherein the flocculating agent comprises alum.
clause 11. The method of clause 1 wherein the flocculating agent is alum.
clause 12. The method of clause 1 wherein the flocculating agent comprises potassium alum.
clause 13. The method of clause 1 wherein the flocculating agent potassium alum.
clause 14. The method of clause 1 wherein the flocculating agent comprises sodium alum.
clause 15. The method of clause 1 wherein the flocculating agent is sodium alum.
clause 16. The method of clause 1 wherein the flocculating agent comprises ammonium alum.
clause 17. The method of clause 1 wherein the flocculating agent is ammonium alum.
clause 18. The method of clause 1 wherein the flocculating agent is a mixture of two agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
clause 19. The method of clause 1 wherein the flocculating agent is a mixture of three agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate.
clause 20. The method of clause 1 wherein the flocculating agent is a mixture of four agents selected from the group consisting of alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.
clause 21. The method of clause 1 wherein the flocculating agent comprises an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).
clause 22. The method of clause 1 wherein the flocculating agent is an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts).
clause 23. The method of any one of clauses 1-22 wherein the concentration of flocculating agent is between about 0.1 and about 20% (w/v).
clause 24. The method of any one of clauses 1-22 wherein the concentration of flocculating agent is between about 0.5 and about 10% (w/v).
clause 25. The method of any one of clauses 1-22 wherein the concentration of flocculating agent is between about 1 and about 5% (w/v).
clause 26. The method of any one of clauses 1-22 wherein the concentration of flocculating agent is about 0.1, about 0.25, about 0.5, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10% (w/v).
clause 27. The method of any one of clauses 1-22 wherein the concentration of flocculating agent is about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5 or about 20.0% (w/v)
clause 28. The method of any one of clauses 1-22 wherein the concentration of flocculating agent is about 0.5, about 1.0, about 1.5, about 2.0, about 2.5, about 3.0, about 3.5, about 4.0, about 4.5 or about 5.0% (w/v)
clause 29. The method of any one of clauses 1-22 wherein the concentration of flocculating agent is about 1.0, about 1.5, about 2.0, about 2.5, about 3.0, about 3.5 or about 4.0% (w/v) is used.
clause 30. The method of any one of clauses 1-29 wherein the flocculating agent is added over a period of between a few seconds (e.g. 1 to 10 seconds) and about one month.
clause 31. The method of any one of clauses 1-29 wherein the flocculating agent is added over a period between about 2 seconds and about two weeks.
clause 32. The method of any one of clauses 1-29 wherein the flocculating agent is added over a period of between about 1 minute and about one week.
clause 33. The method of any one of clauses 1-29 wherein the flocculating agent is added over a period of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.
clause 34. The method of any one of clauses 1-29 wherein the flocculating agent is added over a period of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
clause 35. The method of any one of clauses 1-29 wherein the flocculating agent is added over a period of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
clause 36. The method of any one of clauses 1-29 wherein the flocculating agent is added over a period of between about 15 minutes and about 3 hours.
clause 37. The method of any one of clauses 1-29 wherein the flocculating agent is added over a period of between about 30 minutes and about 120 minutes.
clause 38. The method of any one of clauses 1-29 wherein the flocculating agent is added over a period of about 2 seconds, about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3.0 hours, about 3.5 hours, about 4.0 hours, about 4.5 hours, about 5.0 hours, about 5.5 hours, about 6.0 hours, about 6.5 hours, about 7.0 hours, about 7.5 hours, about 8.0 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.
clause 39. The method of any one of clauses 1-38 wherein the flocculating agent is added without agitation.
clause 40. The method of any one of clauses 1-38 wherein the flocculating agent is added under agitation.
clause 41. The method of any one of clauses 1-38 wherein the flocculating agent is added under gentle agitation.
clause 42. The method of any one of clauses 1-38 wherein the flocculating agent is added under vigorous agitation.
clause 43. The method of any one of clauses 1-42 wherein the solution is hold for some time to allow settling of the flocs prior to downstream processing.
clause 44. The method of any one of clauses 1-43 wherein the flocculation step is performed with a settling time of between a few seconds (e.g. 2 to 10 seconds) to about 1 minute.
clause 45. The method of any one of clauses 1-43 wherein the flocculation step is performed with a settling time of at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155 or at least about 160 minutes.
clause 46. The method of clause 1-43 wherein the settling time is less than a week.
clause 47. The method of any one of clauses 1-43 wherein the flocculation step is performed with a settling time of between about 1, about 2, about 3, about 4, about 5, about 6, about 7, about 8, about 9, about 10, about 15, about 20, about 25, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380, about 1440 minute(s), about two days, about three days, about four days, about five days or about six days and 1 week.
clause 48. The method of any one of clauses 1-43 wherein the flocculation step is performed with a settling time of between a few seconds (e.g. 1 to 10 seconds) and about one month.
clause 49. The method of any one of clauses 1-43 wherein the flocculation step is performed with a settling time of between about 2 seconds and about two weeks.
clause 50. The method of any one of clauses 1-43 wherein the flocculation step is performed with a settling time of between about 1 minute and about one week.
clause 51. The method of any one of clauses 1-43 wherein the flocculation step is performed with a settling time of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.
clause 52. The method of any one of clauses 1-43 wherein the flocculation step is performed with a settling time of between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
clause 53. The method of any one of clauses 1-43 wherein the flocculation step is performed with a settling time of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.
clause 54. The method of any one of clauses 1-43 wherein the flocculation step is performed with a settling time of between about 15 minutes and about 3 hours.
clause 55. The method of any one of clauses 1-43 wherein the flocculation step is performed with a settling time of between about 30 minutes and about 120 minutes.
clause 56. The method of any one of clauses 1-43 wherein the flocculation step is performed with a settling time of about 10 seconds, about 30 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.
clause 57. The method of any one of clauses 1-43 wherein the flocculation step is performed with a settling time of about 5, about 10, about 15, about 20, about 25, about 30, about 60, about 90, about 120, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 or about 1440 minute(s) and two days.
clause 58. The method of any one of clauses 1-43 wherein the flocculation step is performed with a settling time of between about 5 minutes and about one day.
clause 59. The method of any one of clauses 1-43 wherein the flocculation step is performed with a settling time of between about 5 minutes and about 120 minutes.
clause 60. The method of any one of clauses 1-43 wherein the flocculation step is performed with a settling time of about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.
clause 61. The method of any one of clauses 43-60 wherein the settling step is conducted without agitation.
clause 62. The method of any one of clauses 43-60 wherein the settling step is conducted under agitation.
clause 63. The method of any one of clauses 43-60 wherein the settling step is conducted under gentle agitation.
clause 64. The method of any one of clauses 43-60 wherein the settling step is conducted under vigorous agitation.
clause 65. The method of any one of clauses 1-64 wherein said flocculation step is performed at an acidic pH.
clause 66. The method of any one of clauses 1-64 wherein said flocculation step is performed at a pH below 7.0, 6.0, 5.0 or 4.0.
clause 67. The method of any one of clauses 1-64 wherein said flocculation step is performed at a pH between 7.0 and 1.0.
clause 68. The method of any one of clauses 1-64 wherein said flocculation step is performed at a pH between 5.5 and 2.5, 5.0 and 2.5, 4.5 and 2.5, 4.0 and 2.5, 5.5 and 3.0, 5.0 and 3.0, 4.5 and 3.0, 4.0 and 3.0, 5.5 and 3.5, 5.0 and 3.5, 4.5 and 3.5 or 4.0 and 3.5.
clause 69. The method of any one of clauses 1-64 wherein said flocculation step is performed at a pH of about 5.5, about 5.0, about 4.5, about 4.0, about 3.5, about 3.0, about 2.5, about 2.0, about 1.5 or about 1.0.
clause 70. The method of any one of clauses 1-64 wherein said flocculation step is performed at a pH of about 4.0, about 3.5, about 3.0 or about 2.5.
clause 71. The method of any one of clauses 1-64 wherein said flocculation step is performed at a pH of about 3.5.
clause 72. The method of any one of clauses 65-71 wherein said acidic pH is obtained by acidifying the solution with an acid.
clause 73. The method of any one of clauses 65-71 wherein said acidic pH is obtained by acidifying the solution with an acid selected from the group consisting of HCl, H₃PO₄, citric acid, acetic acid, nitrous acid, and sulfuric acid.
clause 74. The method of any one of clauses 65-71 wherein said acidic pH is obtained by acidifying the solution with an amino acid.
clause 75. The method of any one of clauses 65-71 wherein said acidic pH is obtained by acidifying the solution with an amino acid selected from the group consisting of glycine, alanine and glutamate.
clause 76. The method of any one of clauses 65-71 wherein said acidic pH is obtained by acidifying the solution with sulfuric acid.
clause 77. The method of any one of clauses 65-71 wherein the acid is added under agitation.
clause 78. The method of any one of clauses 65-71 wherein the acid is added under gentle agitation.
clause 79. The method of any one of clauses 65-71 wherein the acid is added under vigorous agitation.
clause 80. The method of any one of clauses 1-79 wherein the addition of the flocculating agent is performed at a temperature between about 4° C. and about 30° C.
clause 81. The method of any one of clauses 1-79 wherein the addition of the flocculating agent is performed at a temperature of about 4C, about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 82. The method of any one of clauses 1-79 wherein the addition of the flocculating agent is performed at a temperature of about 20° C.
clause 83. The method of any one of clauses 1-79 wherein the addition of the flocculating agent is performed at a temperature of between about 30° C. to about 95° C.
clause 84. The method of any one of clauses 1-79 wherein the addition of the flocculating agent is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 85. The method of any one of clauses 1-79 wherein the addition of the flocculating agent is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 86. The method of any one of clauses 1-79 wherein the addition of the flocculating agent is performed at a temperature of about 50° C.
clause 87. The method of any one of clauses 43-86 wherein the settling step, if present, is performed at a temperature between about 4° C. and about 30° C.
clause 88. The method of any one of clauses 43-86 wherein the settling step, if present, is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 89. The method of any one of clauses 43-86 wherein the settling step, if present, is performed at a temperature of about 20° C.
clause 90. The method of any one of clauses 43-86 wherein the settling step, if present, is performed at a temperature of between about 30° C. to about 95° C.
clause 91. The method of any one of clauses 43-86 wherein the settling step, if present, is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 92. The method of any one of clauses 43-86 wherein the settling step, if present, is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 93. The method of any one of clauses 43-86 wherein the settling step, if present, is performed at a temperature of about 50° C.
clause 94. The method of any one of clauses 72-93 wherein the acidification step, if present, is performed at a temperature between about 4° C. and about 30° C.
clause 95. The method of any one of clauses 72-93 wherein the acidification step, if present, is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 96. The method of any one of clauses 72-93 wherein the acidification step, if present, is performed at a temperature of about 20° C.
clause 97. The method of any one of clauses 72-93 wherein the acidification step, if present, is performed at a temperature of between about 30° C. to about 95° C.
clause 98. The method of any one of clauses 72-93 wherein the acidification step, if present, is performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 99. The method of any one of clauses 72-93 wherein the acidification step, if present, is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 100. The method of any one of clauses 72-93 wherein the acidification step, if present, is performed at a temperature of about 50° C.
clause 101. The method of any one of clauses 1-79 wherein the addition of the flocculating agent and the settling step, if present, are performed at a temperature between about 40C and about 30° C.
clause 102. The method of any one of clauses 1-79 wherein the addition of the flocculating agent and the settling step, if present, are performed at a temperature of about 4C, about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 103. The method of any one of clauses 1-79 wherein the addition of the flocculating agent and the settling step, if present, are performed at a temperature of about 20° C.
clause 104. The method of any one of clauses 1-79 wherein the addition of the flocculating agent and the settling step, if present, are performed at a temperature of between about 30° C. to about 95° C.
clause 105. The method of any one of clauses 1-79 wherein the addition of the flocculating agent and the settling step, if present, are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 106. The method of any one of clauses 1-79 wherein the addition of the flocculating agent and the settling step, if present, are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 107. The method of any one of clauses 1-79 wherein the addition of the flocculating agent and the settling step, if present, are performed at a temperature of about 50° C.
clause 108. The method of any one of 72-79 wherein the addition of the flocculating agent and the acidification step are performed at a temperature between about 40C and about 30° C.
clause 109. The method of any one of clauses 72-79 wherein the addition of the flocculating agent and the acidification step are performed at a temperature of about 4C, about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 110. The method of any one of clauses 72-79 wherein the addition of the flocculating agent and the acidification step are performed at a temperature of about 20° C.
clause 111. The method of any one of clauses 72-79 wherein the addition of the flocculating agent and the acidification step are performed at a temperature of between about 30° C. to about 95° C.
clause 112. The method of any one of clauses 72-79 wherein the addition of the flocculating agent and the acidification step are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 113. The method of any one of clauses 72-79 wherein the addition of the flocculating agent and the acidification step are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 114. The method of any one of clauses 72-79 wherein the addition of the flocculating agent and the acidification step are performed at a temperature of about 50° C.
clause 115. The method of any one of clauses 72-79 wherein the addition of the flocculating agent, the settling and acidification steps are performed at a temperature between about 40C and about 30° C.
clause 116. The method of any one of clauses 72-79 wherein the addition of the flocculating agent, the settling and acidification steps are performed at a temperature of about 4C, about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C.
clause 117. The method of any one of clauses 72-79 wherein the addition of the flocculating agent, the settling and acidification steps are performed at a temperature of about 20° C.
clause 118. The method of any one of clauses 72-79 wherein the addition of the flocculating agent, the settling and acidification steps are performed at a temperature of between about 30° C. to about 95° C.
clause 119. The method of any one of clauses 72-79 wherein the addition of the flocculating agent, the settling and acidification steps are performed at a temperature of between about 35° C. to about 80° C., at temperature of between about 40° C. to about 70° C., at temperature of between about 45° C. to about 65° C., at temperature of between about 50° C. to about 60° C., at temperature of between about 50° C. to about 55° C., at temperature of between about 45° C. to about 55° C. or at temperature of between about 45° C. to about 55° C.
clause 120. The method of any one of clauses 72-79 wherein the addition of the flocculating agent, the settling and acidification steps are performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 121. The method of any one of clauses 72-79 wherein the addition of the flocculating agent, the settling and acidification steps are performed at a temperature of about 50° C.
clause 122. The method of any one of clauses 1-71, 80-93 or 101-107 wherein the flocculation step comprises adding a flocculating agent without pH adjustment.
clause 123. The method of any one of clauses 1-122 wherein the flocculation step comprises adding a flocculating agent, adjusting the pH and settling the solution.
clause 124. The method of clause 123 wherein, the flocculating agent is added before adjusting the pH.
clause 125. The method of clause 123 wherein, the pH is adjusted before adding the flocculating agent.
clause 126. The method of clause 123 wherein, the pH is adjusted before adding the flocculating agent and settling the solution.
clause 127. The method of clause 123 wherein, the flocculating agent is added and the solution is settled before adjusting the pH.
clause 128. The method of any one of clauses 1-127 wherein, following flocculation the suspension is clarified by decantation, sedimentation, filtration or centrifugation.
clause 129. The method of any one of clauses 1-127 wherein, following flocculation the suspension is clarified by decantation.
clause 130. The method of any one of clauses 1-127 wherein, following flocculation the suspension is clarified by hydrocyclone.
clause 131. The method of any one of clauses 1-127 wherein, following flocculation the suspension is clarified by sedimentation.
clause 132. The method of any one of clauses 1-127 wherein, following flocculation the suspension is clarified by flotation.
clause 133. The method of any one of clauses 1-127 wherein, following flocculation the suspension is clarified by filtration
clause 134. The method of any one of clauses 1-127 wherein, following flocculation the suspension is clarified by centrifugation.
clause 135. The method of any one of clauses 127-134 wherein, the polysaccharide-containing solution is collected for storage.
clause 136. The method of any one of clauses 127-134 wherein, the polysaccharide-containing solution is collected for additional processing.
clause 137. The method of any one of clauses 127-134 wherein, the polysaccharide-containing solution is stored and then additionally processed.
clause 138. The method of any one of clauses 134-137 wherein, said centrifugation is continuous centrifugation.
clause 139. The method of any one of clauses 134-137 wherein, said centrifugation is bucket centrifugation.
clause 140. The method of any one of clauses 134-139 wherein, the suspension is centrifuged at about 1,000 g, about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g.
clause 141. The method of any one of clauses 134-139 wherein, the suspension is centrifuged at about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g or about 25,000 g.
clause 142. The method of any one of clauses 134-139 wherein, the suspension is centrifuged between about 5,000 g and about 25,000 g.
clause 143. The method of any one of clauses 134-139 wherein, the suspension is centrifuged between about 8,000 g and about 20,000 g.
clause 144. The method of any one of clauses 134-139 wherein, the suspension is centrifuged between about 10,000 g and about 15,000 g.
clause 145. The method of any one of clauses 134-139 wherein, the suspension is centrifuged between about 10,000 g and about 12,000 g.
clause 146. The method of any one of clauses 134-145 wherein, the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes.
clause 147. The method of any one of clause 146 wherein, the suspension is centrifuged during less than 24 hours.
clause 148. The method of any one of clauses 134-145 wherein, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320 or about 1380 minutes and 1440 minutes.
clause 149. Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes.
clause 150. The method of any one of clauses 134-145 wherein, the suspension is centrifuged during between about 5 minutes and about 3 hours.
clause 151. The method of any one of clauses 134-145 wherein, the suspension is centrifuged during between about 5 minutes and about 120 minutes.
clause 152. The method of any one of clauses 134-145 wherein, the suspension is centrifuged during between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes or about 155 minutes and about 160 minutes.
clause 153. The method of any one of clauses 134-145 wherein, the suspension is centrifuged during between about 10 minutes, about 15 minutes, about 20 minutes, about 25 minutes, about 30 minutes, about 35 minutes, about 40 minutes, about 45 minutes, about 50 minutes or about 55 minutes and about 60 minutes.
clause 154. The method of any one of clauses 134-145 wherein, the suspension is centrifuged during about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 minutes or about 1440 minutes.
clause 155. The method of any one of clauses 134-145 wherein, the suspension is centrifuged during about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.
clause 156. The method of any one of clauses 134-138 or 140-155 wherein, said centrifugation is continuous centrifugation and the feed rate is between 50-5000 ml/min, 100-4000 ml/min, 150-3000 ml/min, 200-2500 ml/min, 250-2000 ml/min, 300-1500 ml/min, 300-1000 ml/min, 200-1000 ml/min, 200-1500 ml/min, 400-1500 ml/min, 500-1500 ml/min, 500-1000 ml/min, 500-2000 ml/min, 500-2500 ml/min or 1000-2500 ml/min.
clause 157. The method of any one of clauses 134-138 or 140-155 wherein, said centrifugation is continuous centrifugation and the feed rate is about 10, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1650 about 1700, about 1800, about 1900, about 2000, about 2100, about 2200, about 2300, about 2400, about 2500, about 2600, about 2700, about 2800, about 2900, about 3000, about 3250, about 3500, about 3750 about 4000, about 4250, about 4500 or about 5000 ml/min.
clause 158. The method of any one of clauses 1-157 wherein, the polysaccharide containing solution is filtrated.
clause 159. The method of clause 158 wherein, said filtration is selected from the group consisting of depth filtration, filtration through activated carbon, size filtration, diafiltration and ultrafiltration.
clause 160. The method of clause 158 wherein, said filtration step is diafiltration.
clause 161. The method of clause 160 wherein, said filtration is tangential flow filtration.
clause 162. The method of clause 158 wherein, said filtration is depth filtration.
clause 163. The method of clause 162 wherein, wherein the depth filter design is selected from the group consisting of cassettes, cartridges, deep bed (e.g. sand filter) and lenticular filters.
clause 164. The method of any one of clauses 158-159 or 162-163 wherein the depth filter has a nominal retention range of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.
clause 165. The method of any one of clauses 158-159 or 162-163 wherein the depth filter has a nominal retention range of between about 0.01-75 micron, about 0.05-75 micron, about 0.1-75 micron, about 0.2-75 micron, about 0.3-75 micron, about 0.4-75 micron, about 0.5-75 micron, about 0.6-75 micron, about 0.7-75 micron, about 0.8-75 micron, about 0.9-75 micron, about 1-75 micron, about 1.25-75 micron, about 1.5-75 micron, about 1.75-75 micron, about 2-75 micron, about 3-75 micron, about 4-75 micron, about 5-75 micron, about 6-75 micron, about 7-75 micron, about 8-75 micron, about 9-75 micron, about 10-75 micron, about 15-75 micron, about 20-75 micron, about 25-75 micron, about 30-75 micron, about 40-75 micron or about 50-75 micron.
clause 166. The method of any one of clauses 158-159 or 162-163 wherein the depth filter has a nominal retention range of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.
clause 167. The method of any one of clauses 158-159 or 162-163 wherein the depth filter has a nominal retention range of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.
clause 168. The method of any one of clauses 158-159 or 162-163 wherein the depth filter has a nominal retention range of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.
clause 169. The method of any one of clauses 158-159 or 162-163 wherein the depth filter has a nominal retention range of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.
clause 170. The method of any one of clauses 158-159 or 162-163 wherein the depth filter has a nominal retention range of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.
clause 171. The method of any one of clauses 158-159 or 162-163 wherein the depth filter has a nominal retention range of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.
clause 172. The method of any one of clauses 158-159 or 162-163 wherein the depth filter has a nominal retention range of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.
clause 173. The method of any one of clauses 158-159 or 162-163 wherein the depth filter has a nominal retention range of between about between about 0.05-50 micron, 0.1-25 micron 0.2-10, micron 0.1-10 micron, 0.2-5 micron or 0.25-1 micron.
clause 174. The method of any one of clauses 158-159 or 162-173 wherein the depth filter has a filter capacity of 1-2500 L/m², 5-2500 L/m², 10-2500 L/m², 25-2500 L/m², 50-2500 L/m², 75-2500 L/m², 100-2500 L/m², 150-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².
clause 175. The method of any one of clauses 158-159 or 162-173 wherein the depth filter has a filter capacity of 1-1000 L/m², 5-1000 L/m², 10-1000 L/m², 25-1000 L/m², 50-1000 L/m², 75-1000 L/m², 100-1000 L/m², 150-1000 L/m², 200-1000 L/m², 300-1000 L/m², 400-1000 L/m², 500-1000 L/m² or 750-1000 L/m².
clause 176. The method of any one of clauses 155-156 or 159-170 wherein the depth filter has a filter capacity of 1-750 L/m², 5-750 L/m², 10-750 L/m², 25-750 L/m², 50-750 L/m², 75-750 L/m², 100-750 L/m², 150-750 L/m², 200-750 L/m², 300-750 L/m², 400-750 L/m² or 500-750 L/m².
clause 177. The method of any one of clauses 158-159 or 162-173 wherein the depth filter has a filter capacity of 1-500 L/m², 5-500 L/m², 10-500 L/m², 25-500 L/m², 50-500 L/m², 75-500 L/m², 100-500 L/m², 150-500 L/m², 200-500 L/m², 300-500 L/m² or 400-500 L/m².
clause 178. The method of any one of clauses 158-159 or 162-173 wherein the depth filter has a filter capacity of 1-400 L/m², 5-400 L/m², 10-400 L/m², 25-400 L/m², 50-400 L/m², 75-400 L/m², 100-400 L/m², 150-400 L/m², 200-400 L/m² or 300-400 L/m².
clause 179. The method of any one of clauses 158-159 or 162-173 wherein the depth filter has a filter capacity of 1-300 L/m², 5-300 L/m², 10-300 L/m², 25-300 L/m², 50-300 L/m², 75-300 L/m², 100-300 L/m², 150-300 L/m² or 200-300 L/m².
clause 180. The method of any one of clauses 158-159 or 162-173 wherein the depth filter has a filter capacity of 1-200 L/m², 5-200 L/m², 10-200 L/m², 25-200 L/m², 50-200 L/m², 75-200 L/m², 100-200 L/m² or 150-200 L/m².
clause 181. The method of any one of clauses 158-159 or 162-173 wherein the depth filter has a filter capacity of 1-100 L/m², 5-100 L/m², 10-100 L/m², 25-100 L/m², 50-100 L/m² or 75-100 L/m².
clause 182. The method of any one of clauses 158-159 or 162-173 wherein the depth filter has a filter capacity of 1-50 L/m², 5-50 L/m², 10-50 L/m² or 25-50 L/m².
clause 183. The method of any one of clauses 158-159 or 162-182 wherein the feed rate is between 1-1000 LMH (liters/m²/hour), 10-1000 LMH, 25-1000 LMH, 50-1000 LMH, 100-1000 LMH, 125-1000 LMH, 150-1000 LMH, 200-1000 LMH, 250-1000 LMH, 300-1000 LMH, 400-1000 LMH, 500-1000 LMH, 600-1000 LMH, 700-1000 LMH, 800-1000 LMH or 900-1000 LMH.
clause 184. The method of any one of clauses 158-159 or 162-182 wherein the feed rate is between 1-500 LMH, 10-500 LMH, 25-500 LMH, 50-500 LMH, 100-500 LMH, 125-500 LMH, 150-500 LMH, 200-500 LMH, 250-500 LMH, 300-500 LMH or 400-500 LMH.
clause 185. The method of any one of clauses 158-159 or 162-182 wherein the feed rate is between 1-400 LMH, 10-400 LMH, 25-400 LMH, 50-400 LMH, 100-400 LMH, 125-400 LMH, 150-400 LMH, 200-400 LMH, 250-400 LMH or 300-400 LMH.
clause 186. The method of any one of clauses 158-159 or 162-182 wherein the feed rate is between 1-250 LMH, 10-250 LMH, 25-250 LMH, 50-250 LMH, 100-250 LMH, 125-250 LMH, 150-250 LMH or 200-250 LMH.
clause 187. The method of any one of clauses 158-159 or 162-182 wherein the feed rate is about 1, about 2, about 5, about 10, about 25, about 50, about 60, about 70, about 80, about 90, about 100, about 110, about 120, about 130, about 140, about 150, about 160, about 170, about 180, about 190, about 200, about 210, about 220, about 230, about 240 about 250, about 260, about 270, about 280, about 290, about 300, about 310, about 320, about 330, about 340, about 350, about 360, about 370, about 380, about 390, about 400, about 425, about 450, about 475, about 500, about 525, about 550, about 575, about 600, about 650, about 700, about 750, about 800, about 850, about 900, about 950 or about 1000 LMH.
clause 188. The method of any one of clauses 158-187 wherein the filtrate is subjected to microfiltration.
clause 189. The method of clause 188 wherein the said microfiltration is dead-end filtration.
clause 190. The method of clause 188 wherein the said microfiltration is tangential microfiltration.
clause 191. The method of any one of clauses 188-190 wherein the microfiltration filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.
clause 192. The method of any one of clauses 188-190 wherein the microfiltration filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.
clause 193. The method of any one of clauses 188-190 wherein the microfiltration filter has a nominal retention range of about 0.01, about 0.05, about 0.1, about 0.2, about 0.3, about 0.4, about 0.45, about 0.5, about 0.6, about 0.7, about 0.8, about 0.9, about 1, about 1.1, about 1.2, about 1.3, about 1.4, about 1.5, about 1.6, about 1.7, about 1.8, about 1.9 or about 2 micron.
clause 194. The method of any one of clauses 188-190 wherein the microfiltration filter has a nominal retention of about 0.45 micron.
clause 195. The method of any one of clauses 188-194 wherein the microfiltration filter has a filter capacity of between 100-5000 L/m², 200-5000 L/m², 300-5000 L/m², 400-5000 L/m², 500-5000 L/m², 750-5000 L/m², 1000-5000 L/m², 1500-5000 L/m², 2000-5000 L/m², 3000-5000 L/m² or 4000-5000 L/m².
clause 196. The method of any one of clauses 188-194 wherein the microfiltration filter has a filter capacity of between 100-2500 L/m², 200-2500 L/m², 300-2500 L/m², 400-2500 L/m², 500-2500 L/m², 750-2500 L/m², 1000-2500 L/m², 1500-2500 L/m² or 2000-2500 L/m².
clause 197. The method of any one of clauses 188-194 wherein the microfiltration filter has a filter capacity of between 100-1500 L/m², 200-1500 L/m², 300-1500 L/m², 400-1500 L/m², 500-1500 L/m², 750-1500 L/m² or 1000-1500 L/m².
clause 198. The method of any one of clauses 188-194 wherein the microfiltration filter has a filter capacity of between 100-1250 L/m², 200-1250 L/m², 300-1250 L/m², 400-1250 L/m², 500-1250 L/m², 750-1250 L/m² or 1000-1250 L/m².
clause 199. The method of any one of clauses 188-194 wherein the microfiltration filter has a filter capacity of between 100-1000 L/m², 200-1000 L/m², 300-1000 L/m², 400-1000 L/m², 500-1000 L/m² or 750-1000 L/m².
clause 200. The method of any one of clauses 188-194 wherein the microfiltration filter has a filter capacity of between 100-750 L/m², 200-750 L/m², 300-750 L/m², 400-750 L/m² or 500-750 L/m².
clause 201. The method of any one of clauses 188-194 wherein the microfiltration filter has a filter capacity of between 100-600 L/m², 200-600 L/m², 300-600 L/m², 400-600 L/m² or 400-600 L/m².
clause 202. The method of any one of clauses 188-194 wherein the microfiltration filter has a filter capacity of between 100-500 L/m², 200-500 L/m², 300-500 L/m² or 400-500 L/m².
clause 203. The method of any one of clauses 188-194 wherein the microfiltration filter has a filter capacity of 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450 or about 2500 L/m2.
clause 204. The method of any one of clauses 158-203 wherein the filtrate is further treated by Ultrafiltration and/or Dialfiltration.
clause 205. The method of any one of clauses 158-203 wherein the filtrate is further treated by ultrafiltration.
clause 206. The method of any one of clauses 204-205 wherein the molecular weight cut off of the ultrafiltration membrane is in the range of between about 5 kDa-1000 kDa.
clause 207. The method of any one of clauses 204-205 wherein the molecular weight cut off of the ultrafiltration membrane is in the range of between about 10 kDa-750 kDa.
clause 208. The method of any one of clauses 204-205 wherein the molecular weight cut off of the ultrafiltration membrane is in the range of between about 10 kDa-500 kDa.
clause 209. The method of any one of clauses 204-205 wherein the molecular weight cut off of the ultrafiltration membrane is in the range of between about 10 kDa-300 kDa.
clause 210. The method of any one of clauses 204-205 wherein the molecular weight cut off of the ultrafiltration membrane is in the range of between about 10 kDa-100 kDa.
clause 211. The method of any one of clauses 204-205 wherein the molecular weight cut off of the ultrafiltration membrane is in the range of between about 10 kDa-50 kDa.
clause 212. The method of any one of clauses 204-205 wherein the molecular weight cut off of the ultrafiltration membrane is in the range of between about 10 kDa-30 kDa.
clause 213. The method of any one of clauses 204-205 wherein the molecular weight cut off of the ultrafiltration membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.
clause 214. The method of any one of clauses 204-205 wherein the molecular weight cut off of the ultrafiltration membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.
clause 215. The method of any one of clauses 204-205 wherein the molecular 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.
clause 216. The method of any one of clauses 204-205 wherein the molecular weight cut off of the ultrafiltration membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.
clause 217. The method of any one of clauses 204-205 wherein the molecular weight cut off of the ultrafiltration membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.
clause 218. The method of any one of clauses 204-217 wherein the concentration factor of the ultrafiltration step is from about 1.5 to about 10.
clause 219. The method of any one of clauses 204-217 wherein the concentration factor is from about 2 to about 8.
clause 220. The method of any one of clauses 204-217 wherein the concentration factor is from about 2 to about 5.
clause 221. The method of any one of clauses 204-217 wherein the concentration factor is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0.
clause 222. The method of any one of clauses 204-217 wherein the concentration factor is about 2, about 3, about 4, about 5, or about 6.
clause 223. The method of any one of clauses 204-222 wherein said ultrafiltration step is performed at temperature between about 20° C. to about 90° C.
clause 224. The method of any one of clauses 204-222 wherein said ultrafiltration step is performed at temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 225. The method of any one of clauses 204-222 wherein said ultrafiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 226. The method of any one of clauses 204-222 wherein said ultrafiltration step is performed at temperature of about 50° C.
clause 227. The method of any one of clauses 158-226 wherein the ultrafiltration filtrate is treated by diafiltration.
clause 228. The method of clause 227 wherein the replacement solution is water.
clause 229. The method of clause 227 wherein the replacement solution is saline in water.
clause 230. The method of clause 229 wherein the salt is selected from the group consisting of magnesium chloride, potassium chloride, sodium chloride and a combination thereof.
clause 231. The method of clause 229 wherein the salt is sodium chloride.
clause 232. The method of clause 229 wherein the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM.
clause 233. The method of clause 227 wherein the replacement solution is a buffer solution.
clause 234. The method of clause 227 wherein the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).
clause 235. The method of clause 227 wherein the replacement solution is a buffer solution wherein the buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate).
clause 236. The method of clause 227 wherein the replacement solution is a buffer solution wherein the buffer is a salt of citric acid (citrate).
clause 237. The method of clause 227 wherein the replacement solution is a buffer solution wherein the buffer is a salt of succinic acid (Succinate).
clause 238. The method of any one of clauses 234-237 said salt is a sodium salt.
clause 239. The method of any one of clauses 234-237 said salt is a potassium salt.
clause 240. The method of any one of clauses 233-239 wherein the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5.
clause 241. The method of any one of clauses 233-239 wherein the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0.
clause 242. The method of any one of clauses 233-239 wherein the pH of the diafiltration buffer is about 6.0, about 6.5, about 7.0, about 7.5, about 8.0, about 8.5 or about 9.0.
clause 243. The method of any one of clauses 226-231 wherein the pH of the diafiltration buffer is about 6.5, about 7.0 or about 7.5.
clause 244. The method of any one of clauses 233-239 wherein the pH of the diafiltration buffer is about 7.0.
clause 245. The method of any one of clauses 233-244 wherein the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.
clause 246. The method of any one of clauses 233-244 wherein the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.
clause 247. The method of any one of clauses 233-244 wherein the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.
clause 248. The method of any one of clauses 233-244 wherein the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.
clause 249. The method of any one of clauses 233-244 wherein the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.
clause 250. The method of any one of clauses 233-244 wherein the concentration of the diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
clause 251. The method of any one of clauses 233-244 wherein the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.
clause 252. The method of any one of clauses 233-244 wherein the concentration of the diafiltration buffer is about 10 mM.
clause 253. The method of any one of clauses 233-252 wherein the replacement solution comprises a chelating agent.
clause 254. The method of any one of clauses 233-252 wherein the replacement solution comprises an alum chelating agent.
clause 255. The method of any one of clauses 233-252 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.
clause 256. The method of any one of clauses 233-255 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), a salt of citric acid (citrate) and combinations of these.
clause 257. The method of any one of clauses 233-254 wherein the replacement solution comprises Ethylene Diamine Tetra Acetate (EDTA) as chelating agent.
clause 258. The method of any one of clauses 233-254 wherein the replacement solution comprises a salt of citric acid (citrate) as chelating agent.
clause 259. The method of any one of clauses 233-254 wherein the replacement solution comprises sodium citrate as chelating agent.
clause 260. The method of any one of clauses 253-258 wherein the concentration of the chelating agent in the replacement solution is from 1 to 500 mM.
clause 261. The method of any one of clauses 253-258 wherein the concentration of the chelating agent in the replacement solution is from 2 to 400 mM.
clause 262. The method of any one of clauses 253-258 wherein concentration of the chelating agent in the replacement solution is from 10 to 400 mM.
clause 263. The method of any one of clauses 253-258 wherein concentration of the chelating agent in the replacement solution is from 10 to 200 mM.
clause 264. The method of any one of clauses 253-258 wherein concentration of the chelating agent in the replacement solution is from 10 to 100 mM.
clause 265. The method of any one of clauses 253-258 wherein concentration of the chelating agent in the replacement solution is from 10 to 50 mM.
clause 266. The method of any one of clauses 253-258 wherein concentration of the chelating agent in the replacement solution is from 10 to 30 mM.
clause 267. The method of any one of clauses 253-258 wherein concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, 30 about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
clause 268. The method of any one of clauses 253-258 wherein concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.
clause 269. The method of any one of clauses 253-258 wherein concentration of the chelating agent in the replacement solution is about 15 mM, about 20 mM, about 25 mM, about 30 mM, about 35 mM, about 40 mM, about 45 mM or about 50 mM.
clause 270. The method of any one of clauses 233-269 wherein the replacement solution comprises a salt.
clause 271. The method of clause 270 wherein, the salt is selected from the groups consisting of magnesium chloride, potassium chloride, sodium chloride and a combination thereof.
clause 272. The method of clause 270 wherein, the salt is sodium chloride.
clause 273. The method of any one of clauses 270-272 wherein the replacement solution comprises sodium chloride at 1 about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.
clause 274. The method of any one of clauses 227-273 wherein the number of diavolumes is at least 5, 10, 15, 20, 25, 30, 35, 40, 45, or 50.
clause 275. The method of any one of clauses 227-273 wherein the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100.
clause 276. The method of any one of clauses 227-273 wherein the number of diavolumes is about 5, about 6, about 7, about 8, about 9, about 10, about 11, about 12, about 13, about 14 or about 15.
clause 277. The method of any one of clauses 227-276 wherein said dialfiltration step is performed at temperature of between about 20° C. to about 90° C.
clause 278. The method of any one of clauses 227-276 wherein said dialfiltration step is performed at temperature of between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 279. The method of any one of clauses 227-276 wherein said dialfiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 280. The method of any one of clauses 227-276 wherein said dialfiltration step is performed at temperature of about 50° C.
clause 281. The method of any one of clauses 204-277 wherein said ultrafiltration and dialfiltration steps if both conducted are performed at a temperature between about 20° C. to about 90° C.
clause 282. The method of any one of clauses 204-277 wherein said ultrafiltration and dialfiltration steps if both conducted are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 283. The method of any one of clauses 204-277 wherein said ultrafiltration and dialfiltration steps if both conducted are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 284. The method of any one of clauses 204-277 wherein said ultrafiltration and dialfiltration steps if both conducted are performed at a temperature of about 50° C.
clause 285. The method of any one of clauses 1-284 wherein the solution containing the polysaccharide (e.g. the supernatant, the filtrate or retentate) is treated by an activated carbon filtration step.
clause 286. The method of any one of clause 285 wherein the activated carbon is added in the form of a powder, as a granular carbon bed, as a pressed carbon block or extruded carbon block (see e.g. Norit active charcoal).
clause 287. The method of clause 286 wherein the activated carbon is added in an amount of about 0.1 to 20% (weight volume), 1 to 15% (weight volume), 1 to 10% (weight volume), 2 to 10% (weight volume), 3 to 10% (weight volume), 4 to 10% (weight volume), 5 to 10% (weight volume), 1 to 5% (weight volume) or 2 to 5% (weight volume).
clause 288. The method of any one of clause 286-287 wherein the mixture is stirred and left to stand.
clause 289. The method of any one of clause 286-287 wherein the mixture is stirred and left to stand for about 5, about 10, about 15, about 20, about 30, about 45, about 60, about 90, about 120, about 180, about 240 minutes or more.
clause 290. The method of any one of clause 286-289 wherein the activated carbon is then removed.
clause 291. The method of any one of clause 286-290 wherein the activated carbon is removed by centrifugation or filtration.
clause 292. The method of clause 285 wherein the solution is filtered through activated carbon immobilized in a matrix.
clause 293. The method of clause 285 wherein said matrix is a porous filter medium permeable for the solution.
clause 294. The method of any one of clauses 292-293 wherein said matrix comprises a support material.
clause 295. The method of any one of clauses 292-293 wherein said matrix comprises a binder material.
clause 296. The method of any one of clauses 294-295 wherein said support material is a synthetic polymer.
clause 297. The method of any one of clauses 294-295 wherein said support material is a polymer of natural origin.
clause 298. The method of clause 296 wherein said synthetic polymers includes any one of polystyrene, polyacrylamide or polymethyl methacrylate.
clause 299. The method of clause 296 wherein said synthetic polymers is selected from the group consisting of polystyrene, polyacrylamide and polymethyl methacrylate.
clause 300. The method of clause 297 wherein said a polymer of natural origin include includes any one of cellulose, polysaccharide, dextran or agarose.
clause 301. The method of clause 297 wherein said a polymer of natural is selected from the group consisting of cellulose, polysaccharide, dextran and agarose.
clause 302. The method of any one of clauses 294-301 wherein said polymer support material if present is in the form of a fibre network to provide mechanical rigidity.
clause 303. The method of any one of clauses 294-302 wherein said binder material if present is a resin.
clause 304. The method of any one of clauses 292-303 wherein said matrix has the form of a membrane sheet.
clause 305. The method of any one of clauses 292-304 wherein the activated carbon immobilized in the matrix is in the form of a flow-through carbon cartridge.
clause 306. The method of any one of clauses 304 wherein the membrane sheet is spirally wound.
clause 307. The method of any one of clauses 292-306, wherein several discs are stacked upon each other.
clause 308. The method of clauses 307 wherein the configuration of stacked discs is lenticular.
clause 309. The method of any one of clauses 292-308, wherein the activated carbon in the carbon filter is derived from peat, lignite, wood or coconut shell.
clause 310. The method of any one of clauses 292-309, wherein the activated carbon immobilized in a matrix is placed in a housing to form an independent filter unit.
clause 311. The method of any one of clauses 292-310, wherein the activated carbon filters comprise a cellulose matrix into which activated carbon powder is entrapped and resin-bonded in place.
clause 312. The method of any one of clauses 285-311, wherein the activated carbon filter has a nominal micron rating of between about 0.01-100 micron, about 0.05-100 micron, about 0.1-100 micron, about 0.2-100 micron, about 0.3-100 micron, about 0.4-100 micron, about 0.5-100 micron, about 0.6-100 micron, about 0.7-100 micron, about 0.8-100 micron, about 0.9-100 micron, about 1-100 micron, about 1.25-100 micron, about 1.5-100 micron, about 1.75-100 micron, about 2-100 micron, about 3-100 micron, about 4-100 micron, about 5-100 micron, about 6-100 micron, about 7-100 micron, about 8-100 micron, about 9-100 micron, about 10-100 micron, about 15-100 micron, about 20-100 micron, about 25-100 micron, about 30-100 micron, about 40-100 micron, about 50-100 micron or about 75-100 micron.
clause 313. The method of any one of clauses 285-311, wherein the activated carbon filter has a nominal micron rating of between about 0.01-50 micron, about 0.05-50 micron, about 0.1-50 micron, about 0.2-50 micron, about 0.3-50 micron, about 0.4-50 micron, about 0.5-50 micron, about 0.6-50 micron, about 0.7-50 micron, about 0.8-50 micron, about 0.9-50 micron, about 1-50 micron, about 1.25-50 micron, about 1.5-50 micron, about 1.75-50 micron, about 2-50 micron, about 3-50 micron, about 4-50 micron, about 5-50 micron, about 6-50 micron, about 7-50 micron, about 8-50 micron, about 9-50 micron, about 10-50 micron, about 15-50 micron, about 20-50 micron, about 25-50 micron, about 30-50 micron, about 40-50 micron or about 50-50 micron.
clause 314. The method of any one of clauses 285-311, wherein the activated carbon filter has a nominal micron rating of between about 0.01-25 micron, about 0.05-25 micron, about 0.1-25 micron, about 0.2-25 micron, about 0.3-25 micron, about 0.4-25 micron, about 0.5-25 micron, about 0.6-25 micron, about 0.7-25 micron, about 0.8-25 micron, about 0.9-25 micron, about 1-25 micron, about 1.25-25 micron, about 1.5-25 micron, about 1.75-25 micron, about 2-25 micron, about 3-25 micron, about 4-25 micron, about 5-25 micron, about 6-25 micron, about 7-25 micron, about 8-25 micron, about 9-25 micron, about 10-25 micron, about 15-25 micron or about 20-25 micron.
clause 315. The method of any one of clauses 285-311, wherein the activated carbon filter has a nominal micron rating of between about 0.01-10 micron, about 0.05-10 micron, about 0.1-10 micron, about 0.2-10 micron, about 0.3-10 micron, about 0.4-10 micron, about 0.5-10 micron, about 0.6-10 micron, about 0.7-10 micron, about 0.8-10 micron, about 0.9-10 micron, about 1-10 micron, about 1.25-10 micron, about 1.5-10 micron, about 1.75-10 micron, about 2-10 micron, about 3-10 micron, about 4-10 micron, about 5-10 micron, about 6-10 micron, about 7-10 micron, about 8-10 micron or about 9-10 micron.
clause 316. The method of any one of clauses 285-311, wherein the activated carbon filter has a nominal micron rating of between about 0.01-8 micron, about 0.05-8 micron, about 0.1-8 micron, about 0.2-8 micron, about 0.3-8 micron, about 0.4-8 micron, about 0.5-8 micron, about 0.6-8 micron, about 0.7-8 micron, about 0.8-8 micron, about 0.9-8 micron, about 1-8 micron, about 1.25-8 micron, about 1.5-8 micron, about 1.75-8 micron, about 2-8 micron, about 3-8 micron, about 4-8 micron, about 5-8 micron, about 6-8 micron or about 7-8 micron.
clause 317. The method of any one of clauses 285-311, wherein the activated carbon filter has a nominal micron rating of between about 0.01-5 micron, about 0.05-5 micron, about 0.1-5 micron, about 0.2-5 micron, about 0.3-5 micron, about 0.4-5 micron, about 0.5-5 micron, about 0.6-5 micron, about 0.7-5 micron, about 0.8-5 micron, about 0.9-5 micron, about 1-5 micron, about 1.25-5 micron, about 1.5-5 micron, about 1.75-5 micron, about 2-5 micron, about 3-5 micron or about 4-5 micron.
clause 318. The method of any one of clauses 285-311, wherein the activated carbon filter has a nominal micron rating of between about 0.01-2 micron, about 0.05-2 micron, about 0.1-2 micron, about 0.2-2 micron, about 0.3-2 micron, about 0.4-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, about 1.75-2 micron, about 2-2 micron, about 3-2 micron or about 4-2 micron.
clause 319. The method of any one of clauses 285-311, wherein the activated carbon filter has a nominal micron rating of between about 0.01-1 micron, about 0.05-1 micron, about 0.1-1 micron, about 0.2-1 micron, about 0.3-1 micron, about 0.4-1 micron, about 0.5-1 micron, about 0.6-1 micron, about 0.7-1 micron, about 0.8-1 micron or about 0.9-1 micron.
clause 320. The method of any one of clauses 285-311, wherein the activated carbon filter has a nominal micron rating of between about 0.05-50 micron, 0.1-25 micron 0.2-10, micron 0.1-10 micron, 0.2-5 micron or 0.25-1 micron.
clause 321. The method of any one of clauses 285-320, wherein the activated carbon filter is conducted at a feed rate of between 1-500 LMH, 10-500 LMH, 15-500 LMH, 20-500 LMH, 25-500 LMH, 30-500 LMH, 40-500 LMH, 50-500 LMH, 100-500 LMH, 125-500 LMH, 150-500 LMH, 200-500 LMH, 250-500 LMH, 300-500 LMH or 400-500 LMH.
clause 322. The method of any one of clauses 285-320, wherein the activated carbon filter is conducted at a feed rate of between 1-200 LMH, 10-200 LMH, 15-200 LMH, 20-200 LMH, 25-200 LMH, 30-200 LMH, 40-200 LMH, 50-200 LMH, 100-200 LMH, 125-200 LMH or 150-200 LMH.
clause 323. The method of any one of clauses 285-320, wherein the activated carbon filter is conducted at a feed rate of between 1-150 LMH, 10-150 LMH, 15-150 LMH, 20-150 LMH, 25-150 LMH, 30-150 LMH, 40-150 LMH, 50-150 LMH, 100-150 LMH or 125-150 LMH.
clause 324. The method of any one of clauses 285-320, wherein the activated carbon filter is conducted at a feed rate of between 1-100 LMH, 10-100 LMH, 15-100 LMH, 20-100 LMH, 25-100 LMH, 30-100 LMH, 40-100 LMH, or 50-100 LMH.
clause 325. The method of any one of clauses 285-320, wherein the activated carbon filter is conducted at a feed rate of between 1-75 LMH, 5-75 LMH, 10-75 LMH, 15-75 LMH, 20-75 LMH, 25-75 LMH, 30-75 LMH, 35-75 LMH, 40-75 LMH, 45-75 LMH, 50-75 LMH, 55-75 LMH, 60-75 LMH, 65-75 LMH, or 70-75 LMH.
clause 326. The method of any one of clauses 285-320, wherein the activated carbon filter is conducted at a feed rate of between 1-50 LMH, 5-50 LMH, 7-50 LMH, 10-50 LMH, 15-50 LMH, 20-50 LMH, 25-50 LMH, 30-50 LMH, 35-50 LMH, 40-50 LMH or 45-50 LMH.
clause 327. The method of any one of clauses 285-320, wherein the activated carbon filter is conducted at a feed rate of about 1, about 2, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 300, about 350, about 400, about 450, about 500, about 550, about 600, about 700, about 800, about 900, about 950 or about 1000 LMH.
clause 328. The method of any one of clauses 285-327, wherein the solution is treated by an activated carbon filter wherein the filter has a filter capacity of between 5-1000 L/m², 10-750 L/m², 15-500 L/m², 20-400 L/m², 25-300 L/m², 30-250 L/m², 40-200 L/m² or 30-100 L/m².
clause 329. The method of any one of clauses 285-327, wherein the solution is treated by an activated carbon filter wherein the filter has a filter capacity of about 5, about 10, about 15, about 20, about 25, about 30, about 35, about 40, about 45, about 50, about 55, about 60, about 65, about 70, about 75, about 80, about 85, about 90, about 100, about 125, about 150, about 175, about 200, about 225, about 250, about 275, about 300, about 400, about 500, about 600, about 700, about 800, about 900, or about 1000 L/m².
clause 330. The method of any one of clauses 285-329, wherein 1, 2, 3, 4, 5, 6, 7, 8, 9, or 10 activated carbon filtration step(s) are performed.
clause 331. The method of any one of clauses 285-329, wherein 1, 2 or 3 activated carbon filtration step(s) are performed.
clause 332. The method of any one of clauses 285-329, wherein 1 or 2 activated carbon filtration step(s) are performed.
clause 333. The method of any one of clauses 285-332, wherein the solution is treated by activated carbon filters in series.
clause 334. The method of any one of clauses 285-332, wherein the solution is treated by 1, 2, 3, 4, 5, 6, 7, 8, 9, or 10 activated carbon filters in series.
clause 335. The method of any one of clauses 285-332, wherein the solution is treated by 2, 3, 4 or 5 activated carbon filters in series.
clause 336. The method of any one of clauses 285-332, wherein the solution is treated by 2 activated carbon filters in series.
clause 337. The method of any one of clauses 285-332, wherein the solution is treated by 3 activated carbon filters in series.
clause 338. The method of any one of clauses 285-332, wherein the solution is treated by 4 activated carbon filters in series.
clause 339. The method of any one of clauses 285-332, wherein the solution is treated by 5 activated carbon filters in series.
clause 340. The method of any one of clauses 285-339, wherein the activated carbon filtration step is performed in a single pass mode.
clause 341. The method of any one of clauses 285-339, wherein the activated carbon filtration step is performed in recirculation mode.
clause 342. The method of clause 341, wherein 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles of activated carbon filtration are performed.
clause 343. The method of clause 341, wherein 2, 3, 4, 5, 6, 7, 8, 9 or 10 cycles of activated carbon filtration are performed.
clause 344. The method of clause 341, wherein 2 or 3 cycles of activated carbon filtration are performed.
clause 345. The method of clause 341, wherein 2 cycles of activated carbon filtration are performed.
clause 346. The method of any one of clauses 285-345, wherein the filtrate is further filtered.
clause 347. The method of any one of clauses 285-345, wherein the filtrate is subjected to microfiltration.
clause 348. The method of clause 347, wherein said microfiltration is dead-end filtration (perpendicular filtration).
clause 349. The method of clause 347, wherein said microfiltration is tangential microfiltration.
clause 350. The method of any one of clauses 347-349, wherein said microfiltration filter has a nominal retention range of between about 0.01-2 micron, about 0.05-2 micron, about 0.1-2 micron, about 0.2-2 micron, about 0.3-2 micron, about 0.4-2 micron, about 0.45-2 micron, about 0.5-2 micron, about 0.6-2 micron, about 0.7-2 micron, about 0.8-2 micron, about 0.9-2 micron, about 1-2 micron, about 1.25-2 micron, about 1.5-2 micron, or about 1.75-2 micron.
clause 351. The method of any one of clauses 347-349, wherein said microfiltration filter has a nominal retention range of between about 0.01-1 micron, about 0.05-1 micron, about 0.1-1 micron, about 0.2-1 micron, about 0.3-1 micron, about 0.4-1 micron, about 0.45-1 micron, about 0.5-1 micron, about 0.6-1 micron, about 0.7-1 micron, about 0.8-1 micron or about 0.9-1 micron.
clause 352. he method of any one of clauses 347-349, wherein said microfiltration filter has a nominal retention range of about 0.01, about 0.05, about 0.1, about 0.2, about 0.3, about 0.4, about 0.45, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9 or about 2.0 micron.
clause 353. The method of any one of clauses 343-345, wherein said microfiltration filter has a nominal retention range of about 0.2 micron.
clause 354. The method of any one of clauses 347-353, wherein said microfiltration filter has a filter capacity of 100-6000 L/m², 200-6000 L/m², 300-6000 L/m², 400-6000 L/m², 500-6000 L/m², 750-6000 L/m², 1000-6000 L/m², 1500-6000 L/m², 2000-6000 L/m², 3000-6000 L/m² or 4000-6000 L/m².
clause 355. The method of any one of clauses 347-353, wherein said microfiltration filter has a filter capacity of 100-4000 L/m², 200-4000 L/m², 300-4000 L/m², 400-4000 L/m², 500-4000 L/m², 750-4000 L/m², 1000-4000 L/m², 1500-4000 L/m², 2000-4000 L/m², 2500-4000 L/m², 3000-4000 L/m², 3000-4000 L/m² or 3500-4000 L/m².
clause 356. The method of any one of clauses 347-353, wherein said microfiltration filter has a filter capacity of 100-3750 L/m², 200-3750 L/m², 300-3750 L/m², 400-3750 L/m², 500-3750 L/m², 750-3750 L/m², 1000-3750 L/m², 1500-3750 L/m², 2000-3750 L/m², 2500-3750 L/m², 3000-3750 L/m², 3000-3750 L/m² or 3500-3750 L/m².
clause 357. The method of any one of clauses 347-353, wherein said microfiltration filter has a filter capacity of 100-1250 L/m², 200-1250 L/m², 300-1250 L/m², 400-1250 L/m², 500-1250 L/m², 750-1250 L/m² or 1000-1250 L/m².
clause 358. The method of any one of clauses 347-353, wherein said microfiltration filter has a filter capacity of about 100, about 200, about 300, about 400, about 550, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5250, about 5500, about 5750 or about 6000 L/m².
clause 359. The method of any of clauses 1 to 358, wherein the polysaccharide-containing solution or filtrate is further purified by hydrophobic interaction chromatography.
clause 360. The method of clause 359 wherein the hydrophobic interaction chromatography is conducted using an hydrophobic adsorbent selected from the group consisting of a phenyl membrane, butyl-agarose, phenyl-agarose, octyl-agarose, butyl organic polymer resin, phenyl organic polymer resin, ether organic polymer resin, polypropylenglycol organic polymer resin and hexyl organic polymer resin.
clause 361. The method of clause 360 wherein the hydrophobic interaction chromatography is conducted using a phenyl membrane.
clause 362. The method of clause 361 wherein the hydrophobic interaction chromatography is conducted using a Sartobind @ Phenyl membrane or a Cytiva @ Phenyl Adsorber membrane.
clause 363. The method of any of clauses 359 to 362, wherein the polysaccharide-containing solution or the filtrate is treated with an equilibration buffer comprising a salt to obtain a running buffer having a salt concentration selected from about 0.1, about 0.2, about 0.3, about 0.4, about 0.5, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9 or about 7.0M.
clause 364. The method of clause 363 wherein the pH of the running buffer is about 4.0 to about 8.0.
clause 365. The method of clause 364 wherein the pH of the running buffer is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9 or about 8.0.
clause 366. The method of clause 363 to 365, wherein the salt is selected from ammonium sulfate, sodium phosphate, potassium phosphate, sodium sulfate, sodium citrate or sodium chloride.
clause 367. The method of clause 363 wherein the running buffer comprises ammonium sulfate at a concentration comprised between about 0.5M and about 3.0M at pH 6.0±2.0.
clause 368. The method of clause 367 wherein the running buffer comprises ammonium sulfate at a concentration comprised between about 1.0M and 2.0M at a pH of about 6.0.
clause 369. The method of clause 363 wherein the running buffer comprises sodium phosphate at concentration comprised between about 0.5M and about 3.0M at pH 7.0±1.5.
clause 370. The method of clause 363 wherein the running buffer comprises potassium phosphate at concentration comprised between about 0.5M and about 3.0M at pH 7.0±1.5.
clause 371. The method of clause 363 wherein the running buffer comprises sodium sulfate at concentration comprised between about 0.1 M and about 0.75M at pH 6.0±2.0.
clause 372. The method of clause 363 wherein the running buffer comprises sodium citrate at concentration comprised between about 0.1M and about 1.5M at pH 6.0±2.0.
clause 373. The method of clause 363 wherein the running buffer comprises sodium chloride at concentration comprised between about 0.5M and about 5.0M at pH 7.0±1.5.
clause 374. The method of any of clauses 363 to 373 wherein the hydrophobic adsorbent is equilibrated with the running buffer.
clause 375. The method according to any one of clause 360 to 374 wherein the hydrophobic adsorbent is a phenyl membrane and the flow rate is comprised between about 0.1 and about 20 membrane volumes per min, about 0.1 and about 10 membrane volumes per min, about 0.2 and about 10 membrane volumes per min, about 0.2 and about 5 membrane volumes per min or about 0.1 and about 1 membrane volume per min.
clause 376. The method of clause 375 wherein the flow rate is comprised between about 0.1 and about 1.0 membrane volume per min.
clause 377. The method of claim 376 wherein the flow rate is about 0.1, about 0.2, about 0.3, about 0.4, about 0.5, about 0.6, about 0.7, about 0.8, about 0.9 or about 1.0 membrane volume per min.
clause 378. The method of any one of clauses 363 to 375 wherein the wherein the hydrophobic adsorbent is washed with the running buffer.
clause 379. The method of any one of clauses 285-378, wherein the filtrate is further clarified by ultrafiltration and/or dialfiltration.
clause 380. The method of any one of clauses 285-378, wherein the filtrate is further clarified by ultrafiltration.
clause 381. The methods of clause 379 or 380 wherein the molecular weight cut off of said ultrafiltration membrane is in the range of between about 5 kDa-1000 kDa.
clause 382. The methods of clause 379 or 380 wherein the molecular weight cut off of said ultrafiltration membrane is in the range of between about 10 kDa-750 kDa.
clause 383. The methods of clause 379 or 380 wherein the molecular weight cut off of said ultrafiltration membrane is in the range of between about 10 kDa-500 kDa.
clause 384. The methods of clause 379 or 380 wherein the molecular weight cut off of said ultrafiltration membrane is in the range of between about 10 kDa-300 kDa.
clause 385. The methods of clause 379 or 380 wherein the molecular weight cut off of said ultrafiltration membrane is in the range of between about 10 kDa-100 kDa.
clause 386. The methods of clause 379 or 380 wherein the molecular weight cut off of said ultrafiltration membrane is in the range of between about 10 kDa-50 kDa.
clause 387. The methods of clause 379 or 380 wherein the molecular weight cut off of said ultrafiltration membrane is in the range of between about 10 kDa-30 kDa.
clause 388. The methods of clause 379 or 380 wherein the molecular weight cut off of said ultrafiltration membrane is in the range of between about 5 kDa-1000 kDa, about 10 kDa-1000 kDa about 20 kDa-1000 kDa, about 30 kDa-1000 kDa, about 40 kDa-1000 kDa, about 50 kDa-1000 kDa, about 75 kDa-1000 kDa, about 100 kDa-1000 kDa, about 150 kDa-1000 kDa, about 200 kDa-1000 kDa, about 300 kDa-1000 kDa, about 400 kDa-1000 kDa, about 500 kDa-1000 kDa or about 750 kDa-1000 kDa.
clause 389. The methods of clause 379 or 380 wherein the molecular weight cut off of said ultrafiltration membrane is in the range of between about 5 kDa-500 kDa, about 10 kDa-500 kDa, about 20 kDa-500 kDa, about 30 kDa-500 kDa, about 40 kDa-500 kDa, about 50 kDa-500 kDa, about 75 kDa-500 kDa, about 100 kDa-500 kDa, about 150 kDa-500 kDa, about 200 kDa-500 kDa, about 300 kDa-500 kDa or about 400 kDa-500 kDa.
clause 390. The methods of clause 379 or 380 wherein the molecular weight cut off of said ultrafiltration membrane is in the range of between about 5 kDa-300 kDa, about 10 kDa-300 kDa, about 20 kDa-300 kDa, about 30 kDa-300 kDa, about 40 kDa-300 kDa, about 50 kDa-300 kDa, about 75 kDa-300 kDa, about 100 kDa-300 kDa, about 150 kDa-300 kDa or about 200 kDa-300 kDa.
clause 391. The methods of clause 379 or 380 wherein the molecular weight cut off of said ultrafiltration membrane is in the range of between about 5 kDa-100 kDa, about 10 kDa-100 kDa, about 20 kDa-100 kDa, about 30 kDa-100 kDa, about 40 kDa-100 kDa, about 50 kDa-100 kDa or about 75 kDa-100 kDa.
clause 392. The methods of clause 379 or 380 wherein the molecular weight cut off of said ultrafiltration membrane is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 250 kDa, about 300 kDa, about 400 kDa, about 500 kDa, about 750 kDa or about 1000 kDa.
clause 393. The methods of any one of clause 379-381 wherein the concentration factor of said ultrafiltration step is from about 1.5 to about 10.0.
clause 394. The methods of any one of clause 379-381 wherein the concentration factor of said ultrafiltration step is from about 2.0 to about 8.0.
clause 395. The methods of any one of clause 379-381 wherein the concentration factor of said ultrafiltration step is from about 2.0 to about 5.0.
clause 396. The methods of any one of clause 379-381 wherein the concentration factor of said ultrafiltration step is about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In an embodiment, the concentration factor is about 2.0, about 3.0, about 4.0, about 5.0, or about 6.0.
clause 397. The method of any one of clauses 379-396 wherein said ultrafiltration step is performed at temperature between about 20° C. to about 90° C.
clause 398. The method of any one of clauses 379-396 wherein said ultrafiltration step is performed at temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 399. The method of any one of clauses 379-396 wherein said ultrafiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 400. The method of any one of clauses 379-396 wherein said ultrafiltration step is performed at temperature of about 50° C.
clause 401. The method of any one of clauses 379-396 wherein the ultrafiltration filtrate is treated by diafiltration.
clause 402. The method of clauses 401 wherein the replacement solution is water.
clause 403. The method of clause 401 wherein the replacement solution is saline in water.
clause 404. The method of clause 403 wherein the salt is selected from the group consisting of magnesium chloride, potassium chloride, sodium chloride and a combination thereof.
clause 405. The method of clauses 403 wherein the salt is sodium chloride.
clause 406. The method of clauses 403 wherein the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM.
clause 407. The method of clause 401 wherein the replacement solution is a buffer solution.
clause 408. The method of clause 401 wherein the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris).
clause 409. The method of clause 401 wherein the replacement solution is a buffer solution wherein the buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate).
clause 410. The method of clause 401 wherein the replacement solution is a buffer solution wherein the buffer is a salt of citric acid (citrate).
clause 411. The method of clause 401 wherein the replacement solution is a buffer solution wherein the buffer is a salt of succinic acid (succinate).
clause 412. The method of clause 401 wherein the replacement solution is a buffer solution wherein the buffer is a salt of phosphoric acid (phosphate).
clause 413. The method of any one of clauses 408-412 said salt is a sodium salt.
clause 414. The method of any one of clauses 408-412 said salt is a potassium salt.
clause 415. The method of clause 401 wherein the replacement solution is a buffer solution wherein the buffer is potassium phosphate.
clause 416. The method of any one of clauses 401-415 wherein the pH of the diafiltration buffer is between about 4.0-11.0, between about 5.0-10.0, between about 5.5-9.0, between about 6.0-8.0, between about 6.0-7.0, between about 6.5-7.5, between about 6.5-7.0 or between about 6.0-7.5.
clause 417. The method of clause 401-415 wherein the pH of the diafiltration buffer is about 4.0, about 4.5, about 5.0, about 5.5, about 6.0, about 6.5, about 7.0, about 7.5, about 8.0, about 8.5, about 9.0, about 9.5, about 10.0, about 10.5 or about 11.0.
clause 418. The method of any one of clauses 401-415 wherein the pH of the diafiltration buffer is about 6.0, about 6.5, about 7.0, about 7.5, about 8.0, about 8.5 or about 9.0.
clause 419. The method of any one of clauses 401-415 wherein the pH of the diafiltration buffer is about 6.5, about 7.0 or about 7.5.
clause 420. The method of any one of clauses 401-415 wherein the pH of the diafiltration buffer is about 6.0.
clause 421. The method of any one of clauses 401-415 wherein the pH of the diafiltration buffer is about 6.5.
clause 422. The method of any one of clauses 401-415 wherein the pH of the diafiltration buffer is about 7.0
clause 423. The method of any one of clauses 407-422 wherein the concentration of the diafiltration buffer is between about 0.01 mM-100 mM, between about 0.1 mM-100 mM, between about 0.5 mM-100 mM, between about 1 mM-100 mM, between about 2 mM-100 mM, between about 3 mM-100 mM, between about 4 mM-100 mM, between about 5 mM-100 mM, between about 6 mM-100 mM, between about 7 mM-100 mM, between about 8 mM-100 mM, between about 9 mM-100 mM, between about 10 mM-100 mM, between about 11 mM-100 mM, between about 12 mM-100 mM, between about 13 mM-100 mM, between about 14 mM-100 mM, between about 15 mM-100 mM, between about 16 mM-100 mM, between about 17 mM-100 mM, between about 18 mM-100 mM, between about 19 mM-100 mM, between about 20 mM-100 mM, between about 25 mM-100 mM, between about 30 mM-100 mM, between about 35 mM-100 mM, between about 40 mM-100 mM, between about 45 mM-100 mM, between about 50 mM-100 mM, between about 55 mM-100 mM, between about 60 mM-100 mM, between about 65 mM-100 mM, between about 70 mM-100 mM, between about 75 mM-100 mM, between about 80 mM-100 mM, between about 85 mM-100 mM, between about 90 mM-100 mM or between about 95 mM-100 mM.
clause 424. The method of any one of clauses 407-422 wherein the concentration of the diafiltration buffer is between about 0.01 mM-50 mM, between about 0.1 mM-50 mM, between about 0.5 mM-50 mM, between about 1 mM-50 mM, between about 2 mM-50 mM, between about 3 mM-50 mM, between about 4 mM-50 mM, between about 5 mM-50 mM, between about 6 mM-50 mM, between about 7 mM-50 mM, between about 8 mM-50 mM, between about 9 mM-50 mM, between about 10 mM-50 mM, between about 11 mM-50 mM, between about 12 mM-50 mM, between about 13 mM-50 mM, between about 14 mM-50 mM, between about 15 mM-50 mM, between about 16 mM-50 mM, between about 17 mM-50 mM, between about 18 mM-50 mM, between about 19 mM-50 mM, between about 20 mM-50 mM, between about 25 mM-50 mM, between about 30 mM-50 mM, between about 35 mM-50 mM, between about 40 mM-50 mM or between about 45 mM-50 mM.
clause 425. The method of any one of clauses 407-422 wherein the concentration of the diafiltration buffer is between about 0.01 mM-25 mM, between about 0.1 mM-25 mM, between about 0.5 mM-25 mM, between about 1 mM-25 mM, between about 2 mM-25 mM, between about 3 mM-25 mM, between about 4 mM-25 mM, between about 5 mM-25 mM, between about 6 mM-25 mM, between about 7 mM-25 mM, between about 8 mM-25 mM, between about 9 mM-25 mM, between about 10 mM-25 mM, between about 11 mM-25 mM, between about 12 mM-25 mM, between about 13 mM-25 mM, between about 14 mM-25 mM, between about 15 mM-25 mM, between about 16 mM-25 mM, between about 17 mM-25 mM, between about 18 mM-25 mM, between about 19 mM-25 mM or between about 20 mM-25 mM.
clause 426. The method of any one of clauses 407-422 wherein the concentration of the diafiltration buffer is between about 0.01 mM-15 mM, between about 0.1 mM-15 mM, between about 0.5 mM-15 mM, between about 1 mM-15 mM, between about 2 mM-15 mM, between about 3 mM-15 mM, between about 4 mM-15 mM, between about 5 mM-15 mM, between about 6 mM-15 mM, between about 7 mM-15 mM, between about 8 mM-15 mM, between about 9 mM-15 mM, between about 10 mM-15 mM, between about 11 mM-15 mM, between about 12 mM-15 mM, between about 13 mM-15 mM or between about 14 mM-15 mM.
clause 427. The method of any one of clauses 407-422 wherein the concentration of the diafiltration buffer is between about 0.01 mM-10 mM, between about 0.1 mM-10 mM, between about 0.5 mM-10 mM, between about 1 mM-10 mM, between about 2 mM-10 mM, between about 3 mM-10 mM, between about 4 mM-10 mM, between about 5 mM-10 mM, between about 6 mM-10 mM, between about 7 mM-10 mM, between about 8 mM-10 mM or between about 9 mM-10 mM.
clause 428. The method of any one of clauses 407-422 wherein the concentration of the 35 diafiltration buffer is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
clause 429. The method of any one of clauses 407-422 wherein the concentration of the diafiltration buffer is about 0.1 mM, about 0.2 mM, about 1 mM, about 5 mM, about 10 mM, about 15 mM, about 20 mM, about 30 mM, about 40 mM, or about 50 mM.
clause 430. The method of any one of clauses 407-422 wherein the concentration of the diafiltration buffer is about 30 mM.
clause 431. The method of any one of clauses 407-422 wherein the concentration of the diafiltration buffer is about 25 mM.
clause 432. The method of any one of clauses 407-422 wherein the concentration of the diafiltration buffer is about 20 mM.
clause 433. The method of any one of clauses 407-422 wherein the concentration of the diafiltration buffer is about 15 mM.
clause 434. The method of any one of clauses 407-422 wherein the concentration of the diafiltration buffer is about 10 mM.
clause 435. The method of any one of clauses 401-434 wherein the replacement solution comprises a chelating agent.
clause 436. The method of any one of clauses 401-434 wherein the replacement solution comprises an alum chelating agent.
clause 437. The method of any one of clauses 401-434 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these.
clause 438. The method of any one of clauses 401-434 wherein the replacement solution comprises a chelating agent selected from the groups consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), a salt of citric acid (citrate) and combinations of these.
clause 439. The method of any one of clauses 401-434 wherein the replacement solution comprises Ethylene Diamine Tetra Acetate (EDTA) as chelating agent.
clause 440. The method of any one of clauses 401-434 wherein the replacement solution comprises a salt of citric acid (citrate) as chelating agent.
clause 441. The method of any one of clauses 401-434 wherein the replacement solution comprises sodium citrate as chelating agent.
clause 442. The method of any one of clauses 435-441 wherein the concentration of the chelating agent in the replacement solution is from 1 to 500 mM.
clause 443. The method of any one of clauses 435-441 wherein the concentration of the chelating agent in the replacement solution is from 2 to 400 mM.
clause 444. The method of any one of clauses 435-441 wherein concentration of the chelating agent in the replacement solution is from 10 to 400 mM.
clause 445. The method of any one of clauses 435-441 wherein concentration of the chelating agent in the replacement solution is from 10 to 200 mM.
clause 446. The method of any one of clauses 435-441 wherein concentration of the chelating agent in the replacement solution is from 10 to 100 mM.
clause 447. The method of any one of clauses 435-441 wherein concentration of the chelating agent in the replacement solution is from 10 to 50 mM.
clause 448. The method of any one of clauses 435-441 wherein concentration of the chelating agent in the replacement solution is from 10 to 30 mM.
clause 449. The method of any one of clauses 435-441 wherein concentration of the chelating agent in the replacement solution is about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 45 mM, about 50 mM, about 55 mM, about 60 mM, about 65 mM, about 70 mM, about 75 mM, about 80 mM, about 85 mM, about 90 mM, about 95 or about 100 mM.
clause 450. The method of any one of clauses 435-441 wherein concentration of the chelating agent in the replacement solution is about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.
clause 451. The method of any one of clauses 435-441 wherein concentration of the chelating agent in the replacement solution is about 15 mM, about 20 mM, about 25 mM, about 30 mM, about 35 mM, about 40 mM, about 45 mM or about 50 mM.
clause 452. The method of any one of clauses 407-451 wherein the replacement solution comprises a salt.
clause 453. The method of clause 452 wherein, the salt is selected from the groups consisting of magnesium chloride, potassium chloride, sodium chloride and a combination thereof.
clause 454. The method of clause 452 wherein, the salt is sodium chloride.
clause 455. The method of any one of clauses 432-434 wherein the replacement solution comprises sodium chloride at 1 about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250 or about 300 mM.
clause 456. The method of any one of clauses 401-455 wherein the number of diavolumes is at least 5, 10, 15, 20, 25, 30, 35, 40, 45, or 50.
clause 457. The method of any one of clauses 401-455 wherein the number of diavolumes is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95 or about 100.
clause 458. The method of any one of clauses 401-455 wherein the number of diavolumes is about 5, about 6, about 7, about 8, about 9, about 10, about 11, about 12, about 13, about 14 or about 15.
clause 459. The method of any one of clauses 401-458 wherein said dialfiltration step is performed at temperature of between about 20° C. to about 90° C.
clause 460. The method of any one of clauses 401-458 wherein said dialfiltration step is performed at temperature of between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 461. The method of any one of clauses 401-458 wherein said dialfiltration step is performed at temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 462. The method of any one of clauses 401-458 wherein said dialfiltration step is performed at temperature of about 50° C.
clause 463. The method of any one of clauses 379-458 wherein said ultrafiltration and dialfiltration steps if both conducted are performed at a temperature between about 20° C. to about 90° C.
clause 464. The method of any one of clauses 379-458 wherein said ultrafiltration and dialfiltration steps if both conducted are performed at a temperature between about 35° C. to about 80° C., at temperature between about 40° C. to about 70° C., at temperature between about 45° C. to about 65° C., at temperature between about 50° C. to about 60° C., at temperature between about 50° C. to about 55° C., at temperature between about 45° C. to about 55° C. or at temperature between about 45° C. to about 55° C.
clause 465. The method of any one of clauses 379-458 wherein said ultrafiltration and dialfiltration steps if both conducted are performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C.
clause 466. The method of any one of clauses 379-458 wherein said ultrafiltration and dialfiltration steps if both conducted are performed at a temperature of about 50° C.
clause 467. The method of any one of clauses 379-466 wherein said purified solution of polysaccharide is homogenized by sizing.
clause 468. The method of any one of clauses 379-466 wherein said purified solution of polysaccharide is subjected to mechanical sizing.
clause 469. The method of any one of clauses 379-466 wherein said purified solution of polysaccharide is subjected to High Pressure Homogenization Shearing.
clause 470. The method of any one of clauses 379-466 wherein said purified solution of polysaccharide is subjected to chemical hydrolysis.
clause 471. The method of any one of clauses 379-470 wherein said purified solution of polysaccharide is sized to a target molecular weight.
clause 472. The method of any one of clauses 379-471 wherein said purified solution of polysaccharide is sized to a molecular weight of between about 5 kDa and about 4,000 kDa.
clause 473. The method of any one of clauses 379-471 wherein said purified solution of polysaccharide is sized to a molecular weight of between about 10 kDa and about 4,000 kDa.
clause 474. The method of any one of clauses 379-471 wherein said purified solution of polysaccharide is sized to a molecular weight of between about 50 kDa and about 4,000 kDa.
clause 475. The method of any one of clauses 379-471 wherein said purified solution of polysaccharide is sized to a molecular weight of between about 50 kDa and about 3,500 kDa; between about 50 kDa and about 3,000 kDa; between about 50 kDa and about 2,500 kDa; between about 50 kDa and about 2,000 kDa; between about 50 kDa and about 1,750 kDa; about between about 50 kDa and about 1,500 kDa; between about 50 kDa and about 1,250 kDa; between about 50 kDa and about 1,000 kDa; between about 50 kDa and about 750 kDa; between about 50 kDa and about 500 kDa; between about 100 kDa and about 4,000 kDa; between about 100 kDa and about 3,500 kDa; about 100 kDa and about 3,000 kDa; about 100 kDa and about 2,500 kDa; about 100 kDa and about 2,250 kDa; between about 100 kDa and about 2,000 kDa; between about 100 kDa and about 1,750 kDa; between about 100 kDa and about 1,500 kDa; between about 100 kDa and about 1,250 kDa; between about 100 kDa and about 1,000 kDa; between about 100 kDa and about 750 kDa; between about 100 kDa and about 500 kDa; between about 200 kDa and about 4,000 kDa; between about 200 kDa and about 3,500 kDa; between about 200 kDa and about 3,000 kDa; between about 200 kDa and about 2,500 kDa; between about 200 kDa and about 2,250 kDa; between about 200 kDa and about 2,000 kDa; between about 200 kDa and about 1,750 kDa; between about 200 kDa and about 1,500 kDa; between about 200 kDa and about 1,250 kDa; between about 200 kDa and about 1,000 kDa; between about 200 kDa and about 750 kDa; or between about 200 kDa and about 500 kDa. In further such embodiments, the polysaccharide the purified polysaccharide is sized to a molecular weight of between about 250 kDa and about 3,500 kDa; between about 250 kDa and about 3,000 kDa; between about 250 kDa and about 2,500 kDa; between about 250 kDa and about 2,000 kDa; between about 250 kDa and about 1,750 kDa; about between about 250 kDa and about 1,500 kDa; between about 250 kDa and about 1,250 kDa; between about 250 kDa and about 1,000 kDa; between about 250 kDa and about 750 kDa; between about 250 kDa and about 500 kDa; between about 300 kDa and about 4,000 kDa; between about 300 kDa and about 3,500 kDa; about 300 kDa and about 3,000 kDa; about 300 kDa and about 2,500 kDa; about 300 kDa and about 2,250 kDa; between about 300 kDa and about 2,000 kDa; between about 300 kDa and about 1,750 kDa; between about 300 kDa and about 1,500 kDa; between about 300 kDa and about 1,250 kDa; between about 300 kDa and about 1,000 kDa; between about 300 kDa and about 750 kDa; between about 300 kDa and about 500 kDa; between about 500 kDa and about 4,000 kDa; between about 500 kDa and about 3,500 kDa; between about 500 kDa and about 3,000 kDa; between about 500 kDa and about 2,500 kDa; between about 500 kDa and about 2,250 kDa; between about 500 kDa and about 2,000 kDa; between about 500 kDa and about 1,750 kDa; between about 500 kDa and about 1,500 kDa; between about 500 kDa and about 1,250 kDa; between about 500 kDa and about 1,000 kDa; between about 500 kDa and about 750 kDa; or between about 500 kDa and about 600 kDa.
clause 476. The method of any one of clauses 379-471 wherein said purified solution of polysaccharide is sized to a molecular weight of about 5 kDa, about 10 kDa, about 15 kDa, about 20 kDa, about 25 kDa, about 30 kDa, about 35 kDa, about 40 kDa, about 45 kDa, about 50 kDa, about 75 kDa, about 90 kDa, about 100 kDa, about 150 kDa, about 200 kDa, about 250 kDa, about 300 kDa, about 350 kDa, about 400 kDa, about 450 kDa, about 500 kDa, about 550 kDa, about 600 kDa, about 650 kDa, about 700 kDa, about 750 kDa, about 800 kDa, about 850 kDa, about 900 kDa, about 950 kDa, about 1000 kDa, about 1250 kDa, about 1500 kDa, about 1750 kDa, about 2000 kDa, about 2250 kDa, about 2500 kDa, about 2750 kDa, about 3000 kDa, about 3250 kDa, about 3500 kDa, about 3750 kDa or about 4,000 kDa.
clause 477. The method of any one of clauses 1-746 wherein said purified solution of polysaccharide is sterilely filtered.
clause 478. The method of clause 477 wherein said sterile filtration is dead-end filtration.
clause 479. The method of clause 477 wherein said sterile filtration is tangential filtration.
clause 480. The method of any one of clauses 477-479 wherein the filter has a nominal retention range of between about 0.01-0.2 micron, about 0.05-0.2 micron, about 0.1-0.2 micron or about 0.15-0.2 micron.
clause 481. The method of any one of clauses 477-479 wherein the filter has a nominal retention range of about 0.05, about 0.1, about 0.15 or about 0.2 micron.
clause 482. The method of any one of clauses 477-479 wherein the filter has a nominal retention range of about 0.2 micron.
clause 483. The method of any one of clauses 477-482 wherein the filter has a filter capacity of about 25-1500 L/m², 50-1500 L/m², 75-1500 L/m², 100-1500 L/m², 150-1500 L/m², 200-1500 L/m², 250-1500 L/m², 300-1500 L/m², 350-1500 L/m², 400-1500 L/m², 500-1500 L/m², 750-1500 L/m², 1000-1500 L/m² or 1250-1500 L/m².
clause 484. The method of any one of clauses 477-482 wherein the filter has a filter capacity of about 25-1000 L/m², 50-1000 L/m², 75-1000 L/m², 100-1000 L/m², 150-1000 L/m², 200-1000 L/m², 250-1000 L/m², 300-1000 L/m², 350-1000 L/m², 400-1000 L/m², 500-1000 L/m² or 750-1000 L/m².
clause 485. The method of any one of clauses 477-482 wherein the filter has a filter capacity of a filter capacity of 25-500 L/m², 50-500 L/m², 75-500 L/m², 100-500 L/m², 150-500 L/m², 200-500 L/m², 250-500 L/m², 300-500 L/m², 350-500 L/m² or 400-500 L/m².
clause 486. The method of any one of clauses 477-482 wherein the filter has a filter capacity of 25-300 L/m², 50-300 L/m², 75-300 L/m², 100-300 L/m², 150-300 L/m², 200-300 L/m² or 250-300 L/m².
clause 487. The method of any one of clauses 477-482 wherein the filter has a filter capacity of 25-250 L/m², 50-250 L/m², 75-250 L/m², 100-250 L/m² or 150-250 L/m², 200-250 L/m².
clause 488. The method of any one of clauses 477-482 wherein the filter has a filter capacity of 25-100 L/m², 50-100 L/m² or 75-100 L/m².
clause 489. The method of any one of clauses 477-482 wherein the filter has a filter capacity of about 25, about 50, about 75, about 100, about 150, about 200, about 250, about 300, about 350, about 400, about 500, about 600, about 700, about 800, about 900, about 1000, about 1100, about 1200, about 1300, about 1400 or about 1500 L/m².
clause 490. The method of any one of clauses 1-489 wherein the obtained purified polysaccharide is in liquid solution.
clause 491. The method of any one of clauses 1-489 wherein the obtained purified polysaccharide is a dried powder.
clause 492. The method of any one of clauses 1-489 wherein the obtained purified polysaccharide solution is lyophilized.
clause 493. The method of any one of clauses 1-489 or 492 wherein the obtained purified polysaccharide solution is a freeze-dried cake.
clause 494. The method of any one of clauses 1-493 wherein said bacterial polysaccharide is a capsular polysaccharide, a sub-capsular polysaccharide, or a lipopolysaccharide.
clause 495. The method of any one of clauses 1-493 wherein said bacterial polysaccharide is a capsular polysaccharide.
clause 496. The method of any one of clauses 1-493 wherein said bacterial polysaccharide is a capsular polysaccharide from Staphylococcus aureus.
clause 497. The method of any one of clauses 1-493 wherein said bacterial polysaccharide is the capsular polysaccharide from Staphylococcus aureus type 5.
clause 498. The method of any one of clauses 1-493 wherein said bacterial polysaccharide is the capsular polysaccharide from Staphylococcus aureus type 8.
clause 499. The method of any one of clauses 1-493 wherein said bacterial polysaccharide is a capsular polysaccharide from Enterococcus faecalis.
clause 500. The method of any one of clauses 1-493 wherein said bacterial polysaccharide is the capsular polysaccharide from Haemophilus influenzae type b.
clause 501. The method of any one of clauses 1-493 wherein said bacterial polysaccharide is a capsular polysaccharide from Neisseria meningitidis.
clause 502. The method of any one of clauses 1-493 wherein said bacterial polysaccharide is the capsular polysaccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC).
clause 503. The method of any one of clauses 1-493 wherein said bacterial polysaccharide is the capsular polysaccharide from N. meningitidis serogroup A (MenA).
clause 504. any one of clauses 1-493 wherein said bacterial polysaccharide is the capsular polysaccharide from N. meningitidis serogroup W135 (MenW135).
clause 505. The method of any one of clauses 1-493 wherein said bacterial polysaccharide is the capsular polysaccharide from N. meningitidis serogroup A (MenY).
clause 506. The method of any one of clauses 1-493 wherein said bacterial polysaccharide is the capsular polysaccharide from N. meningitidis serogroup A (MenX).
clause 507. The method of any one of clauses 1-493 wherein said bacterial polysaccharide is the capsular polysaccharide from N. meningitidis serogroup A (MenC).
clause 508. The method of any one of clauses 1-493 wherein said bacterial polysaccharide is a capsular polysaccharide from Escherichia coli.
clause 509. The method of any one of clauses 1-493 wherein said bacterial polysaccharide is a capsular polysaccharide from Streptococcus agalactiae (Group B streptococcus (GBS)).
clause 510. The method of any one of clauses 1-493 wherein said bacterial polysaccharide is a capsular polysaccharide selected from the group consisting of the capsular polysaccharide from GBS types Ia, Ib, II, III, IV, V, VI, VII and VIII.
clause 511. The method of any one of clauses 1-493 wherein said bacterial polysaccharide is a capsular polysaccharide from an Escherichia coli strain part of the Enterovirulent Escherichia coli group (EEC Group).
clause 512. The method of any one of clauses 1-493 wherein said bacterial polysaccharide is a capsular polysaccharide from an Escherichia coli strain part of the Enterovirulent Escherichia coli group (EEC Group) such as Escherichia coli—enterotoxigenic (ETEC), Escherichia coli-enteropathogenic (EPEC), Escherichia coli—O157:H7 enterohemorrhagic (EHEC), or Escherichia coli—enteroinvasive (EIEC). In an embodiment, the source of bacterial capsular polysaccharide is an Uropathogenic Escherichia coli (UPEC).
clause 513. The method of any one of clauses 1-493 wherein said bacterial polysaccharide is a capsular polysaccharide from an Escherichia coli serotype selected from the group consisting of serotypes O157:H7, O26:H11, O111:H- and O103:H2.
clause 514. The method of any one of clauses 1-493 wherein said bacterial polysaccharide is a capsular polysaccharide from an Escherichia coli serotype selected from the group consisting of serotypes O6:K2:H1 and O18:K1:H7.
clause 515. The method of any one of clauses 1-493 wherein said bacterial polysaccharide is a capsular polysaccharide from an Escherichia coli serotype selected from the group consisting of serotypes O45:K1, O17:K52:H18, O19:H34 and O7:K1.
clause 516. The method of any one of clauses 1-493 wherein said bacterial polysaccharide is a capsular polysaccharide from an Escherichia coli serotype O104:H4.
clause 517. The method of any one of clauses 1-493 wherein said bacterial polysaccharide is a capsular polysaccharide from an Escherichia coli serotype O1:K12:H7.
clause 518. The method of any one of clauses 1-493 wherein said bacterial polysaccharide is a capsular polysaccharide from an Escherichia coli serotype O127:H6.
clause 519. The method of any one of clauses 1-493 wherein said bacterial polysaccharide is a capsular polysaccharide from an Escherichia coli serotype O139:H28.
clause 520. The method of any one of clauses 1-493 wherein said bacterial polysaccharide is a capsular polysaccharide from an Escherichia coli serotype O128:H2.
clause 521. The method of any one of clauses 1-493 wherein said bacterial polysaccharide is a capsular polysaccharide from Streptococcus pneumoniae.
clause 522. The method of any one of clauses 1-493 wherein said bacterial polysaccharide is the capsular polysaccharide from a Streptococcus pneumoniae serotype selected from the group consisting of serotypes 1, 2, 3, 4, 5, 6A, 6B, 6C, 7F, 8, 9V, 9N, 10A, 11A, 12F, 14, 15A, 15B, 15C, 16F, 17F, 18C, 19A, 19F, 20, 22F, 23A, 23B, 23F, 24B, 24F, 29, 31, 33F, 34, 35B, 35F, 38, 72 and 73.
clause 523. The method of any one of clauses 1-493 wherein said bacterial polysaccharide is the capsular polysaccharide from a Streptococcus pneumoniae serotype selected from the group consisting of serotypes 1, 2, 3, 4, 5, 6A, 6B, 7F, 8, 9V, 9N, 10A, 11A, 12F, 14, 15A, 15B, 15C, 16F, 17F, 18C, 19A, 19F, 20, 22F, 23A, 23B, 23F, 24F, 29, 31, 33F, 35B, 35F, 38, 72 and 73.
clause 524. The method of any one of clauses 1-493 wherein said bacterial polysaccharide is the capsular polysaccharide from a Streptococcus pneumoniae serotype selected from the group consisting of serotypes 8, 10A, 11A, 12F, 15B, 22F and 33F.
clause 525. The method of any one of clauses 1-493 wherein said bacterial polysaccharide is the capsular polysaccharide from Streptococcus pneumoniae serotype 1.
clause 526. The method of any one of clauses 1-493 wherein said bacterial polysaccharide is the capsular polysaccharide from Streptococcus pneumoniae serotype 2.
clause 527. The method of any one of clauses 1-493 wherein said bacterial polysaccharide is the capsular polysaccharide from Streptococcus pneumoniae serotype 3.
clause 528. The method of any one of clauses 1-493 wherein said bacterial polysaccharide is the capsular polysaccharide from Streptococcus pneumoniae serotype 4.
clause 529. The method of any one of clauses 1-493 wherein said bacterial polysaccharide is the capsular polysaccharide from Streptococcus pneumoniae serotype 5.
clause 530. The method of any one of clauses 1-493 wherein said bacterial polysaccharide is the capsular polysaccharide from Streptococcus pneumoniae serotype 6A.
clause 531. The method of any one of clauses 1-493 wherein said bacterial polysaccharide is the capsular polysaccharide from Streptococcus pneumoniae serotype 6B.
clause 532. The method of any one of clauses 1-493 wherein said bacterial polysaccharide is the capsular polysaccharide from Streptococcus pneumoniae serotype 6C.
clause 533. The method of any one of clauses 1-493 wherein said bacterial polysaccharide is the capsular polysaccharide from Streptococcus pneumoniae serotype 7F.
clause 534. The method of any one of clauses 1-493 wherein said bacterial polysaccharide is the capsular polysaccharide from Streptococcus pneumoniae serotype 8.
clause 535. The method of any one of clauses 1-493 wherein said bacterial polysaccharide is the capsular polysaccharide from Streptococcus pneumoniae serotype 9V.
clause 536. The method of any one of clauses 1-493 wherein said bacterial polysaccharide is the capsular polysaccharide from Streptococcus pneumoniae serotype 9N.
clause 537. The method of any one of clauses 1-493 wherein said bacterial polysaccharide is the capsular polysaccharide from Streptococcus pneumoniae serotype 10A.
clause 538. The method of any one of clauses 1-493 wherein said bacterial polysaccharide is the capsular polysaccharide from Streptococcus pneumoniae serotype 11A.
clause 539. The method of any one of clauses 1-493 wherein said bacterial polysaccharide is the capsular polysaccharide from Streptococcus pneumoniae serotype 12F.
clause 540. The method of any one of clauses 1-493 wherein said bacterial polysaccharide is the capsular polysaccharide from Streptococcus pneumoniae serotype 14.
clause 541. The method of any one of clauses 1-493 wherein said bacterial polysaccharide is the capsular polysaccharide from Streptococcus pneumoniae serotype 15A.
clause 542. The method of any one of clauses 1-493 wherein said bacterial polysaccharide is the capsular polysaccharide from Streptococcus pneumoniae serotype 15B.
clause 543. The method of any one of clauses 1-493 wherein said bacterial polysaccharide is the capsular polysaccharide from Streptococcus pneumoniae serotype 15C.
clause 544. The method of any one of clauses 1-493 wherein said bacterial polysaccharide is the capsular polysaccharide from Streptococcus pneumoniae serotype 16F.
clause 545. The method of any one of clauses 1-493 wherein said bacterial polysaccharide is the capsular polysaccharide from Streptococcus pneumoniae serotype 17F.
clause 546. The method of any one of clauses 1-493 wherein said bacterial polysaccharide is the capsular polysaccharide from Streptococcus pneumoniae serotype 18C.
clause 547. The method of any one of clauses 1-493 wherein said bacterial polysaccharide is the capsular polysaccharide from Streptococcus pneumoniae serotype 19A.
clause 548. The method of any one of clauses 1-493 wherein said bacterial polysaccharide is the capsular polysaccharide from Streptococcus pneumoniae serotype 19F.
clause 549. The method of any one of clauses 1-493 wherein said bacterial polysaccharide is the capsular polysaccharide from Streptococcus pneumoniae serotype 20.
clause 550. The method of any one of clauses 1-493 wherein said bacterial polysaccharide is the capsular polysaccharide from Streptococcus pneumoniae serotype 20A.
clause 551. The method of any one of clauses 1-493 wherein said bacterial polysaccharide is the capsular polysaccharide from Streptococcus pneumoniae serotype 20B.
clause 552. The method of any one of clauses 1-493 wherein said bacterial polysaccharide is the capsular polysaccharide from Streptococcus pneumoniae serotype 22F.
clause 553. The method of any one of clauses 1-493 wherein said bacterial polysaccharide is the capsular polysaccharide from Streptococcus pneumoniae serotype 23A.
clause 554. The method of any one of clauses 1-493 wherein said bacterial polysaccharide is the capsular polysaccharide from Streptococcus pneumoniae serotype 23B.
clause 555. The method of any one of clauses 1-493 wherein said bacterial polysaccharide is the capsular polysaccharide from Streptococcus pneumoniae serotype 23F.
clause 556. The method of any one of clauses 1-493 wherein said bacterial polysaccharide is the capsular polysaccharide from Streptococcus pneumoniae serotype 24B.
clause 557. The method of any one of clauses 1-493 wherein said bacterial polysaccharide is the capsular polysaccharide from Streptococcus pneumoniae serotype 24F.
clause 558. The method of any one of clauses 1-493 wherein said bacterial polysaccharide is the capsular polysaccharide from Streptococcus pneumoniae serotype 29.
clause 559. The method of any one of clauses 1-493 wherein said bacterial polysaccharide is the capsular polysaccharide from Streptococcus pneumoniae serotype 31.
clause 560. The method of any one of clauses 1-493 wherein said bacterial polysaccharide is the capsular polysaccharide from Streptococcus pneumoniae serotype 33F.
clause 561. The method of any one of clauses 1-493 wherein said bacterial polysaccharide is the capsular polysaccharide from Streptococcus pneumoniae serotype 34.
clause 562. The method of any one of clauses 1-493 wherein said bacterial polysaccharide is the capsular polysaccharide from Streptococcus pneumoniae serotype 35B.
clause 563. The method of any one of clauses 1-493 wherein said bacterial polysaccharide is the capsular polysaccharide from Streptococcus pneumoniae serotype 35F.
clause 564. The method of any one of clauses 1-493 wherein said bacterial polysaccharide is the capsular polysaccharide from Streptococcus pneumoniae serotype 38.
clause 565. The method of any one of clauses 1-493 wherein said bacterial polysaccharide is the capsular polysaccharide from Streptococcus pneumoniae serotype 72.
clause 566. The method of any one of clauses 1-493 wherein said bacterial polysaccharide is the capsular polysaccharide from Streptococcus pneumoniae serotype 73.
clause 567. A purified bacterial polysaccharide obtained by the method of any one of clauses 1-566.
clause 568. A purified bacterial polysaccharide obtainable by the method of any one of clauses 1-566.
clause 569. A purified bacterial polysaccharide obtained by the method of any one of clauses 1-566 for use as an antigen.
clause 570. A purified bacterial polysaccharide obtained by the method of any one of clauses 1-566 conjugated to carrier protein.
clause 571. A purified bacterial polysaccharide obtained by the method of any one of clauses 1-566 further conjugated to a carrier protein.
clause 572. A glycoconjugate of a purified bacterial polysaccharide obtained by the method of any one of clauses 1-566.
clause 573. An immunogenic composition comprising any of the purified polysaccharide of any one of clauses 567-568.
clause 574. An immunogenic composition comprising a glycoconjugate of any one of clauses 571-572.
clause 575. An immunogenic composition comprising any of the glycoconjugate disclosed herein.
clause 576. An immunogenic composition comprising any of the combination of glycoconjugates disclosed herein.
clause 577. A method for purifying a capsular polysaccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC) from a solution comprising said polysaccharide together with contaminants, wherein said method comprises a flocculation step and a chromatography step.
clause 578. A method of clause 577 wherein the chromatography step is a Hydrophobic Interaction Chromatography (HIC) step.
clause 579. A method of clause 578 wherein said HIC step is as defined in clauses 359 to 378.
clause 580. A method of clause 578 and of any of clauses 2 to 493.
clause 581. A method of any of clauses 578 to 5809 wherein said bacterial polysaccharide is the capsular polysaccharide from N. meningitidis serogroup A (MenC).
clause 582. A method according to clause 581 wherein the method further comprise an ionic exchange chromatography step before the HIC step.
clause 583. A method according to clause 582 wherein the method does not comprise an activated carbon filtration step.

What is claimed is:

1. A method for purifying a saccharide derived from bacteria from a solution comprising said saccharide and contaminants following fermentation, wherein said method comprises the following steps:
a) acid hydrolysis;
b) a first ultrafiltration/diafiltration-(UFDF-1);
b) carbon filtration;
c) chromatography; and
d) a second ultrafiltration/diafiltration-(UFDF-2).

2. The method of claim 1, further comprising a flocculation step following the acid hydrolysis of step (a).

3. The method of claim 1 or 2, wherein the chromatography of step (c) comprises IEX membrane chromatography or Hydrophobic Interaction Chromatography (HIC) or both.

4. The method of any one of claims 1-3, wherein the bacteria is a gram positive bacteria.

5. The method of claim 4, wherein the bacteria is any one of Streptococcus, Staphylococcus, Enterococci, Bacillus, Corynebacterium, Listeria, Erysipelothrix, or Clostridium.

6. The method of claim 5, wherein the bacteria is any one of Streptococcus pneumoniae, Streptococcus pyogenes, Streptococcus agalactiae, Group C & G Streptococcii or Staphylococcus aureus.

7. The method of any one of claims 1-3, wherein the bacteria is a gram negative bacteria.

8. The method of claim 7, wherein the bacteria is any one of Haemophilus, Neisseria, Escherichia or Klebsiella.

9. The method of claim 8, wherein the bacteria is Haemophilus influenzae, Neisseria meningitidis, Escherichia coli or Klebsiella pneumoniae.

10. The method of claim 9, wherein the bacteria is Escherichia coli comprising a saccharide having a structure selected from any one of Formula O1, Formula O1A, Formula O1B, Formula O1C, Formula O2, Formula O3, Formula O4, Formula O4:K52, Formula O4:K6, Formula O5, Formula O5ab, Formula O5ac, Formula O6, Formula O6:K2; K13; K15, Formula O6:K54, Formula O7, Formula O8, Formula O9, Formula O10, Formula O11, Formula O12, Formula O13, Formula O14, Formula O15, Formula O16, Formula O17, Formula O18, Formula O18A, Formula O18ac, Formula O18A1, Formula O18B, Formula O18B1, Formula O19, Formula O20, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O34, Formula O35, Formula O36, Formula O37, Formula O38, Formula O39, Formula O40, Formula O41, Formula O42, Formula O43, Formula O44, Formula O45, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O89, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O144, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O175, Formula O176, Formula O177, Formula O178, Formula O179, Formula O180, Formula O181, Formula O182, Formula O183, Formula O184, Formula O185, Formula O186 or Formula O187.

11. The method of claim 9, wherein the bacteria is Klebsiella pneumoniae comprising a saccharide having a structure selected from any one of Formula K.O1.1, Formula K.O1.2, Formula K.O1.3, Formula K.O1.4, Formula K.O2.1, Formula K.O2.2, Formula K.O2.3, Formula K.O2.4, Formula K.O3, Formula K.O4, Formula K.O5, Formula K.O7, Formula K.O12 or Formula K.O8.


SEQUENCE LISTING (24 sequences)

SEQ ID NO: 1 - DNA, 22 bp, Artificial Sequence
  Synthetic sequence; LT2wzzB_S
gaagcaaaccgtacgcgtaaag

SEQ ID NO: 2 - DNA, 20 bp, Artificial Sequence
  Synthetic sequence; LT2wzzB_AS
cgaccagctcttacacggcg

SEQ ID NO: 3 - DNA, 36 bp, Artificial Sequence
  Synthetic sequence; O25bFepE_S
gaaataggaccactaataaatacacaaattaataac

SEQ ID NO: 4 - DNA, 21 bp, Artificial Sequence
  Synthetic sequence; O25bFepE_A
ataattgacgatccggttgcc

SEQ ID NO: 5 - DNA, 27 bp, Artificial Sequence
  Synthetic sequence; wzzB P1_S
gctatttacgccctgattgtcttttgt

SEQ ID NO: 6 - DNA, 22 bp, Artificial Sequence
  Synthetic sequence; wzzB P2_AS
attgagaacctgcgtaaacggc

SEQ ID NO: 7 - DNA, 24 bp, Artificial Sequence
  Synthetic sequence; wzzB P3_S
tgaagagcggttcagataacttcc

SEQ ID NO: 8 - DNA, 21 bp, Artificial Sequence
  Synthetic sequence; wzzB P4_AS
cgatccggaaacctcctacac

SEQ ID NO: 9 - DNA, 26 bp, Artificial Sequence
  Synthetic sequence; O157 FepE_S
gattattcgcgcaacgctaaacagat

SEQ ID NO: 10 - DNA, 23 bp, Artificial Sequence
  Synthetic sequence; O157 FepE_AS
tgatcattgacgatccggtagcc

SEQ ID NO: 11 - DNA, 70 bp, Artificial Sequence
  Synthetic sequence; pBAD33_adaptor_S
cggtagctgtaaagccaggggcggtagcgtggtttaaacccaagcaacagatcggcgtcgtcggtatgga

SEQ ID NO: 12 - DNA, 78 bp, Artificial Sequence
  Synthetic sequence; pBAD33_adaptor_AS
agcttccataccgacgacgccgatctgttgcttgggtttaaaccacgctaccgcccctggctttacagctaccgagct

SEQ ID NO: 13 - DNA, 30 bp, Artificial Sequence
  Synthetic sequence; JUMPSTART_r
ggtagctgtaaagccaggggcggtagcgtg

SEQ ID NO: 14 - DNA, 30 bp, Artificial Sequence
  Synthetic sequence; gnd_f
ccataccgacgacgccgatctgttgcttgg

SEQ ID NO: 15 - protein, 377 aa, Escherichia coli
MSSLNIKQGSDAHFPDYPLASPSNNEIDLLNLISVLWRAKKTVMAVVFAFACAGLLISFILPQKWTSAAVVTPPEPVQWQELEKSFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISTLVVKESLENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKAHVNDVNFTPFKYQLSPSLPVKKDGPGKAIIVILSALIGGMVACGGVLLRYAMASRKQDAMMADHLV

SEQ ID NO: 16 - protein, 377 aa, Escherichia coli
MSSLNIKQGSEAHFPEYPLASPSNNEIDLLNLIEVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDPLDLHRAIVALSEKMKAVDDNASKKKDESALYTSWTLSFTAPTSEEAQKVLAGYIDYISALVVKESIENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKTNINDVNFTPFKYQLRPSLPVKKDGQGKAIIVILSALVGGMVACGGVLLRHAMASRKQDAMMADHLV

SEQ ID NO: 17 - protein, 377 aa, Escherichia coli; identical to SEQ ID NO: 15

SEQ ID NO: 18 - protein, 377 aa, Escherichia coli
MSSLNIKQGSDAHFPDYPLASPSNNEIDLLNLISVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISALVVKESIENVRNKLEIKTQFEKEKLAQDRIKMKNQLDANIQRLNYSLDIANAAGIKKPVYSNGQAVKDDPDFSISLGADGIERKLEIEKAVTDVAELNGELRNRQYLVEQLTKANINDVNFTPFKYQLSPSLPVKKDGPGKAIIVILSALIGGMVACGSVLLRYAMASRKQDAMMADHLV

SEQ ID NO: 19 - protein, 378 aa, Salmonella enterica
MPSLNVKQEKNQSFAGYSLPPANSHEIDLFSLIEVLWQAKRRILATVFAFACVGLLLSFLLPQKWTSQAIVTPAESVQWQGLERTLTALRVLDMEVSVDRGSVFNLFIKKFSSPSLLEEYLRSSPYVMDQLKGAQIDEQDLHRAIVLLSEKMKAVDSNVGKKNETSLFTSWTLSFTAPTREEAQKVLAGYIQYISDIVVKETLENIRNQLEIKTRYEQEKLAMDRVRLKNQLDANIQRLHYSLEIANAAGIKRPVYSNGQAVKDDPDFSISLGADGISRKLEIEKGVTDVAEIDGDLRNRQYHVEQLAAMNVSDVKFTPFKYQLSPSLPVKKDGPGKAIIIILAALIGGMMACGGVLLRHAMVSRKMENALAIDERLV

SEQ ID NO: 20 - protein, 325 aa, Escherichia coli
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQEQVTKPQVQQTEDVTQDTLFLLGSEALESMIKHEATRPLVFSSNYYQTRQNLLDIESLKVDDLDIHAYRYVMKPTLPIRRDSPKKAITLILAVLLGGMVGAGIVLGRNALRNYNAK

SEQ ID NO: 21 - protein, 326 aa, Escherichia coli
MRVENNNVSGQNNDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQDEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPTLPIRRDSPKKAITLILAVLLGGMVGAGIVLGRNALRNYNAK

SEQ ID NO: 22 - protein, 326 aa, Escherichia coli
MRVENNNVSGQNHDPEQIDLIDLLVQLWRGKMTIIISVVVAIALAIGYLAVAKEKWTSTAIITQPDVGQIAGYNNAMNVIYGQAAPKVSDLQETLIGRFSFAFSALAETLDNQKEPEKLTIEPSVKNQQLPLTVSYVGQTAEDAQMKLAQYIQQVDDKVNQELEKDLKDNLALGRKNLQDSLRTQEVVAQEQKDLRIRQIQEALQYANQAQVTKPQIQQTGEDITQDTLFLLGSEALESMIKHEATRPLVFSPNYYQTRQNLLDIENLKVDDLDIHAYRYVMKPTLPIRRDSPKKAITLILAVLLGGMVGAGIVLGRNALRNYNSK

SEQ ID NO: 23 - protein, 326 aa, Escherichia coli
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEREKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPMLPIRRDSPKKAITLILAVLLGGMVGAGIVLGRNALRNYNAK

SEQ ID NO: 24 - protein, 327 aa, Salmonella enterica
MTVDSNTSSGRGNDPEQIDLIELLLQLWRGKMTIIVAVIIAILLAVGYLMIAKEKWTSTAIITQPDAAQVATYTNALNVLYGGNAPKISEVQANFISRFSSAFSALSEVLDNQKEREKLTIEQSVKGQALPLSVSYVSTTAEGAQRRLAEYIQQVDEEVAKELEVDLKDNITLQTKTLQESLETQEVVAQEQKDLRIKQIEEALRYADEAKITQPQIQQTQDVTQDTMFLLGSDALKSMIQNEATRPLVFSPAYYQTKQTLLDIKNLKVTADTVHVYRYVMKPTLPVRRDSPKTAITLVLAVLLGGMIGAGIVLGRNALRSYKPKAL